(19) United States
(12) Patent Application Publication
(10) Pub. No.: US 2011/0045997 A1
(43) Pub. Date: Feb. 24, 2011

(54) METHOD AND PRODUCT FOR "IN VITRO" GENOTYPING WITH APPLICATIONS IN ANTI-AGEING MEDICINE

(75) Inventors: Diego Tejedor Hernández, Derio - Vizcaya (ES); Laureano Simón Buela, Derio-Vizcaya (ES); Antonio Martínez Martínez, Derio-Vizcaya (ES); José Ingacio Lao Villadóniga, Derio-Vizcaya (ES)

Correspondence Address: MOORE & VAN ALLEN PLLC, P.O. BOX 13706, Research Triangle Park, NC, 27709, US

(73) Assignees: PROGENIKA BIOPHARMA, S.A., Derio - Vizcaya (ES); SABIOBBI, S.L., Madrid (ES)

(21) Appl. No.: 12/528,159

(22) PCT Filed: Feb. 21, 2008
(86) PCT No.: PCT/ES2008/000094

§ 371 Date: Nov. 11, 2010

(30) Foreign Application Priority Data
Feb. 23, 2007 (ES) P200700493

(51) Int. Cl. C40B 30/04; C40B 40/06; C07H 21/04; G06F 17/10

(52) U.S. Cl. 506/9; 506/16; 536/24.33; 703/2

(57) ABSTRACT

The invention relates to an “in vitro” method for determining the global genetic risk a subject has of developing a pathology associated with aging. Said method is based on the combination of particular genetic risks of developing common pathologies associated with aging. Said particular genetic risks are determined from the results obtained from the simultaneous genotyping of certain genetic variations associated with said pathologies associated with aging and the main objective of which is the use thereof in anti-aging medicine.

### FIELD OF THE INVENTION

The invention is comprised in the technical-industrial sector of the extracorporeal in vitro diagnosis of biological samples for the detection of gene variants, for example, polymorphisms or genetic mutations, associated with diseases associated with aging, or associated with the response to pharmacological treatments, with application in anti-aging medicine; the invention particularly relates to an in vitro method for determining the global genetic risk a subject has of developing a pathology associated with aging from a combination of particular genetic risks. The invention also relates to reagents and kits for putting said method into practice.

### BACKGROUND OF THE INVENTION

As a result of the knowledge obtained from the analysis of the human genome, many examples of alleles defined by single nucleotide polymorphisms or SNPs which can affect the good functioning of a certain system and others which, on the contrary, have a beneficial effect are currently known. It is important to bear in mind that many of these genes interact with one another and, for this reason, some antagonistic effects usually mutually compensate their expression, which can clinically be translated into the suppression of a certain sign or symptom within the clinical symptomotology. Nevertheless, in other cases the effects of some genes are mutually enhanced and as a result of this synergy, there may be both clinical and therapeutic response complications or peculiarities which explain the differences observed in the evolution of several cases with one and the same disease.

These differences are also shown in the predisposition to suffer from various common diseases and to the development of their complications. For example, the genetic susceptibility to dyslipidemias will most likely lead to a shorter life, on the other hand, inheriting gene variants in genes protecting against coronary diseases, against oxidative damage or against cancer will without a doubt aid to prolonging life. In this sense, there is a balance which can be established between genes with negative or deleterious effects (predisposing to diseases) and genes with positive effects (certain protective genotypes) in the maintenance of life reserves. Of course, it must never be forgotten that other non-genetic risk factors with a negative effect (unhealthy lifestyles and habits) in contrast to those with a positive effect (control of said habits, specific pharmacological intervention) which shift the balance in one direction or the other, play an important direct role in this genetic interaction, completing the modulation of the final clinical phenotype and finally determining the greater or lower life expectancy.

Medical treatments also have an effect among these environmental factors capable of modulating the expression of the genes. If they are the suitable ones, they would contribute to increasing survival once any disease has developed.

A genetic analysis can facilitate the very early detection of the particular vulnerability of each individual analyzed and at the same time it offers the possibility of providing a scientific basis to a treatment, which stops being empirical and general to become completely objective, since it will be formulated according to the principles of pharmacogenetics: a state-of-the-art tool which is gradually becoming the latest great revolution of modern medicine: the era of the personalized medicine.

If, furthermore, there is the possibility of analyzing the genetic polymorphisms involved in the etiopathogenesis of the disease, a comprehensive analysis of the problem could be conducted, under a unitary perspective including, on one hand, classic risk factors and on the other hand, the data obtained from the gene variants studied.

Until the mid twentieth century, it has been assumed that the diseases to which elderly people were more vulnerable, such as for example osteoporosis, were inevitable attributes of the aging process. It is true that aging predisposes to increasing the vulnerability to the disease, however, a large amount of research aimed at obtaining information about the biology of aging and longevity is currently being conducted.

Anti-aging medicine can be defined as any intervention delaying the development of pathologies related to aging and other adverse changes related to age and which are officially not listed as such diseases.

A number of molecular markers in the genome which are related to pathologies associated with aging have been described in recent years. Given that the list of genetic risk factors for developing a pathology associated with aging is increasingly numerous and the interest for considering its importance in the determinism of the disease continues to increase, it is currently necessary to have tools which allow quickly conducting the analysis of all these genetic factors as a whole.

The most relevant diseases associated with aging are those which occupy the first places among the main causes of morbimortality among people above 65 years of age, including cardiovascular diseases, cancer and osteoporosis. Aging has also been defined as the process resulting from an imperfect protection of the main cell components against oxidative stress. Furthermore, as people get older, drugs remain more time in the organism due to the decrease of the amount of water, therefore the prescription of suitable doses according to the response of the patient to the drugs becomes more important in order to prevent adverse reactions to such drugs.

### Vascular Disease

Vascular disease (VD) is one of the main causes of mortality and morbidity, therefore the development of models for predicting the risk of suffering from this type of disease, both for attempting to know the possible mechanisms affecting the increase of the risk and for being able to intervene early on and prevent them, is of great interest.

It is important from the perspective of the global assessment of vascular risk to consider VD as a systemic process pathogenically related to endothelial dysfunction, on which there act various risk factors which will determine interindividual expression variability (dyslipidemia, blood hypercoagulability, hyperhomocysteinemia) but which by no means will lead to it being manifested as an organ disease at different levels: cardiovascular, cerebrovascular, peripheral vascular and/or renal level.

The association between coronary and cerebrovascular disease has been partly explained and its study and knowledge has been slow, since the interest for the analysis of the risk factors in cerebrovascular disease has been scarce, which explains why its study began later. Despite the fact that there was a tendency to consider that familial hypercholesterolemia, a disease prototype which indicated a high coronary risk, was not accompanied by ictus, recently conducted meticulous studies demonstrate that what actually happens is that atheromatous cerebrovascular disease develops more slowly than coronary disease, therefore for example in familial hypercholesterolemia, since the onset of the ischemic cardiopathy itself is earlier, it does not allow the development of the cerebrovascular disease in most cases.

According to the foregoing, a thorough stratification must be performed in the evaluation of vascular risk in order to be as objective as possible when evaluating each case. The following are within the large sections which must be analyzed:
a) Metabolic risk: dyslipidemia, hyperhomocysteinemia;
b) Blood hypercoagulability;
c) Endothelial vulnerability; and
d) Hemodynamic status (renin-angiotensin system)

### Dyslipidemia

The predisposition to dyslipidemia or lipid metabolism alteration is also very heterogeneous at molecular level and it is important to evaluate the entire set since among each of the representatives of every genetic polymorphism (presence of allele A or B) which are inherited in an individual, synergies or antagonisms may be established which will determine highly variable and particular risks and therefore vulnerabilities which enable individualizing each case not only in its global assessment, but also in relation to the therapeutic strategy to be used.

### Hyperhomocysteinemia

Homocysteine (HCT) is a demethylated amino acid derived from Methionine and, therefore, an intermediate of the methionine cycle. It is metabolized by remethylation to methionine or by sulfuration to cysteine. For the remethylation, the methionine synthase needs vitamin B12 as a cofactor and folic acid as a substrate. For the transsulfuration, a cystathionine beta-synthase (CBS) and vitamin B6 as a cofactor are required. A defect in the remethylation or the transsulfuration leads to a hyperhomocysteinemia. Various studies have demonstrated that hyperhomocysteinemia, even when it is mild to moderate (greater than 12 nmol/mL) is an independent factor for brain ischemia, myocardial infarction, peripheral artery disease and carotid stenosis. Although the causes coming from the external environment (non-genetic) are important among the causes thereof, there are important genetic alterations to be considered because they determine both the prognosis and the degree of therapeutic response of each case.

The renin-angiotensin system and adrenergic receptors are also factors predisposing to high blood pressure and cardiovascular disease in general.

### Blood Hypercoagulability

According to the classic Virchow's triad, three inter-related factors must be taken into account in the formation of a thrombus: alteration of the blood vessel wall, of the blood flow and of the blood coagulability. It is precisely the alteration of this latter factor which favors the coagulation of the blood, or hypercoagulability or prothrombotic state, which is defined as thrombophilia.

As a general rule, a hypercoagulability state must be suspected in individuals with recurrent episodes of deep vein thromboses, pulmonary embolism, family history of thrombotic events, unusual sites of arterial and venous thrombosis and in children, adolescents or young adults with thrombotic events in general.

### Endothelial Vulnerability

The most evident function of the vascular endothelium is that of maintaining a dilated vascular tone in the exact proportion to preserve the blood pressure at normal values and allow tissue perfusion. This vasodilating function is exerted by the endothelium by means of the synthesis and secretion of relaxation factors such as nitric oxide (NO). Furthermore, the endothelium is an important element for maintaining the balance with platelets and coagulation factors and thus maintaining the fluidity of the blood in what is referred to as homeostatic balance (hemostasis) since the imbalance in one direction or the other will cause hemorrhage or thrombosis.

Most of the factors capable of attacking and damaging the vascular endothelium come from the external environment and one of the most harmful among them is smoking. Nevertheless, there are several genetic polymorphisms which determine a greater vulnerability to this damage and therefore contribute considerably to the general increase of vascular risk. These polymorphisms even worsen the damage which would already be caused by classic non-genetic risk factors themselves such as smoking.

### Oxidative Stress

Oxidative stress is another factor which can also affect to a great extent the better or worse response at endothelial level and at vascular level in general, thus, another important pillar to be considered in the molecular etiopathogenesis of general vascular disease is the degree of defensive potential against oxidative stress.

Ischemic cardiopathy and acute myocardial infarction can be the expression of a process starting with an excess of free radicals, which start the atherosclerotic process by damage in vascular wall, causing the penetration into the subendothelial space of low density lipoproteins (LDL) and therefore into the atherosclerotic plaque.

Various scientific publications analyze the mechanisms of the human organism to produce and at the same time limit the production of reactive oxygen species. An excess of free radicals usually starts the damage of the vascular wall and LDL-cholesterol is involved in this process. A decrease in the incidence of cardiovascular diseases with individual antioxidant supplements has been demonstrated.

### Carcinogenic Risk

This risk relates to the susceptibility with a polygenic and multifactoral basis, not to the monogenic variants of hereditary cancer, therefore adapting each risk to the personal clinical situation and to the family history of each case is recommended.

### Risk of Adverse Reactions to Drugs

The elderly are more prone to suffering from chronic diseases and take a larger amount of drugs than the young, they are therefore more prone to adverse reactions to the drug.

As people get older, the amount of water of the organism decreases. Drugs reach higher concentrations in the elderly. Once in the body, many drugs are dissolved in the fluids of the organism but in these people there is less water for diluting them. Furthermore, the kidneys are much less effective in the excretion of drugs through urine and the liver has a lower capacity for metabolizing them.

For this reason, as people get older, the prescription of suitable doses according to the response of the patient to the drugs becomes more important in order to prevent adverse reactions to such drugs.

It is therefore necessary to develop a method which allows the simultaneous, sensitive, specific and reproducible detection of gene variants associated with pathologies associated with aging (vascular risk, carcinogenic risk, risk of osteoporosis, risk against oxidative stress and risk of adverse reactions to drugs) and which is a tool useful in medicine, particularly in anti-aging medicine. Thus, the clinical and practical translation of this analysis requires the corresponding algorithm integrating the real value of all these gene variants, taking into account the synergies and antagonisms occurring between them, presenting a risk in absolute values which is different depending on the individual analyzed.

The real value of this risk must be considered in the global context of each case taking into account all the classic (non-genetic) risk factors. An objective analysis and unitary vision of a complex and multifactoral disease such as for example a disease associated with aging will only be assured in this way.

### DETAILED DESCRIPTION OF THE INVENTION

The authors of the present invention have developed a method for determining the global genetic risk of a subject to develop a pathology associated with aging. Said method is based on the combination of particular genetic risks of developing common pathologies associated with aging. Said particular genetic risks are determined from the results obtained from the simultaneous genotyping of certain gene variants, particularly of SNPs associated with said pathologies associated with aging and the main objective of which is the use thereof in anti-aging medicine.

Aging is a multifactoral process taking place during the last stage of the life cycle and characterized by the progressive decrease of the functional capacity on all the tissues and organs of the body, and of the consequent ability to adapt to environmental stimuli. Life cycle is a specific characteristic, defined by a maximum potential duration between conception and death and a series of stages during which ontogenetic processes take place: growth, development, maturation and involution. Ontogenetic processes, the sequence in which they occur and their phenotypic expression are genetically programmed and environmentally limited. The sequential and differential expression of one and the same set of genes in specific environments causes the continuum of successive phenotypes corresponding to one and the same individual throughout his or her life cycle. The involutive processes associated with aging are manifested at molecular, cell and functional level with an evident expression in the visible phenotype.

Anti-aging medicine is the part of medicine based on the application of scientific research and of technologies for the prevention and early treatment of diseases related to age or caused by aging, with the objective of lengthening the life expectancy and at the same time improving the quality of life.

For the purpose of achieving an integral and objective assessment of the greater or lower adaptive capacity and capacity of resistance or vulnerability of a subject against most common diseases associated with aging, the inventors of the present invention have developed a method allowing a global assessment of the genetic risk a subject has of suffering from a pathology associated with aging from the calculation of the particular genetic risk of developing certain pathologies associated with aging, particularly, from the calculation of the following particular genetic risks:
1. Vascular risk;
2. Risk of osteoporosis
3. Carcinogenic risk; and
4. Risk of environmental stress and oxidative damage; and, optionally
5. Risk of adverse reactions to drugs.

Thus, the main objective of the present invention is developing an in vitro method for determining the global genetic risk of a subject to develop a pathology associated with aging from a combination of particular genetic risks, particularly, vascular risk, oncogenic risk, risk of osteoporosis, risk of environmental stress and oxidative damage and risk of adverse reactions to drugs.

Therefore, in one aspect, the invention relates to an in vitro method for determining the global genetic risk of a subject to develop a pathology associated with aging from a combination of particular genetic risks, hereinafter method of the invention, comprising:
i) simultaneously genotyping multiple human gene variants present in one or more genes of a subject associated with a pathology associated with aging in a biological sample of said subject;
ii) determining each particular genetic risk; and
iii) determining said global genetic risk according to the value of each particular genetic risk obtained in step ii).

As used in the present description, the term “gene variant” includes mutations, polymorphisms and allelic variants. A genetic variant is found among individuals within populations and among populations within species. In a particular embodiment, the authors of the present invention have selected a total of 69 human gene variants of 49 human genes associated with pathologies associated with aging (Table 1); nevertheless, different additional human gene variants in the same genes or in other human genes, associated with pathologies associated with aging, can be analyzed.

The term “gene mutation” relates to a variation in the nucleotide sequence of a nucleic acid wherein each possible sequence is present in a proportion less than 1% in a population.

The term “polymorphism” relates to a variation in the nucleotide sequence of a nucleic acid wherein each possible sequence is present in a proportion equal to or greater than 1% in a population; in a particular case, when said variation is the nucleotide sequence occurring in single nucleotide (A, C, T or G) it is called SNP.

The terms “allelic variant” or “allele” are used indistinctly in the present description and relate to a polymorphism occurring in one and the same locus in one and the same population.

For the purpose of simultaneously genotyping said human gene variants present in one or more genes of a subject associated with a pathology associated with aging by means of the method of the invention, in a first step the nucleic acid is extracted from a biological sample of the subject to be analyzed.

The extraction of the nucleic acid (e.g., DNA) from a biological sample containing it and coming from a subject, such as a human being, can be carried out by conventional methods optionally using commercial products useful for extracting said nucleic acid. Virtually any biological sample containing nucleic acid can be used to put the invention into practice; by way of a non-limiting illustration, said biological sample can be a sample of blood, saliva, plasma, serum, secretions, tissue, etc.

Once the nucleic acid is obtained, those regions of said nucleic acid containing the gene variants to be identified are amplified. As has been previously mentioned, as used in this description, the term “gene variant” includes polymorphisms (e.g., SNPs), mutations and allelic variants. To amplify the regions of nucleic acid containing the gene variants to be identified, specific oligonucleotide primers amplifying the genome fragments which can contain said gene variants are used. Said oligonucleotide primers are described in detail below, they form part of the present invention and form an additional aspect thereof. If desired, said amplification products can be optionally labeled during the amplification reaction to obtain labeled amplification products containing the gene variants to be identified.

Thus, the DNA regions containing the gene variants to be identified (target DNA regions) are subjected to an amplification reaction to obtain amplification products containing the gene variants to be identified. Although any technique or method allowing the amplification of all the DNA sequences containing the gene variants to be identified can be used, in a particular embodiment, said sequences are amplified by means of a multiplex amplification, which allows simultaneously genotyping said human gene variants to be identified present in one or more genes.

To perform a multiplex amplification, the use of pairs of oligonucleotide primers or primers capable of amplifying said target DNA regions containing the gene variants to be identified as has been previously explained is required. Virtually any pair of oligonucleotide primers allowing the specific amplification of said target DNA regions can be used, preferably, pairs of oligonucleotide primers allowing said amplification in the smallest possible number of amplification reactions. Thus, using the suitable pairs of oligonucleotide primers and conditions, all the target DNA regions necessary for the genotyping of said gene variants to be analyzed can be amplified with the smallest possible number of reactions. In a particular embodiment, said oligonucleotide primers are selected from the oligonucleotide primers identified as SEQ ID NO: 277-278, SEQ ID NO: 285-319, SEQ ID NO: 321-326, SEQ ID NO: 333-340, SEQ ID NO: 343-356, SEQ ID NO: 359-362, SEQ ID NO: 364, SEQ ID NO: 367, SEQ ID NO: 369-371, SEQ ID NO: 374, SEQ ID NO: 377-381, SEQ ID NO: 383, SEQ ID NO: 385-402 and SEQ ID NO: 404-414.

Once the DNA sequences containing the gene variants to be identified have been amplified, the method of the invention comprises the step of simultaneously genotyping multiple human gene variants present in one or more genes of a subject associated with a pathology associated with aging. In a particular embodiment of the invention, said step of simultaneous genotyping is performed by means of an analysis with DNA-chips, for example, using a suitable DNA-chip, such as the DNA-chip provided by this invention (DNA-chip of the invention, the features of which are mentioned below), i.e., by hybridization with specific probes for said human gene variants. Additionally or alternatively, said genotyping can be performed by means of the gene sequencing of said amplification products.

Thus, if desired, during the amplification reaction, the amplification products can be labeled for the purpose of being able to subsequently detect the hybridization between the probes present in the DNA-chip of the invention, immobilized in the support, and the target DNA fragments containing the gene variants to be detected. The amplification products can be labeled by conventional methods, for example, incorporating a labeled nucleotide during the amplification reaction or using labeled primers. Said labeling can be direct, for which fluorophores, for example, Cy3, Cy5, fluorescein, Alexa, etc., enzymes, for example, alkaline phosphatase, peroxidase, etc., radioactive isotopes, for example, 33P, 125I, etc., or any other marker known by the person skilled in the art can be used. Alternatively, said labeling can be indirect by means of using chemical methods, enzymatic methods, etc.; by way of illustration, the amplification product can incorporate a member of a specific binding pair, for example, avidin or streptavidin conjugated with a fluorochrome (marker), and the probe binds to the other member of the specific binding pair, for example, biotin (indicator), the reading being performed by means of fluorometry, etc., or the amplification product can incorporate a member of a specific binding pair, for example, an anti-digoxigenin antibody conjugated with an enzyme (marker), and the probe binds to the other member of the specific binding pair, for example, digoxigenin (indicator), etc., the substrate of the enzyme being transformed into a luminescent or fluorescent product and the reading being performed by means of chemiluminescence, fluorometry, etc.

In a particular embodiment, the amplification product is labeled by means of using a nucleotide labeled directly or indirectly with one or more fluorophores. In another particular embodiment, the amplification product is labeled by means of using primers labeled directly or indirectly with one or more fluorophores.

In a particular case, said amplification products are subjected to a fragmentation reaction to obtain fragmentation products containing the gene variants to be identified, and, in the event that said amplification products were not previously labeled in the amplification step, said fragmentation products containing the gene variants to be identified can be labeled.

The optionally labeled amplification products are subsequently subjected to fragmentation reaction for the purpose of increasing the efficiency of the subsequent hybridization, fragmentation products containing the gene variants to be identified thus being obtained. The fragmentation of the amplification products can be carried out by any conventional method, for example, contacting the amplification products with a DNAse.

In the event that the amplification products were not previously labeled during the amplification reaction, and in the event that after the hybridization process, an amplification or ligation reaction is not carried out directly in the support, the products resulting from the fragmentation reaction (fragmentation products) are subjected to a labeling which is either direct, using, for example, fluorophores, enzymes, radioactive isotopes, etc. or indirect, using, for example, specific binding pairs incorporating fluorophores, enzymes, etc., by means of conventional methods. In a particular embodiment, the amplification products have not been previously labeled during the amplification reaction, and the fragmentation products are subjected to a direct or indirect labeling with one or several markers, for example, one or several fluorophores, although other markers known by persons skilled in the art can be used.

The fragmentation products are then contacted with probes capable of detecting the corresponding gene variants under conditions allowing the hybridization between said fragmentation products and said probes. Said probes are deposited on a solid support following a predetermined arrangement, forming a DNA-chip (DNA-chip of the invention), the design and development of which must comply with a series of requirements to be able to used in the method of the invention in relation to the design of the probes, the number of probes to be deposited per gene variant to be detected, the number of probe replicas to be deposited, the distribution of the probes on the support, etc. The typical features of said DNA-chip of the invention and of said probes are described in detail below.

The hybridization of the fragmentation products with the probes capable of detecting the corresponding gene variants deposited on a support (DNA-chip of the invention) is carried out by conventional methods using conventional devices. In a particular embodiment, the hybridization is carried out in an automatic hybridization station. To carry out the hybridization, the fragmentation products are contacted with said probes (DNA-chip of the invention) under conditions allowing the hybridization between said fragmentation products and said probes. Stable hybridization conditions allow establishing the strand and the suitable length of the probes for the purpose of maximizing the discrimination, as mentioned below.

Once the hybridization process has ended, the image is captured and quantified. To that end, the image of the hybridized and developed DNA-chip is collected with a suitable device, for example, a scanner, the absolute fluorescence values of each probe as well as the background noise then being quantified. Therefore, in a particular embodiment, after the hybridization, or after the post-hybridization ligation or amplification reactions, the hybridized and developed DNA-chip is introduced in a scanner where it is subjected to a scanning to quantify the intensity of the labeling at the points in which the hybridization has occurred. Although virtually any scanner can be used, in a particular embodiment, said scanner is a confocal fluorescence scanner. In this case, the DNA-chip is introduced in the scanner and the signal emitted by the labeling upon being excited by a laser is scanned, the intensity of the points in which the hybridization has occurred being quantified. In a particular embodiment, said scanner is a white light scanner. Illustrative non-limiting examples of scanners which can be used according to the present invention are Axon, Agilent, Perkin Elmer scanners, etc.

The data is then analyzed and interpreted, which can be carried out by means of using any suitable genotyping software, such as the genotyping software referred to in Example 1, which uses the functions described in section 1.3.5 of said Example 1, and by means of using functions developed by the inventors to calculate the corresponding particular genetic risks and, from them, the global genetic risk, as described in detail below.

The analysis of the data and its interpretation is generally carried out by means of using computer programs (software). The inventors have developed a sequential method for processing and interpreting the experimental data generated by the DNA-chip of the invention which allows detecting each of the gene variants with sensitivity, specificity and reproducibility, and calculating the values of the corresponding particular genetic risks and, from them, the global genetic risk, by means of algorithms according to the genotype of the processed sample. The algorithms and computer software developed by the inventors allow facilitating and automating the application of the method of the invention.

The execution of the algorithms and computer software developed by the inventors to sequentially process and interpret the experimental data generated by the DNA-chip of the invention comprises performing a series of steps for characterizing each of the gene variants of interest, specifically:
firstly, the own background noise of the absolute intensity values of all the probes is subtracted therefrom;
the replicas corresponding to each of the 4 probes used to characterize each gene variant are then grouped;
the mean intensity value for each of the 4 probes is calculated using the bounded mean of the replicas to eliminate the aberrant points;
once the mean intensity values for each of the probes are known, Ratio 1 and Ratio 2 are calculated, wherein:
   Ratio 1 is the proportion of the bounded mean of the intensities of the 10, 8 or 6 replicas of the probe 1 detecting gene variant A divided by the bounded mean of the 10, 8 or 6 replicas of the probe 1 detecting gene variant A plus the bounded mean of the 10, 8 or 6 replicas of the probe 2 detecting gene variant B and can be calculated by means of the equation:

$$Ratio 1 = \frac{Mean intensity probe 1}{Mean intensity probe 1 + Mean intensity probe 2}$$

Ratio 2 is the proportion of the bounded mean of the intensities of the 10, 8 or 6 replicas of the probe 3 detecting gene variant A divided by the bounded mean of the 10, 8 or 6 replicas of the probe 3 detecting gene variant A plus the bounded mean of the 10, 8 or 6 replicas of the probe 4 detecting gene variant B and can be calculated by means of the equation:

$$Ratio 2 = \frac{Mean intensity probe 3}{Mean intensity probe 3 + Mean intensity probe 4}$$

said ratios (Ratio 1 and Ratio 2) are substituted in three linear functions, which characterize each of the three possible genotypes:

| | |
|---|---|
| AA | Function 1 |
| AB | Function 2 |
| BB | Function 3 |

wherein
AA represents the genotype of a homozygous subject for gene variant A;
AB represents the genotype of a heterozygous subject for gene variants A and B;
BB represents the genotype of a homozygous subject for gene variant B;
Function 1 is the Linear Function characterizing the patients with genotype AA and consists of a linear combination of the variables Ratio 1 and Ratio 2;
Function 2 is the Linear Function for genotype AB and consists of a linear combination of the variables Ratio 1 and Ratio 2;
Function 3 is the Linear Function for genotype BB and consists of a linear combination of the variables Ratio 1 and Ratio 2;
wherein the linear combinations are formed by constants and cofactors accompanying the variables Ratio 1 and Ratio 2; and the function having a greater absolute value determines the genotype presented by the patient for the gene variant analyzed.

These ratios serve as variables for classifying the three groups for generating the linear functions.

In another particular embodiment of the invention, the genotyping of the multiple human gene variants or polymorphisms present in one or more genes of a subject associated with a pathology associated with aging in said biological sample is performed by gene sequencing.

Once said gene variants have been genotyped, each particular genetic risk is determined. Depending on whether the particular genetic risk to be calculated is formed by a combination of partial particular risks, said particular genetic risk is calculated applying different functions, as described below.

In a particular embodiment, the determination (calculation) of the particular genetic risk (step ii) of the method of the invention) comprises:
i) grouping the results obtained relating to each particular genetic risk of developing a pathology associated with aging;
ii) standardizing the value of each genotype of each gene variant analyzed;
iii) calculating each particular genetic risk such that:
   iiia) when said particular genetic risk is not formed by a combination of partial particular risks, said particular genetic risk is calculated by means of equation [1]:

$$PGR = \frac{{\sum }_{i = 1}^{n} xi}{{\sum }_{i = 1}^{n} Lsi} \qquad [ 1 ]$$

where
   PGR represents the particular genetic risk to be calculated;
   xᵢ represents the standardized value of the genotype characterized for a gene variant in a sample, in relation to the particular genetic risk to be calculated;
   Lsᵢ represents the value of the upper limit of the range of standardized values assigned to each gene variant, in relation to the particular genetic risk to be calculated; and
   n is the number of gene variants analyzed in relation to the particular genetic risk to be calculated; or, alternatively,
iiib) when said particular genetic risk is formed by a combination of partial particular risks, said particular genetic risk is calculated by means of equation [2]:

$$PGR = \frac{{\sum }_{i = 1}^{n} PPGRi}{no . PPGR} \qquad [ 2 ]$$

where
   PGR represents the particular genetic risk to be calculated;
   PPGRi represents the value calculated for each partial particular genetic risk which, in combination with other partial particular genetic risks, forms the particular genetic risk to be calculated, wherein said PPGRi is calculated by means of equation [3]:

$$PPGRi = \frac{{\sum }_{i = 1}^{n} xi}{{\sum }_{i = 1}^{n} Lsi} \qquad [ 3 ]$$

where
   PPGRi has the previously mentioned meaning;
   xᵢ represents the standardized value of the genotype characterized for a gene variant in a sample, in relation to the partial particular genetic risk to be calculated;
   Lsᵢ represents the value of the upper limit of the range of standardized values assigned to each gene variant, in relation to the partial particular genetic risk to be calculated; and
   n is the number of gene variants analyzed in relation to the partial particular genetic risk to be calculated; and
no.PPGR is the number of partial particular genetic risks analyzed in relation to the partial particular genetic risk to be calculated.

Thus, in a first step, after the genotyping of the human gene variants, said variants are grouped by particular genetic risks and partial particular genetic risks, i.e., the results of the analysis of the gene variants [mutations, polymorphisms (e.g., SNPs), allelic variants, etc.] are grouped by particular genetic risks and, where appropriate, by partial particular genetic risks, for the purpose of calculating the particular genetic risk of each pathology associated with aging. In a particular embodiment of the invention, said particular genetic risk is selected from the group formed by particular genetic risk associated with suffering from vascular disease (vascular risk), particular genetic risk associated with osteoporosis, particular genetic risk associated with carcinogenesis and particular genetic risk associated with environmental stress and oxidative damage. Likewise, in a particular embodiment, said vascular risk is determined according to the partial particular genetic risks selected from the group formed by partial particular genetic risk associated with lipid metabolism, partial particular genetic risk associated with thrombosis, partial particular genetic risk associated with ictus, partial particular genetic risk associated with high blood pressure and partial particular genetic risk associated with endothelial vulnerability.

Subsequently, in a second step, the value of each genotype of each gene variant is standardized or scored. In this sense, said values will be comprised in a range of standardized values, in which the genotype or genotypes of the highest risk of suffering from a certain pathology will comprise the value of the upper limit of said range of values, and the genotype or genotypes of the lowest risk of suffering from a certain pathology will comprise the value of the lower limit of said range of values. Thus, according to the genotype present in the sample analyzed, a corresponding standardized value is assigned to said genotype. The particular genetic risks are then calculated according to equation [1] or [2] depending on whether the particular genetic risk to be calculated is formed by a combination of partial particular risks. In a particular embodiment, the particular genetic risk associated with osteoporosis, the particular genetic risk associated with carcinogenesis and the particular genetic risk associated with environmental stress and oxidative damage are calculated by means of equation [1], whereas in another particular embodiment, the vascular risk is determined using equation [2] according to the partial particular genetic risks selected from the group formed by partial particular genetic risk associated with lipid metabolism, partial particular genetic risk associated with thrombosis, partial particular genetic risk associated with ictus, partial particular genetic risk associated with high blood pressure and partial particular genetic risk associated with endothelial vulnerability, such that, in this case, the particular genetic risk is calculated according to the values of the different partial particular genetic risks analyzed as shown in Example 1 attached to the present description.

In any case, the person skilled in the art will understand that, depending on whether partial particular genetic risks are used to determine the particular genetic risks, he will use the suitable equation in each case.

### Vascular Risk

The particular genetic risk associated with suffering from vascular risk or suffering from a vascular disease (VD) (vascular risk) is altogether one of the main causes of mortality and morbidity virtually everywhere in the world, therefore the development of models for predicting the risk of suffering from this type of disease, both for attempting to know the possible mechanisms affecting the increase of the risk and for being able to intervene early on and prevent them, is of great interest.

In this sense, the research of the molecular bases of VD has indicated genes which are involved in each of the sections and which confer susceptibility to this disease.

On one hand, the genes regulating everything related to lipid metabolism have been considered. In addition, it is also known that another of the conditions predisposing to VD is in the tendency for thrombus formation, therefore the inventors have searched for polymorphisms of risk among the genes involved in the coagulation cascade and the fibrinolytic system. On the other hand, the method of the invention analyzes genetic risk factors among those genes with influence at the level of structural and functional preservation of the vascular endothelium and among the genes involved in the defense mechanisms against oxidative stress.

### Partial Particular Genetic Risk Related to the Integrity of the Lipid Metabolism (Lipid Metabolism)

The term dyslipidemia relates to various pathologic conditions the only common element of which is a lipid metabolism alteration, with its subsequent alteration of the concentrations of lipids and lipoproteins in the blood. The predisposition to dyslipidemia is very heterogeneous at molecular level and it is important to evaluate the entire set since among each of the alleles or variants of every genetic polymorphism which are inherited in an individual, synergies or antagonisms may be established which will determine highly variable and particular risks and therefore vulnerabilities which enable individualizing each case not only in its global assessment, but also in relation to the therapeutic strategy to be used.

### Partial Particular Genetic Risk of Thrombosis

According to the classic Virchow's triad, three inter-related factors must be taken into account in the formation of a thrombus: alteration of the blood vessel wall, of the blood flow and of the blood coagulability. It is precisely the alteration of this latter factor which favors the coagulation of the blood, or hypercoagulability or prothrombotic state, which is defined as thrombophilia.

As a general rule, a hypercoagulability state must be suspected in individuals with recurrent episodes of deep vein thromboses, pulmonary embolism, family history of thrombotic events, unusual sites of arterial and venous thrombosis and in children, adolescents or young adults with thrombotic events in general.

This section includes several gene variations which can act in a synergic manner (enhancing the pathogenic effect) or antagonistic manner (providing a natural compensation).

### Partial Particular Genetic Risk of High Blood Pressure

In this case, the state at hemodynamic level is analyzed, specifically assessing the renin-angiotensin system and the adrenergic receptors which basically predispose to high blood pressure and cardiovascular disease in general. The assessment thereof would also allow objectively defining, on molecular bases, the most effective therapeutic strategy to achieve the control in each case.

### Partial Particular Genetic Risk of Endothelial Vulnerability

The most evident function of the vascular endothelium is that of maintaining a dilated vascular tone in the exact proportion to preserve the blood pressure at normal values and allow tissue perfusion. This vasodilating function is exerted by the endothelium by means of the synthesis and secretion of relaxation factors such as nitric oxide (NO). Furthermore, the endothelium is an important element for maintaining the balance with platelets and coagulation factors and thus maintaining the fluidity of the blood in what is referred to as homeostatic balance (hemostasis) since the imbalance in one direction or the other will cause hemorrhage or thrombosis.

Most of the factors capable of attacking and damaging the endothelium come from the external environment and one of the most harmful among them is smoking. Nevertheless, there are several gene variations which determine a greater vulnerability to this damage and therefore contribute considerably to the general increase of vascular risk. These gene variations even worsen the damage which would already be caused by classic non-genetic risk factors themselves such as smoking.

In addition, it is known that homocysteine (HCT), a demethylated amino acid derived from methionine and, therefore, an intermediate of the methionine cycle, is metabolized by remethylation to methionine or by sulfuration to cysteine. For the remethylation, the methionine synthase needs vitamin B12 as a cofactor and folic acid as a substrate. For the transsulfuration, a cystathionine beta-synthase (CBS) and vitamin B6 as a cofactor are required. A defect in the remethylation or the transsulfuration leads to a hyperhomocysteinemia. Various studies have demonstrated that hyperhomocysteinemia, even when it is mild to moderate (greater than 12 nmol/mL) is an independent factor for brain ischemia, myocardial infarction, peripheral artery disease and carotid stenosis and it is therefore important to take it into account in the assessment of vascular risk. Although the causes coming from the external environment (non-genetic) are important among the causes thereof, there are important genetic alterations to be considered because they determine both the prognosis and the degree of therapeutic response of each case.

Oxidative stress is another factor which can also affect our better or worse response at endothelial level and at vascular level in general. For this reason, this factor can be considered in the molecular etiopathogenesis of general vascular disease, and this is none other than the degree of defensive potential against oxidative stress.

Ischemic cardiopathy and acute myocardial infarction can be the expression of a process starting with an excess of free radicals, which start the atherosclerotic process by damage in vascular wall, causing the penetration into the subendothelial space of low density lipoproteins (LDL) and therefore into the atherosclerotic plaque. Various scientific publications analyze the mechanisms of the human organism to produce and at the same time limit the production of reactive oxygen species. An excess of free radicals usually starts the damage of the vascular wall and LDL-cholesterol is involved in this process. A decrease in the incidence of cardiovascular diseases with individual antioxidant supplements has been demonstrated.

Once each particular genetic risk has been determined, the global genetic risk is determined by applying suitable functions. In a particular embodiment, the determination (calculation) of the global genetic risk is carried out by means of equation [4]:

$$GGR = \frac{\sum PGR}{n} \qquad [ 4 ]$$

where
   GGR represents the global genetic risk to be calculated;
   PGR represents the value calculated for each particular genetic risk analyzed in relation to the global genetic risk to be calculated, and is calculated by means of the previously described equations [1] or [2]; and
   n is the number of particular genetic risks analyzed in relation to the global genetic risk to be calculated.

Merely by way of a non-limiting illustration, the method provided by this invention for determining the global genetic risk a subject has of developing a pathology associated with aging comprises calculating or determining the following particular genetic risks:
1. Particular genetic risk associated with suffering from vascular disease (vascular risk);
2. Particular genetic risk associated with osteoporosis (risk of osteoporosis);
3. Particular genetic risk associated with carcinogenesis (carcinogenic risk); and
4. Particular genetic risk associated with environmental stress and oxidative damage.

Likewise, in a particular embodiment, said vascular risk is determined according to the partial particular genetic risks selected from the group formed by partial particular genetic risk associated with lipid metabolism, partial particular genetic risk associated with thrombosis, partial particular genetic risk associated with ictus, partial particular genetic risk associated with high blood pressure and partial particular genetic risk associated with endothelial vulnerability.

More specifically, in a particular embodiment, said partial particular genetic risk associated with lipid metabolism is determined according to the gene variants selected from the group formed by −75 G>A of the APOA1 gene, Arg3480Trp of the APOB gene, Arg3500Gln of the APOB gene, Arg3531Cys of the APOB gene, Cys112Arg of the APOE gene, Arg158Cys of the APOE gene, Arg451Gln of the CETP gene, TaqIB B1>B2 of the CETP gene, Gln192Arg of the PON1 gene, Gly595Ala of the SREBF2 gene, Leu7Pro of the NPY gene and combinations thereof.

In another particular embodiment, said particular genetic risk associated with thrombosis is determined according to the gene variants selected from the group formed by 4G>5G of the PAI1 gene, Leu33Pro of the ITGB3 gene, 20210 G>A of the FII gene, Arg506Gln of the FV Leiden gene, Val34Leu of the F13A1 gene, Ala222Val of the MTHFR gene, 833 T>C of the CBS gene, 844ins68 of the CBS gene, −455 G>A of the FGB gene and combinations thereof.

In another particular embodiment, said particular genetic risk associated with ictus is determined according to the gene variants selected from the group formed by 4G>5G of the PAI1 gene, Leu33Pro of the ITGB3 gene, 20210 G>A of the FII gene, Arg506Gln of the FV Leiden gene, Val34Leu of the F13A1 gene and combinations thereof.

In another particular embodiment, said particular genetic risk associated with high blood pressure is determined according to the gene variants selected from the group formed by Gly389Arg of the ADRB1 gene; Gln27Glu of the ADRB2 gene, Gly16Arg of the ADRB2 gene, Met235Thr of the AGT gene, 1166 A>C of the AGTR1 gene, 393 T>C (Ile131Ile) of the GNAS gene, 825 C>T (Ser275Ser) of the GNB3 gene, intron 16 ins/del of the ACE gene, Trp64Arg of the ADRB3 gene and combinations thereof.

In another particular embodiment, said particular genetic risk associated with endothelial vulnerability is determined according to the gene variants selected from the group formed by 5A>6A of the MMP3 gene, −786 T>C of the NOS3 gene, Glu298Asp of the NOS3 gene, Ala222Val of the MTHFR gene, 833 T>C of the CBS gene, 844ins68 of the CBS gene, Pro319Ser of the GJA4 gene and combinations thereof.

In addition, in a particular embodiment of the invention, said particular genetic risk associated with osteoporosis is determined according to the gene variants selected from the group formed by 1546 G>T of the COL1A1 gene, IVS1-397 T>C p>P (PvuII) of the ESR1 gene, b>B of the VDR gene and combinations thereof.

In another particular embodiment, said particular genetic risk associated with carcinogenesis is determined according to the gene variants selected from the group formed by −34 A>G of the CYP17A1 gene, Ile462Val of the CYP1A1 gene, T3801C of the CYP1A1 gene, Leu432Val of the CYP1B1 gene, Allele*4 (Asn453Ser) of the CYP1B1 gene, 1558 C>T of the CYP19A1 gene, Val158Met (Allele*2) of the COMT gene, 331 G>A of the PGR gene, IVS1-397 T>C p>P (PvuII) of the ESR1 gene, b>B of the VDR gene, Ala49Thr of the SRD5A2 gene, Val89Leu of the SRD5A2 gene, Ala541Thr of the ELAC2 gene and combinations thereof.

In a particular embodiment, said particular genetic risk associated with environmental stress and oxidative damage is determined according to the gene variants selected from the group formed by Cys326Ser of the OGG1 gene, Ala16Val of the SOD2 gene, Arg213H is of the SULT1A1 gene, present>null GSTM1, present>null GSTT1, Ile105Val of the GSTP1 gene, Ala114Val of the GSTP1 gene, Val158Met (Allele*2) of the COMT gene, −174 C>G of the IL6 gene, −1082 G>A of the IL10 gene, R64Q of the NAT2 gene, 282 C>T (Y94Y) of the NAT2 gene, I114T of the NAT2 gene, 481C>T (L161L) of the NAT2 gene, R197Q of the NAT2 gene, K268R of the NAT2 gene, G286E of the NAT2 gene and combinations thereof.

If desired, the method of the invention further comprises evaluating or determining the particular genetic risk associated with the response to drugs, i.e., the particular genetic risk of suffering from adverse reactions to drugs.

In a particular embodiment, said particular genetic risk associated with the response to drugs is determined according to the gene variants selected from the group formed by R64Q, 282 C>T (Y94Y), I114T, 481C>T (L161L), R197Q, K268R and G286E of the NAT2 gene; Arg144Cys (allele*2) and Ile359Leu (allele*3) of the CYP2C9 gene; 681 G>A (Pro227Pro) (allele*2) of the CYP2C19 gene; 2549 A>del (allele*3), 1847 G>A (allele*4) and 1707 del>T (allele*6) of the CYP2D6 gene; and combinations thereof.

Therefore, in a particular embodiment, the method of the invention comprises simultaneously genotyping multiple human gene variants or polymorphisms present in one or more genes of a subject associated with a pathology associated with aging in a biological sample of said subject, wherein said gene variant [mutation, polymorphism (e.g., SNP) or allelic variation] to be genotyped is selected from the group formed by the intron ins/del polymorphism of the ACE gene; the Gly389Arg polymorphism of the ADRB1 gene; the Gln27Glu and Gly16Arg polymorphisms of the ADRB2 gene; the Trp64Arg polymorphism of the ADRB3 gene; the Met235Thr polymorphism of the AGT gene; the 1166 A>C polymorphism of the AGTR1 gene; the −75 G>A polymorphism of the APOA1 gene; the Arg3480Trp, Arg3500Gln and Arg3531Cys polymorphisms of the APOB gene; the Cys112Arg and Arg158Cys polymorphisms of the APOE gene; the 833 T>C and 844ins68 polymorphisms of the CBS gene; the TaqIB B1>B2 and Arg451Gln polymorphisms of the CETP gene; the 1546 G>T polymorphism of the COL1A1 gene; the Val158Met (Allele*2) polymorphism of the COMT gene; the −34 A>G polymorphism of the CYP17A1 gene; the 1558 C>T polymorphism of the CYP19A1 gene; the Ile462Val and T3801C polymorphism of the CYP1A1 gene; the Leu432Val and Allele*4 (Asn453Ser) polymorphism of the CYP1B1 gene; the Arg144Cys (allele*2) and Ile359Leu (allele*3) polymorphism of the CYP2C9 gene; the 681 G>A (Pro227Pro) (allele*2) polymorphism of the CYP2C19 gene; the 2549 A>del (allele*3), 1847 G>A (allele*4) and 1707 del>T (allele*6) polymorphism of the CYP2D6 gene; the Ala541Thr polymorphism of the ELAC2 gene; the IVS1-397 T>C p>P (PvuII) polymorphism of the ESR1 gene; the Val34Leu polymorphism of the F13A1 gene; the −455 G>A polymorphism of the FGB gene; the 20210 G>A polymorphism of the FII gene; the Arg506Gln polymorphism of the FV Leiden gene; the Pro319Ser polymorphism of the GJA4 gene; the 393 T>C (Ile131Ile) polymorphism of the GNAS gene; the 825 C>T (Ser275Ser) polymorphism of the GNB3 gene; the present>null GSTM1 polymorphism; the Ile105Val and Ala114Val polymorphisms of the GSTP1 gene; the present>null GSTT1 polymorphism; the −174 C>G polymorphism of the IL6 gene; the −1082 G>A polymorphism of the IL10 gene; the Leu33Pro polymorphism of the ITGB3 gene; the 5A>6A polymorphism of the MMP3 gene; the Ala222Val polymorphism of the MTHFR gene; the R64Q, 282 C>T (Y94Y), I114T, 481C>T (L161L), R197Q, K268R and G286E polymorphisms of the NAT2 gene; the −786 T>C and Glu298Asp polymorphisms of the NOS3 gene; the Leu7Pro polymorphism of the NPY gene; the Cys326Ser polymorphism of the OGG1 gene; the 4G>5G polymorphism of the PAI1 gene; the 331 G>A polymorphism of the PGR gene; the Gln192Arg polymorphism of the PON1 gene; the Ala16Val polymorphism of the SOD2 gene; the Ala49Thr and Val89Leu polymorphisms of the SRD5A2 gene; the Gly595Ala polymorphism of the SREBF2 gene; the Arg213H is polymorphism of the SULT1A1 gene; the b>B polymorphism of the VDR gene; and combinations thereof.

Likewise, if desired, the method of the invention further comprises genotyping one or more additional gene variants associated with pathologies associated with aging.

The method of the invention is therefore an extracorporeal in vitro method for the simultaneous, sensitive, specific and reproducible genotyping of multiple human gene variants present in different genes, associated with pathologies associated with aging. The method of the invention allows identifying changes of nucleotides, insertions, deletions, etc. and determining the genotype of a subject for the gene variants related to pathologies associated with aging analyzed.

To put the method of the invention into practice, a genotyping DNA-chip useful for detecting said gene variants has been developed.

Therefore, in another aspect, the invention relates to a DNA-chip, hereinafter DNA-chip of the invention, comprising a support on which there is deposited a plurality of probes useful for detecting human gene variants present in one or more genes associated with pathologies associated with aging. In a particular embodiment, said probes are selected from the group formed by the probes identified as SEQ ID NO: 1-13, SEQ ID NO: 15, SEQ ID NO: 17-44, SEQ ID NO: 53-128, SEQ ID NO: 130, SEQ ID NO: 132-172, SEQ ID NO: 181-200, SEQ ID NO: 202, SEQ ID NO: 204, SEQ ID NO: 206, SEQ ID NO: 208, SEQ ID NO: 210, SEQ ID NO: 212, SEQ ID NO: 222, and SEQ ID NO: 224-276 (see section 1.1 of Example 1 attached to the description).

The DNA-chip of the invention comprises a support on which there is deposited a plurality of probes useful for detecting human gene variants present in one or more genes associated with pathologies associated with aging. For every gene variant, the DNA chip of the invention comprises 4 probes, of which 2 probes detect a first gene variant and the other 2 detect a second gene variant, wherein the number of replicas of each of said probes is 10, 8 or 6 replicas and the two probes do not have to be identical. Said probes are deposited following a certain pattern and distributed homogeneously between the 2 areas forming the DNA-chip but not grouped by gene variant to be detected, i.e., they are distributed along the length and width of the chip and furthermore they are not grouped within one and the same gene variant.

The DNA-chip of the invention can also contain, if desired, oligonucleotides deposited on the support useful as positive and negative controls of the amplification and/or hybridization reactions.

For the present DNA-chip to allow the simultaneous, sensitive, specific and reproducible detection of gene variants, be completely effective and actually be a useful tool in anti-aging medicine, the clinical and practical translation of this analysis requires the corresponding algorithm integrating the real value of all these polymorphisms, taking into account the synergies and antagonisms occurring between them, presenting a risk in absolute values which is always different depending on the individual analyzed. The real value of this risk must be considered in the global context of each case taking into account all the classic (non-genetic) risk factors. An objective analysis and unitary vision of a complex and multifactoral disease such as for example vascular disease will only be assured in this way.

For the purpose of maximally decreasing the rate of false positives and negatives, the DNA-chip of the invention comprises two pairs of probes for detecting each genetic variation. Each pair of probes is formed by a specific probe for the detection of a genetic variation (e.g., allele A) and by another probe designed for the detection of another genetic variation (e.g., allele B). In the case of point mutations, the base differing between allele A and B (base to be interrogated) is placed in the central position of the probe, which assured the maximum specificity in the hybridization. In the case of insertions, duplications or deletions, there are several bases which can be interrogated. However, the design becomes completely equivalent considering as the central position the first nucleotide which is different in the normal sequence with respect to the mutated sequence.

In a particular embodiment, the DNA-chip of the invention comprises 10 replicas of each of the 4 probes used to detect each genetic variation; in another particular embodiment, the DNA-chip of the invention comprises 8 replicas of each of the 4 probes used to detect each genetic variation; and, in another particular embodiment, the DNA-chip of the invention comprises 6 replicas of each of the 4 probes used to detect each genetic variation.

The arrangement (placement) of the probes in the support is predetermined. In a particular embodiment, although the probes deposited on the support maintain a predetermined arrangement, they are not grouped by genetic variation but rather they have a random distribution, which, if desired, can always be the same.

The capacity of the specific probes of gene variants to discriminate between the gene variants (e.g., allele A and allele B) depend on the hybridization conditions, on the sequence flanking the mutation and on the secondary structure of the sequence in which the polymorphism is to be detected. Stable hybridization conditions allow establishing the strand and the suitable length of the probes for the purpose of maximizing the discrimination. Starting from probes of 25 nucleotides detecting a genetic variation (e.g., allele A) and another genetic variation (e.g., allele B) in both strands (sense strand and antisense strand), a mean of 8 experimentally assayed probes is required in order to be left with the two definitive pairs.

In a particular embodiment, for every genetic variation to be detected by means of the DNA-chip of the invention, the designed probes interrogate both strands, with lengths typically comprised between 19 and 27 nucleotides, and the hybridization temperature varies between 75° C. and 85° C.

Table 1 (Example 1) includes a list of gene variants associated with pathologies associated with aging; nevertheless, probes allowing the identification of other gene variants associated with said diseases can be incorporated in the DNA-chips of the invention.

As has been mentioned previously, the DNA-chip of the invention can optionally contain oligonucleotides deposited on the support useful as positive and negative controls of the amplification and/or hybridization reactions. In a particular embodiment, the DNA-chip of the invention comprises oligonucleotides deposited on the support useful as positive and negative controls of the hybridization reactions. In general, each of the sub-arrays forming a DNA-chip is flanked by external hybridization controls which allow easily locating the points on the support. Although with the same sequence, the DNA-chip has two external hybridization controls labeled, for example, with a fluorophore (e.g., Cy3, Cy5, etc.), which serve to evaluate the hybridization quality in both channels. In a particular embodiment, the nucleotide sequence of the external control is the one identified in SEQ ID NO: 415 (CEH), and the sequences of the oligonucleotides for the detection thereof are those identified in SEQ ID NO: 416 and SEQ ID NO: 417.

The support on which the plurality of probes is deposited can be any solid surface on which the oligonucleotides can be bound. Virtually any support on which an oligonucleotide used in the production of DNA-chips can be bound or immobilized can be used to put this invention into practice. By way of illustration, said support can be a non-porous support, for example, a support made of glass, silicon, plastic, etc., or a porous support, for example, membranes (nylon, nitrocellulose, etc.), microparticles, etc. In a particular embodiment, said support is a glass slide.

The probes are immobilized (bound) on the support using conventional techniques for immobilizing oligonucleotides on the surface of the supports. Said techniques depend, among other factors, on the nature of the support used [porous (membranes, microparticles, etc.) or non-porous (glass, plastic, silicon, etc.)]. In general, the probes can be immobilized on the support by means of using non-covalent immobilization techniques or by means of using immobilization techniques based on the covalent binding of the probes to the surface of the support by means of chemical processes.

The preparation of non-porous supports (e.g., glass, silicon, plastic, etc.) generally requires a prior treatment with reactive groups (e.g., amino, aldehyde, etc.) or coating the surface of the support with a member of a specific binding pair (e.g., avidin, streptavidin, etc.). Likewise, it is generally convenient to previously activate the probes to be immobilized by means of thiol, amino groups, etc., or biotin, etc., for the purpose of achieving a specific immobilization of the probes on the support.

The immobilization of the probes on the support can be carried out by conventional methods, for example, by means of techniques based on the synthesis in situ of the probes on the support itself (e.g., photolithography, direct chemical synthesis, etc.), or by means of techniques based on the use of robotized arms depositing the corresponding pre-synthesized probe (printing without contact, printing by contact, etc.), etc.

The arrangement (placement) of the probes in the support is predetermined. In a particular embodiment, although the probes deposited on the solid support maintain a predetermined arrangement, they are not grouped by genetic variation but rather they have a random distribution, which, if desired, can always be the same.

In a particular embodiment, the support is a glass slide and, in this case, the probes, in the established number of replicas (6, 8 or 10), are printed in glass slides which are previously treated, for example, amino-silanized, using automatic DNA-chip production equipment by the deposition of the oligonucleotides in the glass slide (“microarrayer”) under suitable conditions, for example, by means of crosslinking with ultraviolet radiation and baking (80° C.), maintaining the humidity and temperature controlled during the deposition process, typically between 40-50% of relative humidity and 20° C. of temperature.

The replicas (probes) are distributed in the printing plates, containing the oligonucleotides in solution, such that they are printed by a number of different tips equal to half the replicas. The replicas are distributed homogeneously between the areas or sectors (sub-arrays) forming the DNA-chip. The number of replicas as well as their homogeneous distribution along the length and width of the DNA-chip minimize the experimental variability coming from the printing and hybridization processes. Likewise, positive and negative hybridization controls are printed. In general, each of the sub-arrays forming the DNA-chip is flanked by external hybridization controls which allow easily locating the points on the support. Although with the same sequence, the DNA-chip has two external hybridization controls labeled, for example, with a fluorophore (e.g., Cy3, Cy5, etc.), which serve to evaluate the hybridization quality in both channels. In a particular embodiment, the nucleotide sequence of the external control is the one previously identified as “CEH” and the sequences of the oligonucleotides for the detection thereof are those previously identified as ON1 and ON2.

A commercial DNA can be used to control the quality of the process for manufacturing the DNA-chip in terms of hybridization signal, background noise, specificity, sensitivity, reproducibility of each replica (coefficient of variation) as well as of the size and shape of the printed points (probes). By way of illustration, as a quality control of the printing of the DNA-chips of the invention, hybridization is carried out with a DNA with known genotype of one of every certain number of supports loaded with the probes, for example, every 20 printed supports. The correct genotyping of this control DNA is verified.

The inventors have designed, produced and validated the clinical use of the method of the invention in the detection of gene variants associated with pathologies associated with aging. Therefore, in a particular embodiment, the DNA-chip of the invention is a DNA-chip allowing the simultaneous, sensitive, specific and reproducible detection of gene variants associated with pathologies associated with aging; illustrative non-limiting examples of gene variants associated with aging which can be identified are shown in Table 1; nevertheless, the list of gene variants contained in said table can be increased with other gene variants which are gradually identified subsequently and which are associated with pathologies associated with aging. The sequences of all the genes mentioned in Table 1 are known and are shown, among others, on the following websites: GeneBank (NCBI), and Snpper.chip.org (Innate Immunity PGA).

In another aspect, the invention relates to a kit for putting the method of the invention into practice, hereinafter kit of the invention, comprising a DNA-chip of the invention comprising a support on which there is deposited a plurality of probes allowing the detection of human gene variants present in one or more genes associated with pathologies associated with aging. In a particular embodiment, the kit of the invention contains a protocol for the detection of said gene variants, comprising the use of an algorithm for the interpretation of the data generated with the application of said method; and, optionally, a protocol for the calculation of the risk conferred by said gene variants, comprising the use of various algorithms generated with the application of said method; and, optionally, a computer software facilitating, automatizing and assuring the reproducibility of the application of said algorithm for the interpretation of the data generated with the application of the invention.

The following example serves to illustrate the invention and must not be considered as limiting the scope thereof.

### Example 1

### Detection of Human Gene Variants (Polymorphisms) Associated with Pathologies Associated with Aging, Using a DNA-Chip

1.1 Design of the DNA-Chip

A DNA-chip was designed and manufactured to detect human gene variants, particularly SNPs (Single Nucleotide Polymorphisms) associated with pathologies associated with aging which allow the simultaneous, specific and reproducible detection of gene variants associated with said pathologies.

A list of gene variants associated with pathologies associated with aging is included below; nevertheless, probes which allow the identification of other gene variants associated with said diseases can be incorporated in the DNA-chip of the invention.

TABLE 1
Gene variants of pathologies associated with aging analyzed

| | |
|---|---|
| SNP01 | ACE intron 16 ins/del |
| SNP02 | ADRB1 Gly389Arg |
| SNP03 | ADRB2 Gln27Glu |
| SNP04 | ADRB2 Gly16Arg |
| SNP05 | ADRB3 Trp64Arg |
| SNP06 | AGT Met235Thr |
| SNP07 | AGTR1 1166 A > C |
| SNP08 | APOA1 −75 G > A |
| SNP09 | APOB Arg3480Trp |
| SNP10 | APOB Arg3500Gln |
| SNP11 | APOB Arg3531Cys |
| SNP12 | APOE Cys112Arg |
| SNP13 | APOE Arg158Cys |
| SNP14 | CBS 833 T > C |
| SNP15 | CBS 844ins68 |
| SNP16 | CETP TaqIB B1 > B2 |
| SNP17 | CETP Arg451Gln |
| SNP18 | COL1A1 1546 G > T |
| SNP19 | COMT Val158Met (Allele*2) |
| SNP20 | CYP17A1 −34 A > G |
| SNP21 | CYP19A1 1558 C > T |
| SNP22 | CYP1A1 Ile462Val |
| SNP23 | CYP1A1 T3801C |
| SNP24 | CYP1B1 Leu432Val |
| SNP25 | CYP1B1 Allele*4 (Asn453Ser) |
| SNP26 | CYP2C9 Arg144Cys (allele*2) |
| SNP27 | CYP2C9 Ile359Leu (allele*3) |
| SNP28 | CYP2C19 681 G > A (Pro227Pro) (allele*2) |
| SNP29 | CYP2D6 2549 A > del (allele*3) |
| SNP30 | CYP2D6 1847 G > A (allele*4) |
| SNP31 | CYP2D6 1707 del > T (allele*6) |
| SNP32 | ELAC2 Ala541Thr |
| SNP33 | ESR1 IVS1 −397 T > C (PvuII) p > P |
| SNP34 | F13A1 Val34Leu |
| SNP35 | FGB −455 G > A |
| SNP36 | FII 20210 G > A |
| SNP37 | FV Leiden Arg506Gln |
| SNP38 | GJA4 Pro319Ser |
| SNP39 | GNAS 393 T > C (Ile131Ile) |
| SNP40 | GNB3 825 C > T (Ser275Ser) |
| SNP41 | GSTM1 present > null |
| SNP42 | GSTP1 Ile105Val |
| SNP43 | GSTP1 Ala114Val |
| SNP44 | GSTT1 present > null |
| SNP45 | IL6 −174 C > G |
| SNP46 | IL10 −1082 G > A |
| SNP47 | ITGB3 Leu33Pro |
| SNP48 | MMP3 5A > 6A |
| SNP49 | MTHFR Ala222Val |
| SNP50 | NAT2 R64Q |
| SNP51 | NAT2 282 C > T (Y94Y) |
| SNP52 | NAT2 I114T |
| SNP53 | NAT2 481C > T (L161L) |
| SNP54 | NAT2 R197Q |
| SNP55 | NAT2 K268R |
| SNP56 | NAT2 G286E |
| SNP57 | NOS3 −786 T > C |
| SNP58 | NOS3 Glu298Asp |
| SNP59 | NPY Leu7Pro |
| SNP60 | OGG1 Cys326Ser |
| SNP61 | PAI1 4G > 5G |
| SNP62 | PGR 331 G > A |
| SNP63 | PON1 Gln192Arg |
| SNP64 | SOD2 Ala16Val |
| SNP65 | SRD5A2 Ala49Thr |
| SNP66 | SRD5A2 Val89Leu |
| SNP67 | SREBF2 Gly595Ala |
| SNP68 | SULT1A1 Arg213His |
| SNP69 | VDR b > B |

In this specific case, the designed and manufactured DNA-chip consists of a support (glass slide) containing on its surface a plurality of probes which allow the detection of the aforementioned gene variants. These probes are capable of hybridizing with the amplified target sequences of genes associated with pathologies associated with aging the genetic variation of which is to be analyzed. The DNA sequences of each of the probes used are the following [generally, the name of the gene and the genetic variation (change of the amino acid, change of nucleotide, “ins”: insertion, “del”: deletion) are indicated]:

Probes used

SNP01 ACE Intron 16 ins/del

| | |
|---|---|
| SEQ ID NO: 1 | GATTACAGGCGTGATACAGTCAC |
| SEQ ID NO: 2 | GTGACTGTATCACGCCTGTAATC |
| SEQ ID NO: 3 | AGACCTGCTGCCTATACAGTCAC |
| SEQ ID NO: 4 | GTGACTGTATAGGCAGCAGGTCT |

SNP02 ADRB1 Gly389Arg

| | |
|---|---|
| SEQ ID NO: 5 | AGGCCTTCCAGCGACTGCTCTGC |
| SEQ ID NO: 6 | GCAGAGCAGTCGCTGGAAGGCCT |
| SEQ ID NO: 7 | AGGCCTTCCAGGGACTGCTCTGC |
| SEQ ID NO: 8 | GCAGAGCAGTCCCTGGAAGGCCT |

SNP03 ADRB2 Gln27Glu

| | |
|---|---|
| SEQ ID NO: 9 | ACGTCACGCAGGAAAGGGACGAG |
| SEQ ID NO: 10 | CGTCACGCAGGAAAGGGACGA |
| SEQ ID NO: 11 | ACGTCACGCAGCAAAGGGACGAG |
| SEQ ID NO: 12 | CGTCACGCAGCAAAGGGACGA |

SNP04 ADRB2 Gly16Arg

| | |
|---|---|
| SEQ ID NO: 13 | TGGCACCCAATAGAAGCCATGCG |
| SEQ ID NO: 14 | CTGGCACCCAATAGAAGCCATGCGC |
| SEQ ID NO: 15 | TGGCACCCAATGGAAGCCATGCG |
| SEQ ID NO: 16 | CTGGCACCCAATGGAAGCCATGCGC |

SNP05 ADRB3 Trp64Arg

| | |
|---|---|
| SEQ ID NO: 17 | TGGCCATCGCCTGGACTCCGAGA |
| SEQ ID NO: 18 | TCTCGGAGTCCAGGCGATGGCCA |
| SEQ ID NO: 19 | TGGCCATCGCCCGGACTCCGAGA |
| SEQ ID NO: 20 | TCTCGGAGTCCGGGCGATGGCCA |

SNP06 AGT Met235Thr

| | |
|---|---|
| SEQ ID NO: 21 | GGCTGCTCCCTGACGGGAGCCAGTGTG |
| SEQ ID NO: 22 | CACACTGGCTCCCGTCAGGGAGCAGCC |
| SEQ ID NO: 23 | GGCTGCTCCCTGATGGGAGCCAGTGTG |
| SEQ ID NO: 24 | CACACTGGCTCCCATCAGGGAGCAGCC |

SNP07 AGTR1 1166 A > C

| | |
|---|---|
| SEQ ID NO: 25 | ACCAAATGAGCATTAGCTACTTT |
| SEQ ID NO: 26 | AAAGTAGCTAATGCTCATTTGGT |
| SEQ ID NO: 27 | ACCAAATGAGCCTTAGCTACTTT |
| SEQ ID NO: 28 | AAAGTAGCTAAGGCTCATTTGGT |

SNP08 APOA1 −75 G > A

| | |
|---|---|
| SEQ ID NO: 29 | AGCCCAGCCCCGGCCCTGTTG |
| SEQ ID NO: 30 | GCCCAGCCCCGGCCCTGTT |
| SEQ ID NO: 31 | AGCCCAGCCCTGGCCCTGTTG |
| SEQ ID NO: 32 | GCCCAGCCCTGGCCCTGTT |

SNP09 APOB Arg3480Trp

| | |
|---|---|
| SEQ ID NO: 33 | CGGTTCTTTCTCGGGAATATTCA |
| SEQ ID NO: 34 | TGAATATTCCCGAGAAAGAACCG |
| SEQ ID NO: 35 | CGGTTCTTTCTTGGGAATATTCA |
| SEQ ID NO: 36 | TGAATATTCCCAAGAAAGAACCG |

SNP10 APOB Arg3500Gln

| | |
|---|---|
| SEQ ID NO: 37 | CAAGAGCACACGGTCTTCAGTGA |
| SEQ ID NO: 38 | TCACTGAAGACCGTGTGCTCTTG |
| SEQ ID NO: 39 | CAAGAGCACACAGTCTTCAGTGA |
| SEQ ID NO: 40 | TCACTGAAGACTGTGTGCTCTTG |

SNP11 APOB Arg3531Cys

| | |
|---|---|
| SEQ ID NO: 41 | CCACACTCCAACGCATATATTCC |
| SEQ ID NO: 42 | GGAATATATGCGTTGGAGTGTGG |
| SEQ ID NO: 43 | CCACACTCCAATGCATATATTCC |
| SEQ ID NO: 44 | GGAATATATGCATTGGAGTGTGG |

SNP12 APOE Cys112Arg

| | |
|---|---|
| SEQ ID NO: 45 | ATGGAGGACGTGTGCGGCCGCCTGG |
| SEQ ID NO: 46 | CCAGGCGGCCGCACACGTCCTCCAT |
| SEQ ID NO: 47 | ATGGAGGACGTGCGCGGCCGCCTGG |
| SEQ ID NO: 48 | CCAGGCGGCCGCGCACGTCCTCCAT |

SNP13 APOE Arg158Cys

| | |
|---|---|
| SEQ ID NO: 49 | GACCTGCAGAAGCGCCTGGCAGTGT |
| SEQ ID NO: 50 | ACACTGCCAGGCGCTTCTGCAGGTC |
| SEQ ID NO: 51 | GACCTGCAGAAGTGCCTGGCAGTGT |
| SEQ ID NO: 52 | ACACTGCCAGGCACTTCTGCAGGTC |

SNP14 CBS 833 T > C

| | |
|---|---|
| SEQ ID NO: 53 | GATCCACCCCAGTGATCTGCAGA |
| SEQ ID NO: 54 | ATCCACCCCAGTGATCTGCAG |
| SEQ ID NO: 55 | GATCCACCCCAATGATCTGCAGA |
| SEQ ID NO: 56 | ATCCACCCCAATGATCTGCAG |

SNP15 CBS 844ins68

| | |
|---|---|
| SEQ ID NO: 57 | TGGGGTGGATCATCCAGGTGGGG |
| SEQ ID NO: 58 | CCCCACCTGGATGATCCACCCCA |
| SEQ ID NO: 59 | TGGGGTGGATCCCGAAGGGTCCA |
| SEQ ID NO: 60 | TGGACCCTTCGGGATCCACCCCA |

SNP16 CETP TaqIB B1 > B2

| | |
|---|---|
| SEQ ID NO: 61 | CACTGGGGTTCGAGTTAGGGTTC |
| SEQ ID NO: 62 | GAACCCTAACTCGAACCCCAGTG |
| SEQ ID NO: 63 | CACTGGGGTTCAAGTTAGGGTTC |
| SEQ ID NO: 64 | GAACCCTAACTTGAACCCCAGTG |

SNP17 CETP Arg451Gln

| | |
|---|---|
| SEQ ID NO: 65 | GATTATCACTCGAGATGTGAGTA |
| SEQ ID NO: 66 | ATTATCACTCGAGATGTGAGT |
| SEQ ID NO: 67 | GATTATCACTCAAGATGTGAGTA |
| SEQ ID NO: 68 | ATTATCACTCAAGATGTGAGT |

SNP18 COL1A1 1546 G > T

| | |
|---|---|
| SEQ ID NO: 69 | TCATCCCGCCCCCATTCCCTGGG |
| SEQ ID NO: 70 | CATCCCGCCCCCATTCCCTGG |
| SEQ ID NO: 71 | TCATCCCGCCCACATTCCCTGGG |
| SEQ ID NO: 72 | CATCCCGCCCACATTCCCTGG |

SNP19 COMT Val158Met (Allele*2)

| | |
|---|---|
| SEQ ID NO: 73 | ATTTCGCTGGCGTGAAGGACAAG |
| SEQ ID NO: 74 | CTTGTCCTTCACGCCAGCGAAAT |
| SEQ ID NO: 75 | ATTTCGCTGGCATGAAGGACAAG |
| SEQ ID NO: 76 | CTTGTCCTTCATGCCAGCGAAAT |

SNP20 CYP17A1 −34 A > G

| | |
|---|---|
| SEQ ID NO: 77 | TCTACTCCACTGCTGTCTATC |
| SEQ ID NO: 78 | AGATAGACAGCAGTGGAGTAGAA |
| SEQ ID NO: 79 | TCTACTCCACCGCTGTCTATC |
| SEQ ID NO: 80 | AGATAGACAGCGGTGGAGTAGAA |

SNP21 CYP19A1 1558 C > T

| | |
|---|---|
| SEQ ID NO: 81 | TGGTCAGTACCCACTCTGGAGCA |
| SEQ ID NO: 82 | TGCTCCAGAGTGGGTACTGACCA |
| SEQ ID NO: 83 | TGGTCAGTACCTACTCTGGAGCA |
| SEQ ID NO: 84 | TGCTCCAGAGTAGGTACTGACCA |

SNP22 CYP1A1 Ile462Val

| | |
|---|---|
| SEQ ID NO: 85 | TCGGTGAGACCATTGCCCGCTGG |
| SEQ ID NO: 86 | CCAGCGGGCAATGGTCTCACCGA |
| SEQ ID NO: 87 | TCGGTGAGACCGTTGCCCGCTGG |
| SEQ ID NO: 88 | CCAGCGGGCAACGGTCTCACCGA |

SNP23 CYP1A1 T3801C

| | |
|---|---|
| SEQ ID NO: 89 | TCCACCTCCTGGGCTCACA |
| SEQ ID NO: 90 | TCCACCTCCCGGGCTCACA |
| SEQ ID NO: 91 | TCCACCTCCTGGGCTCACA |
| SEQ ID NO: 92 | TCCACCTCCCGGGCTCACA |

SNP24 CYP1B1 Leu432Val

| | |
|---|---|
| SEQ ID NO: 93 | AATCATGACCCACTGAAGTGGCCTA |
| SEQ ID NO: 94 | TAGGCCACTTCAGTGGGTCATGATT |
| SEQ ID NO: 95 | AATCATGACCCAGTGAAGTGGCCTA |
| SEQ ID NO: 96 | TAGGCCACTTCACTGGGTCATGATT |

SNP25 CYP1B1 Allele*4 (Asn453Ser)

| | |
|---|---|
| SEQ ID NO: 97 | CGGCCTCATCAACAAGGACCTGA |
| SEQ ID NO: 98 | TCAGGTCCTTGTTGATGAGGCCG |
| SEQ ID NO: 99 | CGGCCTCATCAGCAAGGACCTGA |
| SEQ ID NO: 100 | TCAGGTCCTTGCTGATGAGGCCG |

SNP26 CYP2C9 Arg144Cys (allele*2)

| | |
|---|---|
| SEQ ID NO: 101 | GCATTGAGGACCGTGTTCAAGAG |
| SEQ ID NO: 102 | CTCTTGAACACGGTCCTCAATGC |
| SEQ ID NO: 103 | GCATTGAGGACTGTGTTCAAGAG |
| SEQ ID NO: 104 | CTCTTGAACACAGTCCTCAATGC |

SNP27 CYP2C9 Ile359Leu (allele*3)

| | |
|---|---|
| SEQ ID NO: 105 | TCCAGAGATACATTGACCTTCTC |
| SEQ ID NO: 106 | GAGAAGGTCAATGTATCTCTGGA |
| SEQ ID NO: 107 | TCCAGAGATACCTTGACCTTCTC |
| SEQ ID NO: 108 | GAGAAGGTCAAGGTATCTCTGGA |

SNP28 CYP2C19 681 G > A (Pro227Pro) (allele*2)

| | |
|---|---|
| SEQ ID NO: 109 | GATTATTTCCCGGGAACCCATAA |
| SEQ ID NO: 110 | ATTATTTCCCGGGAACCCATA |
| SEQ ID NO: 111 | GATTATTTCCCAGGAACCCATAA |
| SEQ ID NO: 112 | ATTATTTCCCAGGAACCCATA |

SNP29 CYP2D6 2549 A > del (allele*3)

| | |
|---|---|
| SEQ ID NO: 113 | CCAGGTCATCCTGTGCTCAGTTA |
| SEQ ID NO: 114 | CAGGTCATCCTGTGCTCAGTT |
| SEQ ID NO: 115 | CCAGGTCATCCGTGCTCAGTTAG |
| SEQ ID NO: 116 | CAGGTCATCCGTGCTCAGTTA |

SNP30 CYP2D6 1847 G > A (allele*4)

| | |
|---|---|
| SEQ ID NO: 117 | CCCACCCCCAGGACGCCCCTT |
| SEQ ID NO: 118 | CCACCCCCAGGACGCCCCT |
| SEQ ID NO: 119 | CCCACCCCCAAGACGCCCCTT |
| SEQ ID NO: 120 | CCACCCCCAAGACGCCCCT |

SNP31 CYP2D6 1707 del > T (allele*6)

| | |
|---|---|
| SEQ ID NO: 121 | GCTGGAGCAGTGGGTGACCGA |
| SEQ ID NO: 122 | CTGGAGCAGTGGGTGACCG |
| SEQ ID NO: 123 | CGCTGGAGCAGGGGTGACCGA |
| SEQ ID NO: 124 | GCTGGAGCAGGGGTGACCG |

SNP32 ELAC2 Ala541Thr

| | |
|---|---|
| SEQ ID NO: 125 | GCACCCTGGCTGCTGTGTTTGTG |
| SEQ ID NO: 126 | CACAAACACAGCAGCCAGGGTGC |
| SEQ ID NO: 127 | GCACCCTGGCTACTGTGTTTGTG |
| SEQ ID NO: 128 | CACAAACACAGTAGCCAGGGTGC |

SNP33 ESR1 IVS1 −397 T > C (PvuII) p > P

| | |
|---|---|
| SEQ ID NO: 129 | AATGTCCCAGCTGTTTTATGCTT |
| SEQ ID NO: 130 | ATGTCCCAGCTGTTTTATGCT |
| SEQ ID NO: 131 | AATGTCCCAGCCGTTTTATGCTT |
| SEQ ID NO: 132 | ATGTCCCAGCCGTTTTATGCT |

SNP34 F13A1 Val34Leu

| | |
|---|---|
| SEQ ID NO: 133 | AGCTTCAGGGCGTGGTGCCCCGG |
| SEQ ID NO: 134 | GCTTCAGGGCGTGGTGCCCCG |
| SEQ ID NO: 135 | AGCTTCAGGGCTTGGTGCCCCGG |
| SEQ ID NO: 136 | GCTTCAGGGCTTGGTGCCCCG |

SNP35 FGB −455 G > A

| | |
|---|---|
| SEQ ID NO: 137 | TTGATTTTAATGGCCCCTTTTGA |
| SEQ ID NO: 138 | TCAAAAGGGGCCATTAAAATCAA |
| SEQ ID NO: 139 | TTGATTTTAATAGCCCCTTTTGA |
| SEQ ID NO: 140 | TCAAAAGGGGCTATTAAAATCAA |

SNP36 FII 20210 G > A

| | |
|---|---|
| SEQ ID NO: 141 | TGACTCTCAGCGAGCCTCAATGC |
| SEQ ID NO: 142 | GCATTGAGGCTCGCTGAGAGTCA |
| SEQ ID NO: 143 | TGACTCTCAGCAAGCCTCAATGC |
| SEQ ID NO: 144 | GCATTGAGGCTTGCTGAGAGTCA |

SNP37 FV Leiden Arg506Gln

| | |
|---|---|
| SEQ ID NO: 145 | CCTGGACAGGCGAGGAATACAGG |
| SEQ ID NO: 146 | CCTGTATTCCTCGCCTGTCCAGG |
| SEQ ID NO: 147 | CCTGGACAGGCAAGGAATACAGG |
| SEQ ID NO: 148 | CCTGTATTCCTTGCCTGTCCAGG |

SNP38 GJA4 Pro319Ser

| | |
|---|---|
| SEQ ID NO: 149 | ATGGCCAAAAACCCCCAAGTCGT |
| SEQ ID NO: 150 | ACGACTTGGGGGTTTTTGGCCAT |
| SEQ ID NO: 151 | ATGGCCAAAAATCCCCAAGTCGT |
| SEQ ID NO: 152 | ACGACTTGGGGATTTTTGGCCAT |

SNP39 GNAS 393 T > C (Ile131Ile)

| | |
|---|---|
| SEQ ID NO: 153 | GTGGACTACATTCTGAGTGTGAT |
| SEQ ID NO: 154 | ATCACACTCAGAATGTAGTCCAC |
| SEQ ID NO: 155 | GTGGACTACATCCTGAGTGTGAT |
| SEQ ID NO: 156 | ATCACACTCAGGATGTAGTCCAC |

SNP40 GNB3 825 C > T (Ser275Ser)

| | |
|---|---|
| SEQ ID NO: 157 | GGCATCACGTCCGTGGCCTTCTC |
| SEQ ID NO: 158 | GAGAAGGCCACGGACGTGATGCC |
| SEQ ID NO: 159 | GGCATCACGTCTGTGGCCTTCTC |
| SEQ ID NO: 160 | GAGAAGGCCACAGACGTGATGCC |

SNP41 GSTM1 present > null

| | |
|---|---|
| SEQ ID NO: 161 | CACATATTCTTGGCCTTCTGCAGAT |
| SEQ ID NO: 162 | ATCTGCAGAAGGCCAAGAATATGTG |
| SEQ ID NO: 163 | CACATATTCTTGACCTTCTGCAGAT |
| SEQ ID NO: 164 | ATCTGCAGAAGGTCAAGAATATGTG |

SNP42 GSTP1 Ile105Val

| | |
|---|---|
| SEQ ID NO: 165 | GCTGCAAATACATCTCCCTCATC |
| SEQ ID NO: 166 | GATGAGGGAGATGTATTTGCAGC |
| SEQ ID NO: 167 | GCTGCAAATACGTCTCCCTCATC |
| SEQ ID NO: 168 | GATGAGGGAGACGTATTTGCAGC |

SNP43 GSTP1 Ala114Val

| | |
|---|---|
| SEQ ID NO: 169 | CTGGCAGGAGGCGGGCAAGGATG |
| SEQ ID NO: 170 | ATCCTTGCCCGCCTCCTGCCA |
| SEQ ID NO: 171 | CTGGCAGGAGGTGGGCAAGGATG |
| SEQ ID NO: 172 | ATCCTTGCCCACCTCCTGCCA |

SNP44 GSTT1 present > null

| | |
|---|---|
| SEQ ID NO: 173 | CTGCCTAGTGGGTTCACCTGCCCAC |
| SEQ ID NO: 174 | GTGGGCAGGTGAACCCACTAGGCAG |
| SEQ ID NO: 175 | CTGCCTAGTGGGGTCACCTGCCCAC |
| SEQ ID NO: 176 | GTGGGCAGGTGACCCCACTAGGCAG |

SNP45 IL6 −174 C > G

| | |
|---|---|
| SEQ ID NO: 177 | TTGTGTCTTGCGATGCTAAAGGA |
| SEQ ID NO: 178 | TCCTTTAGCATCGCAAGACACAA |
| SEQ ID NO: 179 | TTGTGTCTTGCCATGCTAAAGGA |
| SEQ ID NO: 180 | TCCTTTAGCATGGCAAGACACAA |

SNP46 IL10 −1082 G > A

| | |
|---|---|
| SEQ ID NO: 181 | CTTCTTTGGGAAGGGGAAGTAGG |
| SEQ ID NO: 182 | CCTACTTCCCCTTCCCAAAGAAG |
| SEQ ID NO: 183 | CTTCTTTGGGAGGGGGAAGTAGG |
| SEQ ID NO: 184 | CCTACTTCCCCCTCCCAAAGAAG |

SNP47 ITGB3 Leu33Pro

| | |
|---|---|
| SEQ ID NO: 185 | GCCCTGCCTCTGGGCTCACCT |
| SEQ ID NO: 186 | GAGGTGAGCCCAGAGGCAGGGCC |
| SEQ ID NO: 187 | GCCCTGCCTCCGGGCTCACCT |
| SEQ ID NO: 188 | GAGGTGAGCCCGGAGGCAGGGCC |

SNP48 MMP3 5A > 6A

| | |
|---|---|
| SEQ ID NO: 189 | ATGGGGGGAAAAAACCATGTCTT |
| SEQ ID NO: 190 | GGGGAAAAAACCATGTCTTGTC |
| SEQ ID NO: 191 | ATGGGGGGAAAAACCATGTCTTG |
| SEQ ID NO: 192 | GGGGAAAAACCATGTCTTGTCC |

SNP49 MTHFR Ala222Val

| | |
|---|---|
| SEQ ID NO: 193 | TCTGCGGGAGCCGATTTCATC |
| SEQ ID NO: 194 | TGATGAAATCGGCTCCCGCAGAC |
| SEQ ID NO: 195 | TCTGCGGGAGTCGATTTCATC |
| SEQ ID NO: 196 | TGATGAAATCGACTCCCGCAGAC |

SNP50 NAT2 R64Q

| | |
|---|---|
| SEQ ID NO: 197 | ACCACCCACCCCGGTTTCTTCTT |
| SEQ ID NO: 198 | CCACCCACCCCGGTTTCTTCT |
| SEQ ID NO: 199 | ACCACCCACCCTGGTTTCTTCTT |
| SEQ ID NO: 200 | CCACCCACCCTGGTTTCTTCT |

SNP51 NAT2 282 C > T (Y94Y)

| | |
|---|---|
| SEQ ID NO: 201 | AGGGTATTTTTACATCCCTCCAGTT |
| SEQ ID NO: 202 | GGGTATTTTTACATCCCTCCAGT |
| SEQ ID NO: 203 | AGGGTATTTTTATATCCCTCCAGTT |
| SEQ ID NO: 204 | GGGTATTTTTATATCCCTCCAGT |

SNP52 NAT2 I114T

| | |
|---|---|
| SEQ ID NO: 205 | GCAGGTGACCATTGACGGCAGGA |
| SEQ ID NO: 206 | CAGGTGACCATTGACGGCAGG |
| SEQ ID NO: 207 | GCAGGTGACCACTGACGGCAGGA |
| SEQ ID NO: 208 | CAGGTGACCACTGACGGCAGG |

SNP53 NAT2 481C > T (L161L)

| | |
|---|---|
| SEQ ID NO: 209 | GGAATCTGGTACCTGGACCAAATCA |
| SEQ ID NO: 210 | AGGAATCTGGTACCTGGACCAAATCAG |
| SEQ ID NO: 211 | GGAATCTGGTACTTGGACCAAATCA |
| SEQ ID NO: 212 | AGGAATCTGGTACTTGGACCAAATCAG |

SNP54 NAT2 R197Q

| | |
|---|---|
| SEQ ID NO: 213 | CGCTTGAACCTCGAACAATTGAAGA |
| SEQ ID NO: 214 | GCTTGAACCTCGAACAATTGAAG |
| SEQ ID NO: 215 | CGCTTGAACCTCAAACAATTGAAGA |
| SEQ ID NO: 216 | GCTTGAACCTCAAACAATTGAAG |

SNP55 NAT2 K268R

| | |
|---|---|
| SEQ ID NO: 217 | AAGAAGTGCTGAAAAATATATTTAA |
| SEQ ID NO: 218 | TTAAATATATTTTTCAGCACTTCTT |
| SEQ ID NO: 219 | AAGAAGTGCTGAGAAATATATTTAA |
| SEQ ID NO: 220 | TTAAATATATTTCTCAGCACTTCTT |

SNP56 NAT2 G286E

| | |
|---|---|
| SEQ ID NO: 221 | AACCTGGTGATGGATCCCTTACTAT |
| SEQ ID NO: 222 | ACCTGGTGATGGATCCCTTACTA |
| SEQ ID NO: 223 | AACCTGGTGATGAATCCCTTACTAT |
| SEQ ID NO: 224 | ACCTGGTGATGAATCCCTTACTA |

SNP57 NOS3 −786 T > C

| | |
|---|---|
| SEQ ID NO: 225 | TCTTCCCTGGCTGGCTGACCCTG |
| SEQ ID NO: 226 | CAGGGTCAGCCAGCCAGGGAAGA |
| SEQ ID NO: 227 | TCTTCCCTGGCCGGCTGACCCTG |
| SEQ ID NO: 228 | CAGGGTCAGCCGGCCAGGGAAGA |

SNP58 NOS3 Glu298Asp

| | |
|---|---|
| SEQ ID NO: 229 | GCCCCAGATGAGCCCCCAGAACT |
| SEQ ID NO: 230 | AGTTCTGGGGGCTCATCTGGGGC |
| SEQ ID NO: 231 | GCCCCAGATGATCCCCCAGAACT |
| SEQ ID NO: 232 | AGTTCTGGGGGATCATCTGGGGC |

SNP59 NPY Leu7Pro

| | |
|---|---|
| SEQ ID NO: 233 | CGGACAGCCCCAGTCGCTTGTTA |
| SEQ ID NO: 234 | TAACAAGCGACTGGGGCTGTCCG |
| SEQ ID NO: 235 | CGGACAGCCCCGGTCGCTTGTTA |
| SEQ ID NO: 236 | TAACAAGCGACCGGGGCTGTCCG |

SNP60 OGG1 Cys326Ser

| | |
|---|---|
| SEQ ID NO: 237 | CCTGCGCCAATCCCGCCATGCTC |
| SEQ ID NO: 238 | CTGCGCCAATCCCGCCATGCT |
| SEQ ID NO: 239 | CCTGCGCCAATGCCGCCATGCTC |
| SEQ ID NO: 240 | CTGCGCCAATGCCGCCATGCT |

SNP61 PAI1 4G > 5G

| | |
|---|---|
| SEQ ID NO: 241 | CTGACTCCCCCACGTGT |
| SEQ ID NO: 242 | CTGACTCCCCACGTGTC |
| SEQ ID NO: 243 | CTGACTCCCCCACGTGT |
| SEQ ID NO: 244 | CTGACTCCCCACGTGTC |

SNP62 PGR 331 G > A

| | |
|---|---|
| SEQ ID NO: 245 | CGGGAGATAAAAGAGCCGCGTGT |
| SEQ ID NO: 246 | ACACGCGGCTCTTTTATCTCCCG |
| SEQ ID NO: 247 | CGGGAGATAAAGGAGCCGCGTGT |
| SEQ ID NO: 248 | ACACGCGGCTCCTTTATCTCCCG |

SNP63 PON1 Gln192Arg

| | |
|---|---|
| SEQ ID NO: 249 | CCCCTACTTACAATCCTGGGAGA |
| SEQ ID NO: 250 | TCTCCCAGGATTGTAAGTAGGGG |
| SEQ ID NO: 251 | CCCCTACTTACGATCCTGGGAGA |
| SEQ ID NO: 252 | TCTCCCAGGATCGTAAGTAGGGG |

SNP64 SOD2 Ala16Val

| | |
|---|---|
| SEQ ID NO: 253 | GATACCCCAAAGCCGGAGCCAGC |
| SEQ ID NO: 254 | ATACCCCAAAGCCGGAGCCAG |
| SEQ ID NO: 255 | GATACCCCAAAACCGGAGCCAGC |
| SEQ ID NO: 256 | ATACCCCAAAACCGGAGCCAG |

SNP65 SRD5A2 Ala49Thr

| | |
|---|---|
| SEQ ID NO: 257 | CCCGCCTGCCAGCCCGCGCCGCC |
| SEQ ID NO: 258 | CCGCCTGCCAGCCCGCGCCGC |
| SEQ ID NO: 259 | CCCGCCTGCCAACCCGCGCCGCC |
| SEQ ID NO: 260 | CCGCCTGCCAACCCGCGCCGC |

SNP66 SRD5A2 Val89Leu

| | |
|---|---|
| SEQ ID NO: 261 | CCTCTTCTGCGTACATTACTT |
| SEQ ID NO: 262 | CTCTTCTGCGTACATTACT |
| SEQ ID NO: 263 | CCTCTTCTGCCTACATTACTT |
| SEQ ID NO: 264 | CTCTTCTGCCTACATTACT |

SNP67 SREBF2 Gly595Ala

| | |
|---|---|
| SEQ ID NO: 265 | GCTGCTGCCGGCAACCTACAA |
| SEQ ID NO: 266 | TTGTAGGTTGCCGGCAGCAGC |
| SEQ ID NO: 267 | GCTGCTGCCGCCAACCTACAA |
| SEQ ID NO: 268 | TTGTAGGTTGGCGGCAGCAGC |

SNP68 SULT1A1 Arg213His

| | |
|---|---|
| SEQ ID NO: 269 | TTTGTGGGGCGCTCCCTGCCA |
| SEQ ID NO: 270 | TTGTGGGGCGCTCCCTGCC |
| SEQ ID NO: 271 | TTTGTGGGGCACTCCCTGCCA |
| SEQ ID NO: 272 | TTGTGGGGCACTCCCTGCC |

SNP69 VDR b > B

| | |
|---|---|
| SEQ ID NO: 273 | GACAGGCCTGCGCATTCCCAATA |
| SEQ ID NO: 274 | TATTGGGAATGCGCAGGCCTGTC |
| SEQ ID NO: 275 | GACAGGCCTGCACATTCCCAATA |
| SEQ ID NO: 276 | TATTGGGAATGTGCAGGCCTGTC |

1.2 Production of the DNA-Chip for the Genotyping of Gene Variants Associated with Pathologies Associated with Aging: Printing and Processing of the Glass Slides

The probes capable of detecting the different previously identified gene variants are printed in the amino-silanized support (glass slide) using DMSO as a printing buffer. The printing is carried out with a spotter or oligonucleotide (probes) printer controlling the temperature and the relative humidity.

The binding of the probes to the support (glass slide) is carried out by means of crosslinking with ultraviolet radiation and baking as described in the documentation provided by the manufacturer (for example, Corning Lifesciences http://www.corning.com). The relative humidity during the deposition process is maintained between 40-50% and the temperature around 20° C.

1.3 Validation of the Clinical Usefulness of the DNA-Chip for the Identification of Gene Variants Associated with Pathologies Associated with Aging: Simultaneous, Sensitive, Specific and Reproducible Detection of Human Gene Variants Associated with Pathologies Associated with Aging

1.3.1 Preparation of the Sample to be Hybridized

DNA of the individual is extracted from a biological sample (for example, peripheral blood, saliva, etc) by means of a filtration protocol (for example, commercial kits by Macherey Nagel, Qiagen, etc).

All the exons and introns of interest are amplified by means of multiplex amplification using the suitable oligonucleotide primer pairs. Virtually any oligonucleotide primer pair can be used which allows the specific amplification of gene fragments in which the genetic variation to be detected exists, advantageously, those pairs which allow said amplification in the least possible number of amplification reactions; particularly oligonucleotide primers were selected which allow amplifying in only 5 multiplex amplification reactions the fragments necessary for the genotyping of the aforementioned 69 gene variants analyzed using the DNA-chip of the invention for the detection of gene variants associated with pathologies associated with aging.

The oligonucleotide primers used to carry out multiplex amplification for the detection of gene variants associated with pathologies associated with aging can be designed using the sequences of the corresponding genes as described in GenBank using, for example, the softwares:

Primer 3 (http://frodo.wi.mit.edu/cgi-bin/primer3/primer3 www.cgi) or

Web Primer (http://seq.yeastgenome.org/cgi-bin/web-primer)

The oligonucleotide primers used to amplify the corresponding gene variants associated with pathologies associated with aging by means of multiplex amplification are mentioned below.

### Oligonucleotide Primers Used

SNP01ACE Intron 16 ins/del

| | |
|---|---|
| SEQ ID NO: 277 | GGGACTCTGTAAGCCACTGC |
| SEQ ID NO: 278 | CCATGCCCATAACAGGTCTT |

SNP02 ADRB1 Gly389Arg

| | |
|---|---|
| SEQ ID NO: 279 | GGCCTTCAACCCCATCATCTA |
| SEQ ID NO: 280 | CCGGTCTCCGTGGGTCGCGT |

SNP03 ADRB2 Gln27Glu

| | |
|---|---|
| SEQ ID NO: 281 | GCTCACCTGCCAGACTGC |
| SEQ ID NO: 282 | GCCAGGACGATGAGAGACAT |

SNP04 ADRB2 Gly16Arg

| | |
|---|---|
| SEQ ID NO: 283 | GCTCACCTGCCAGACTGC |
| SEQ ID NO: 284 | GCCAGGACGATGAGAGACAT |

SNP05 ADRB3 Trp64Arg

| | |
|---|---|
| SEQ ID NO: 285 | CAATACCGCCAACACCAGT |
| SEQ ID NO: 286 | CGAAGTCACGAACACGTTG |

SNP06 AGT Met235Thr

| | |
|---|---|
| SEQ ID NO: 287 | GAACTGGATGTTGCTGCTGA |
| SEQ ID NO: 288 | TTGCCTTACCTTGGAAGTGG |

SNP07 AGTR1 1166 A > C

| | |
|---|---|
| SEQ ID NO: 289 | CCGCCCCTCAGATAATGTAA |
| SEQ ID NO: 290 | GCAAAATGTGGCTTTGCTTT |

SNP08 APOA1 −75 G > A

| | |
|---|---|
| SEQ ID NO: 291 | CACCTCCTTCTCGCAGTCTC |
| SEQ ID NO: 292 | GGGACAGAGCTGATCCTTGA |

SNP09 APOB Arg3480Trp

| | |
|---|---|
| SEQ ID NO: 293 | AGCCTCACCTCTTACTTTTCCATTGAGTC |
| SEQ ID NO: 294 | CGTTGGTGAAAAAGAGGCCCTCTA |

SNP10 APOB Arg3500Gln

| | |
|---|---|
| SEQ ID NO: 295 | AGCCTCACCTCTTACTTTTCCATTGAGTC |
| SEQ ID NO: 296 | CGTTGGTGAAAAAGAGGCCCTCTA |

SNP11 APOB Arg3531Cys

| | |
|---|---|
| SEQ ID NO: 297 | AGCCTCACCTCTTACTTTTCCATTGAGTC |
| SEQ ID NO: 298 | CGTTGGTGAAAAAGAGGCCCTCTA |

SNP12 APOE Cys112Arg

| | |
|---|---|
| SEQ ID NO: 299 | CTGTCCAAGGAGCTGCAG |
| SEQ ID NO: 300 | CTGTTCCACCAGGGGCCC |

SNP13 APOE Arg158Cys

| | |
|---|---|
| SEQ ID NO: 301 | CTGTCCAAGGAGCTGCAG |
| SEQ ID NO: 302 | CTGTTCCACCAGGGGCCC |

SNP14 CBS 833 T > C

| | |
|---|---|
| SEQ ID NO: 303 | GCTTTTGCTGGCCTTGAG |
| SEQ ID NO: 304 | GGGTGAGTTACAGGCTGCAC |

SNP15 CBS 844ins68

| | |
|---|---|
| SEQ ID NO: 305 | GCTTTTGCTGGCCTTGAG |
| SEQ ID NO: 306 | GGGTGAGTTACAGGCTGCAC |

SNP16 CETP TaqIB B1 > B2

| | |
|---|---|
| SEQ ID NO: 307 | GCAAACAGCCAGGTATAGGG |
| SEQ ID NO: 307 | AAGAGACTGAGGCCCAGAGA |

SNP17 CETP Arg451Gln

| | |
|---|---|
| SEQ ID NO: 309 | AGCCCTCATGAACAGCAAAG |
| SEQ ID NO: 310 | AATCCTGTCTGGGCCTCTCT |

SNP18 COL1A1 1546 G > T

| | |
|---|---|
| SEQ ID NO: 311 | AGCCGCTCCCATTCTCTTAG |
| SEQ ID NO: 312 | GCGTGGTAGAGACAGGAGGA |

SNP19 COMT Val158Met (Allele*2)

| | |
|---|---|
| SEQ ID NO: 313 | GGGCCTACTGTGGCTACTCA |
| SEQ ID NO: 314 | CCCTTTTTCCAGGTCTGACA |

SNP20 CYP17A1 −34 A > G

| | |
|---|---|
| SEQ ID NO: 315 | GGGCTCCAGGAGAATCTTTC |
| SEQ ID NO: 316 | AGGGTAAGCAGCAAGAGAGC |

SNP21 CYP19A1 1558 C > T

| | |
|---|---|
| SEQ ID NO: 317 | CCTTGCACCCAGATGAGACT |
| SEQ ID NO: 318 | GGCAAGGATGGATGATTTGT |

SNP22 CYP1A1 Ile462Val

| | |
|---|---|
| SEQ ID NO: 319 | TGATGGTGCTATCGACAAGG |
| SEQ ID NO: 320 | TTTGGAAGTGCTCACAGCAG |

SNP23 CYP1A1 T3801C

| | |
|---|---|
| SEQ ID NO: 321 | CCGCTGCACTTAAGCAGTCT |
| SEQ ID NO: 322 | GGCCCCAACTACTCAGAGG |

SNP24 CYP1B1 Leu432Val

| | |
|---|---|
| SEQ ID NO: 323 | ACCTCTGTCTTGGGCTACCA |
| SEQ ID NO: 324 | GCCAGGATGGAGATGAAGAG |

SNP25 CYP1B1 Allele*4 (Asn453Ser)

| | |
|---|---|
| SEQ ID NO: 325 | ACCTCTGTCTTGGGCTACCA |
| SEQ ID NO: 326 | GCCAGGATGGAGATGAAGAG |

SNP26 CYP2C9 Arg144Cys (allele*2)

| | |
|---|---|
| SEQ ID NO: 327 | CCTGGGATCTCCCTCCTAGT |
| SEQ ID NO: 328 | CCACCCTTGGTTTTTCTCAA |

SNP27 CYP2C9 Ile359Leu (allele*3)

| | |
|---|---|
| SEQ ID NO: 329 | CCACATGCCCTACACAGATG |
| SEQ ID NO: 330 | TCGAAAACATGGAGTTGCAG |

SNP28 CYP2C19 681 G > A (Pro227Pro) (allele*2)

| | |
|---|---|
| SEQ ID NO: 331 | CAACCAGAGCTTGGCATATTG |
| SEQ ID NO: 332 | TAAAGTCCCGAGGGTTGTTG |

SNP29 CYP2D6 2549 A > del (allele*3)

| | |
|---|---|
| SEQ ID NO: 333 | GGGCCTGAGACTTGTCCAGG |
| SEQ ID NO: 334 | GCCGAGAGCATACTCGGGAC |

SNP30 CYP2D6 1847 G > A (allele*4)

| | |
|---|---|
| SEQ ID NO: 335 | CCACGCGCACGTGCCCGTCCCA |
| SEQ ID NO: 336 | CCTGCAGAGACTCCTCGGTCTCTC |

SNP31 CYP2D6 1707 del > T (allele*6)

| | |
|---|---|
| SEQ ID NO: 337 | CCACGCGCACGTGCCCGTCCCA |
| SEQ ID NO: 338 | CCTGCAGAGACTCCTCGGTCTCTC |

SNP32 ELAC2 Ala541Thr

| | |
|---|---|
| SEQ ID NO: 339 | CCGACACGTCTCTGCTACTG |
| SEQ ID NO: 340 | AACAAAAGCTCTGGGCAAGT |

SNP33 ESR1 IVS1 −397 T > C (PvuII) p > P

| | |
|---|---|
| SEQ ID NO: 341 | AGGGTTATGTGGCAATGACG |
| SEQ ID NO: 342 | ACCAATGCTCATCCCAACTC |

SNP34 F13A1 Val34Leu

| | |
|---|---|
| SEQ ID NO: 343 | CATGCCTTTTCTGTTGTCTTCTT |
| SEQ ID NO: 344 | CCCAGTGGAGACAGAGGATG |

SNP35 FGB −455 G > A

| | |
|---|---|
| SEQ ID NO: 345 | GGGTCTTTCTGATGTGTATTTTTCA |
| SEQ ID NO: 346 | GACCTACTCACAAGGCAACCA |

SNP36 FII 20210 G > A

| | |
|---|---|
| SEQ ID NO: 347 | GAGAGTAGGGGGCCACTCAT |
| SEQ ID NO: 348 | CCTGAGCCCAGAGAGCTG |

SNP37 FV Leiden Arg506Gln

| | |
|---|---|
| SEQ ID NO: 349 | GCCCAGTGCTTAACAAGACC |
| SEQ ID NO: 350 | CCCATTATTTAGCCAGGAGACC |

SNP38 GJA4 Pro319Ser

| | |
|---|---|
| SEQ ID NO: 351 | CCTCCTCAGACCCTTACACG |
| SEQ ID NO: 352 | GCAGCCAGACTTCTCAGGAC |

SNP39 GNAS 393 T > C (Ile131Ile)

| | |
|---|---|
| SEQ ID NO: 353 | AGTACGTGCTGGCTCCTTGT |
| SEQ ID NO: 354 | CACAAGTCGGGGTGTAGCTT |

SNP40 GNB3 825 C > T (Ser275Ser)

| | |
|---|---|
| SEQ ID NO: 355 | CTGCCGCTTGTTTGACCT |
| SEQ ID NO: 356 | CACACGCTCAGACTTCATGG |

SNP41 GSTM1 present > null

| | |
|---|---|
| SEQ ID NO: 357 | TGCTTCACGTGTTATGGAGGT |
| SEQ ID NO: 358 | GGGCTCAAATATACGGTGGA |

SNP42 GSTP1 Ile105Val

| | |
|---|---|
| SEQ ID NO: 359 | CTCTATGGGAAGGACCAGCA |
| SEQ ID NO: 360 | GAAGCCCCTTTCTTTGTTCA |

SNP43 GSTP1 Ala114Val

| | |
|---|---|
| SEQ ID NO: 361 | GCAAGCAGAGGAGAATCTGG |
| SEQ ID NO: 362 | CTCACCTGGTCTCCCACAAT |

SNP44 GSTT1 present > null

| | |
|---|---|
| SEQ ID NO: 363 | GGCAGCATAAGCAGGACTTC |
| SEQ ID NO: 364 | CTGCAGTTGCTCGAGGACAA |

SNP45 IL6 −174 C > G

| | |
|---|---|
| SEQ ID NO: 365 | GCCTCAATGACGACCTAAGC |
| SEQ ID NO: 366 | TCATGGGAAAATCCCACATT |

SNP46 IL10 −1082 G > A

| | |
|---|---|
| SEQ ID NO: 367 | TCCCCAGGTAGAGCAACACT |
| SEQ ID NO: 368 | ATGGAGGCTGGATAGGAGGT |

SNP47 ITGB3 Leu33Pro

| | |
|---|---|
| SEQ ID NO: 369 | GCTCCAATGTACGGGGTAAA |
| SEQ ID NO: 370 | ACTCACTGGGAACTCGATGG |

SNP48 MMP3 5A > 6A

| | |
|---|---|
| SEQ ID NO: 371 | TCACTGCCACCACTCTGTTC |
| SEQ ID NO: 372 | GCCTCAACCTCTCAAAGTGC |

SNP49 MTHFR Ala222Val

| | |
|---|---|
| SEQ ID NO: 373 | GCCTCTCCTGACTGTCATCC |
| SEQ ID NO: 374 | CAAAGCGGAAGAATGTGTCA |

SNP50 NAT2 R64Q

| | |
|---|---|
| SEQ ID NO: 375 | CCATGGAGTTGGGCTTAGAG |
| SEQ ID NO: 376 | GGCTGATCCTTCCCAGAAAT |

SNP51 NAT2 282 C > T (Y94Y)

| | |
|---|---|
| SEQ ID NO: 377 | CCATGGAGTTGGGCTTAGAG |
| SEQ ID NO: 378 | CCATGCCAGTGCTGTATTTG |

SNP52 NAT2 I114T

| | |
|---|---|
| SEQ ID NO: 379 | CCATGGAGTTGGGCTTAGAG |
| SEQ ID NO: 380 | CCATGCCAGTGCTGTATTTG |

SNP53 NAT2 481C > T (L161L)

| | |
|---|---|
| SEQ ID NO: 381 | CAGGTGCCTTGCATTTTCT |
| SEQ ID NO: 382 | GATGAAGCCCACCAAACAGT |

SNP54 NAT2 R197Q

| | |
|---|---|
| SEQ ID NO: 383 | CAGGTGCCTTGCATTTTCT |
| SEQ ID NO: 384 | GATGAAGCCCACCAAACAGT |

SNP55 NAT2 K268R

| | |
|---|---|
| SEQ ID NO: 385 | AAAGACAATACAGATCTGGTCGAG |
| SEQ ID NO: 386 | TCTTCAAAATAACGTGAGGGTAGA |

SNP56 NAT2 G286E

| | |
|---|---|
| SEQ ID NO: 387 | AAAGACAATACAGATCTGGTCGAG |
| SEQ ID NO: 388 | TCTTCAAAATAACGTGAGGGTAGA |

SNP57 NOS3 −786 T > C

| | |
|---|---|
| SEQ ID NO: 389 | GTGTACCCCACCTGCATTCT |
| SEQ ID NO: 390 | CCCACCCTGTCATTCAGTG |

SNP58 NOS3 Glu298Asp

| | |
|---|---|
| SEQ ID NO: 391 | GAAGGCAGGAGACAGTGGAT |
| SEQ ID NO: 392 | CAGTCAATCCCTTTGGTGCT |

SNP59 NPY Leu7Pro

| | |
|---|---|
| SEQ ID NO: 393 | CTCTGCCTGGTGATGAGGTT |
| SEQ ID NO: 394 | GCAGAGGAGGGAGGTGCT |

SNP60 OGG1 Cys326Ser

| | |
|---|---|
| SEQ ID NO: 395 | TAGTCTCACCAGCCCTGACC |
| SEQ ID NO: 396 | TGGGGAATTTCTTTGTCCAG |

SNP61 PAI1 4G > 5G

| | |
|---|---|
| SEQ ID NO: 397 | CAACCTCAGCCAGACAAGGT |
| SEQ ID NO: 398 | CAGCCACGTGATTGTCTAGG |

SNP62 PGR 331 G > A

| | |
|---|---|
| SEQ ID NO: 399 | GCTTCACAGCATGCACGAGT |
| SEQ ID NO: 400 | GAGGACTGGAGACGCAGAGT |

SNP63 PON1 Gln192Arg

| | |
|---|---|
| SEQ ID NO: 401 | TATTGTTGCTGTGGGACCTG |
| SEQ ID NO: 402 | CAAATCCTTCTGCCACCACT |

SNP64 SOD2 Ala16Val

| | |
|---|---|
| SEQ ID NO: 403 | GGCTGTGCTTTCTCGTCTTC |
| SEQ ID NO: 404 | CCGTAGTCGTAGGGCAGGT |

SNP65 SRD5A2 Ala49Thr

| | |
|---|---|
| SEQ ID NO: 405 | AGCACACGGAGAGCCTGA |
| SEQ ID NO: 406 | AGGGGAAAAACGCTACCTGT |

SNP66 SRD5A2 Val89Leu

| | |
|---|---|
| SEQ ID NO: 407 | AGCACACGGAGAGCCTGA |
| SEQ ID NO: 408 | AGGGGAAAAACGCTACCTGT |

SNP67 SREBF2 Gly595Ala

| | |
|---|---|
| SEQ ID NO: 409 | GGCCAGTGACCATTAACACC |
| SEQ ID NO: 410 | TCTTCAAAGCCTGCCTCAGT |

SNP68 SULT1A1 Arg213His

| | |
|---|---|
| SEQ ID NO: 411 | GTAATCCGAGCCTCCACTGA |
| SEQ ID NO: 412 | GCTGTGGTCCATGAACTCCT |

SNP69 VDR b > B

| | |
|---|---|
| SEQ ID NO: 413 | CCTCACTGCCCTTAGCTCTG |
| SEQ ID NO: 414 | CCCGCAAGAAACCTCAAATA |

The multiplex amplifications are carried out simultaneously under the same time and temperature conditions which allow the specific amplification of the gene fragments in which the gene variant to be detected may exist. Once the multiplex amplification has ended, it is verified in agarose gel that an amplification reaction has taken place.

Then, the sample to be hybridized (amplification product) is subjected to fragmentation with a DNAse and the products resulting from the fragmentation process are subjected to an indirect labeling reaction. A terminal transferase incorporates a nucleotide bound to a specific binding molecule, for example, biotin, at the end of these small fragments.

Before applying the sample on the DNA-chip, the sample is denatured by means of heating at 95° C. for 5 minutes and the hybridization buffer, “ChipMap Kit Hybridization Buffer” (Ventana Medical System), is added.

1.3.2 Hybridization

Hybridization is carried out automatically in the Ventana Discovery hybridization station (Ventana Medical Systems).

Prehybridization or blocking of the slide with BSA is carried out. Then, the sample together with the hybridization solution [ChipMap Kit Hybridization Buffer (Ventana Medical System)] is applied and is maintained for 1 hour at 45° C. following the Ventana 9.0 Europe protocol (Ventana Medical System). Finally, the slide is subjected to the action of different washing solutions [ChipMap hybridization Kit Buffers (Ventana Medical System)]. Once the hybridization process has ended, the final washing and drying of the slide is performed.

After hybridization has ended, development with streptavidin-Cy3 marks the points (probes) in which hybridization has taken place.

1.3.3. Scanning of the Slide

The slide is introduced in the confocal fluorescence scanner, for example Axon 4100A scanner, and the signal emitted by the standard labeling upon being excited by a laser is scanned.

1.3.4 Quantification of the Image

The software of the scanner itself allows quantification in the image obtained of the signal of the points in which hybridization has occurred.

1.3.5 Interpretation of the Results

Determination of the genotype of the individual, with respect to the human gene variants associated with pathologies associated with aging.

The genotype of the individual is established from the signal which is obtained with the probes detecting the different gene variants. To that end, briefly, first the background noise of all the probes are subtracted from their absolute intensity values; then, the replicas corresponding to each of the 4 probes which are used to characterize each gene variant are grouped. The mean intensity value for each of the 4 probes is calculated using the bounded mean of the replicas to eliminate aberrant points. Once the mean intensity values for each of the probes are known, two ratios (ratio 1 and ratio 2) are calculated:

$$Ratio 1 = \frac{Mean intensity probe 1}{Mean intensity probe 1 + Mean intensity probe 2}$$

$$Ratio 2 = \frac{Mean intensity probe 3}{Mean intensity probe 3 + Mean intensity probe 4}$$

These ratios are substituted in three linear functions characterizing each of the three possible genotypes:

| | |
|---|---|
| AA | Function 1 |
| AB | Function 2 |
| BB | Function 3 |

The function having a higher absolute value determines the genotype that the patient has.

In this case, said linear functions are obtained by means of the analysis of 10 subjects for each of the three possible genotypes of the gene variant (AA, AB, BB). With the results, ratios 1 and 2 are calculated for the 30 subjects. These ratios serve as classification variables of the three groups to generate the linear functions. The classification capacity of the two probe pairs designed is evaluated with these three linear functions. In the event that the classification capacity is not 100%, the probes would be re-designed. New subjects characterized for each of the three genotypes form new ratios 1 and 2 in order to improve the linear combinations thereof which form the linear functions and, in summary, in order to improve the classification capacity of the algorithm based on these three functions.

Provided that ratios 1 and 2 are within the range of the ratios used to construct the groups, the mean fluorescence intensity of the 40 replicas with respect to the background noise is greater than 5 and the coefficient of variation of all the replicas of the DNA-chip is under 0.25 (using a confocal fluorescence scanner), the result of the linear functions is considered correct.

In summary, each mutation has in the slide 4 probes (repeated 10 times) for detection thereof. Two of said probes detect one gene variant and the other two detect the other gene variant.

In the case of a homozygous subject for gene variant A, said subject will not have gene variant B; accordingly, in the image obtained of the glass support the probes detecting gene variant B have a considerably inferior hybridization signal than that of gene variant A and vice versa; in this case, ratios 1 and 2 will tend to 1 and the subjects will be assigned as AA homozygotes.

In addition, a heterozygous subject for a certain gene variant has both gene variants; therefore, the probes that detect them have an equivalent hybridization signal. Ratios 1 and 2 will tend to 0.5 and the subjects will be assigned as AB heterozygotes.

1.3.6. Analysis of the Results:

The slide was introduced in the scanner and the signal emitted by the standard labeling upon being excited by a laser was scanned (section 1.3.3) and the image obtained from the signal of the points in which hybridization has occurred quantified (section 1.3.4).

The analysis of the results was conducted using the functions described in section 1.3.5. After genotyping the 69 human gene variants described in the Table 1, said variants are grouped by particular genetic risks.

Therefore, for the determination of a particular genetic risk, first the results obtained corresponding to each particular genetic risk are grouped together. Thus in this step, the gene variants corresponding to each particular genetic risk studied are grouped together. Tables 2, 4, 6, 8, 10, 13, 15, 17 and 25 show (see column 1) the gene variants associated with each of the particular pathologies associated with aging that are studied.

Subsequently, each genotype associated with each gene variant is standardized or scored. In this sense, said values will be comprised in a range of standardized values, in which the genotype or genotypes of the highest risk of suffering from a certain pathology will comprise the value of the upper limit of said range of values, and the genotype or genotypes of the lowest risk of suffering from a certain pathology will comprise the value of the lower limit of said range of values. Thus, according to the genotype present in the sample analyzed, a corresponding standardized value is assigned to said genotype.

The particular genetic risk is then calculated according to equation [1] or [2] depending on whether said particular genetic risk is formed (or not) by a combination of partial particular risks.

When the particular genetic risk is not formed by a combination of partial particular risks, said particular genetic risk is calculated by means of the equation [1]:

$$PGR = \frac{{\sum }_{i = 1}^{n} xi}{{\sum }_{i = 1}^{n} Lsi} \qquad [ 1 ]$$

where
   PGR represents the particular genetic risk to be calculated;
   xᵢ represents the standardized value of the genotype characterized for a gene variant in a sample, in relation to the particular genetic risk to be calculated;
   Lsᵢ represents the value of the upper limit of the range of standardized values assigned to each gene variant, in relation to the particular genetic risk to be calculated; and
   n is the number of gene variants analyzed in relation to the particular genetic risk to be calculated.

When the particular genetic risk is formed by a combination of partial particular risks, said particular genetic risk is calculated by means of equation [2]:

$$PGR = \frac{{\sum }_{i = 1}^{n} PPGRi}{no . PPGR} \qquad [ 2 ]$$

where
   PGR represents the particular genetic risk to be calculated;
   PPGRi represents the value calculated for each partial particular genetic risk which, in combination with other partial particular genetic risks, forms the particular genetic risk to be calculated, wherein said PPGRi is calculated by means of equation [3]:

$$PPGRi = \frac{{\sum }_{i = 1}^{n} xi}{{\sum }_{i = 1}^{n} Lsi} \qquad [ 3 ]$$

where
   PPGRi has the previously mentioned meaning;
   xᵢ represents the standardized value of the genotype characterized for a gene variant in a sample, in relation to the partial particular genetic risk to be calculated;
   Lsᵢ represents the value of the upper limit of the range of standardized values assigned to each gene variant, in relation to the partial particular genetic risk to be calculated; and
   n is the number of gene variants analyzed in relation to the partial particular genetic risk to be calculated; and
      no.PPGR is the number of partial particular genetic risks analyzed in relation to the partial particular genetic risk to be calculated.

Once the particular genetic risks are calculated, the global genetic risk is determined by means of equation [4]:

$$GGR = \frac{\sum PGR}{n} \qquad [ 4 ]$$

where
   GGR represents the global genetic risk to be calculated;
   PGR represents the value calculated for each particular genetic risk analyzed in relation to the global genetic risk to be calculated, and is calculated by means of the previously described equations [1] or [2]; and
   n is the number of particular genetic risks analyzed in relation to the global genetic risk to be calculated.

Merely by way of a non-limiting illustration in this example, the global genetic risk that the analyzed subject has of a pathology associated with aging comprises the determination of the following particular genetic risks:
1. Particular genetic risk associated with suffering from vascular disease (vascular risk) [Tables 2-12];
2. Particular genetic risk associated with osteoporosis (risk of osteoporosis) [Tables 13-14];
3. Particular genetic risk associated with carcinogenesis (carcinogenic risk) [Tables 15-16]; and
4. Particular genetic risk associated with environmental stress and oxidative damage [Tables 17-18].

Furthermore, the following partial particular genetic risks have been determined to determine the vascular risk:
partial particular genetic risk associated with lipid metabolism [Tables 2-3];
partial particular genetic risk associated with thrombosis [Tables 4-5];
partial particular genetic risk associated with ictus [Tables 6-7];
partial particular genetic risk associated with high blood pressure [Tables 8-9]; and
partial particular genetic risk associated with endothelial vulnerability [Tables 10-11].

Table 2 shows an example of how the value of a partial particular genetic risk, lipid metabolism, has been determined in a sample of a subject. In this case, the partial particular genetic risk associated with lipid metabolism has been calculated according to 11 SNPs (SNP08, SNP09, SNP10, SNP11, SNP12, SNP13, SNP17, SNP16, SNP63, SNP67 and SNP59).

Table 12 shows the result of the calculation of the vascular genetic risk, which has been calculated according to partial particular genetic risks: lipid metabolism, thrombosis, ictus, high blood pressure and endothelial vulnerability.

Table 24 shows the result of the calculation of the global genetic risk of suffering from a pathology associated with aging as explained above from an exemplary sample of a subject according to the particular genetic risks: vascular risk, osteoporosis risk, carcinogenic risk and environmental stress risk.

The particular genetic risk of the subject under study associated with response to drugs, i.e., the particular genetic risk of suffering from adverse reactions to drugs, has additionally been determined in this example. Table 25 shows the result of the general response to drugs in relation to those drugs metabolized by the following pathways: NAT2, CYP2D6, CYP2C19 and CYP2C9.

TABLE 2
VASCULAR RISK

| | | SCORE | | | | | |
|---|---|---|---|---|---|---|---|
| | | ACCORDING TO | | | | | |
| | LIPID METABOLISM | GENOTYPE | | | | | |
| SNP08 | APOA1 −75 G > A | G/G = 0 | G/A = 1 | A/A = 2 | | | |
| SNP09 | APOB Arg3480Trp | Arg/Arg = 0 | Arg/Trp = 1 | Trp/Trp = 2 | | | |
| SNP10 | APOB Arg3500Gln | Arg/Arg = 0 | Arg/Gln = 1 | Gln/Gln = 2 | | | |
| SNP11 | APOB Arg3531Cys | Arg/Arg = 0 | Arg/Cys = 1 | Cys/Cys = 2 | | | |
| SNP12-13* | APOE Alleles *2, *3, *4 Cys112Arg, Arg158Cys | E3/E3 = 0 | E3/E2 = 0 | E3/E4 = 1 | E2/E4 = 1 | E2/E2 = 2 | E4/E4 = 13.5 |
| SNP17 | CETP Arg451Gln | Arg/Arg = 0 | Arg/Gln = 1 | Gln/Gln = 2 | | | |
| SNP16 | CETP TaqlB B1/B2 | B2/B2 = 0 | B1/B2 = 1 | B1/B1 = 2 | | | |
| SNP63 | PON1 Gln192Arg | Gln/Gln = 0 | Gln/Arg = 0.5 | Arg/Arg = 1 | | | |
| SNP67 | SREBF2 Gly595Ala | Gly/Gly = 0 | Gly/Ala = 1 | Ala/Ala = 2 | | | |
| SNP59 | NPY Leu > Pro | Leu/Leu = 0 | Leu/Pro = 1 | Pro/Pro = 2 | | | |

*See Table 20

TABLE 3

| | Score of the |
|---|---|
| Genotype | Genotype |
| G/G | 0 |
| Arg/Arg | 0 |
| Arg/Arg | 0 |
| Arg/Arg | 0 |
| E3/E3 | 0 |
| Arg/Arg | 0 |
| B1/B2 | 1 |
| Gln/Gln | 0 |
| Gly/Ala | 1 |
| Leu/Leu | 0 |
| Sum | 2 |
| PPGR | 2/19 = 0.11 |

The summation of the maximum score of the upper limits of the ranges of values is 19 points (Σᵢ₌₁ⁿ Lsi = 19).
The risk is calculated on a scale of 0-1 considering the maximum value of 19 as risk = 1.

TABLE 4

| | | SCORE ACCORDING TO | | |
|---|---|---|---|---|
| | THROMBOSIS RISK | GENOTYPE | | |
| SNP61 | PAI1 4G > 5G | 5G/5G = 0 | 4G/5G = 1 | 4G/4G = 2 |
| SNP47 | ITGB3 Leu33Pro | Leu/Leu = 0 | Leu/Pro = 1 | Pro/Pro = 2 |
| SNP36 | FII 20210 G > A | G/G = 0 | G/A = 1 | A/A = 2 |
| SNP37 | FV Leiden Arg506Gln | Arg/Arg = 0 | Arg/Gln = 1 | Gln/Gln = 2 |
| SNP34 | F13A1 Val34Leu | Leu/Leu = 0 | Val/Leu = 1 | Val/Val = 2 |
| SNP49 | MTHFR Ala222Val | Ala/Ala = 0 | Ala/Val = 1 | Val/Val = 2 |
| SNP14 | CBS 833 T > C | T/T = 0 | T/C = 1 | C/C = 2 |
| SNP15 | CBS 844ins68 | Del/Del = 0 | Ins/Del = 1 | Ins/Ins = 2 |
| SNP35 | FGB −455 G > A | G/G = 0 men, =0 women | G/A = 1 men, =2 women | A/A = 2 men, =4 women |

TABLE 5

| | Score of the |
|---|---|
| Genotype | Genotype |
| 5G/4G | 1 |
| Leu/Pro | 1 |
| G/G | 0 |
| Arg/Arg | 0 |
| Val/Leu | 1 |
| Ala/Val | 1 |
| T/T | 0 |
| Del/Del | 0 |
| G/A | 1 |
| Sum | 5 |
| PPGR | 5/20 = 0.25 |

The summation of the maximum score of the upper limits of the ranges of values is 20 points (Σᵢ₌₁ⁿ Lsi = 20).
The risk is calculated on a scale of 0-1 considering the maximum value of 20 as risk = 1.

TABLE 6

| | | SCORE ACCORDING | | |
|---|---|---|---|---|
| | ICTUS RISK | TO GENOTYPE | | |
| SNP61 | PAI1 4G > 5G | 4G/4G = 0 | 4G/5G = 1 | 5G/5G = 2 |
| SNP47 | ITGB3 Leu33Pro | Leu/Leu = 0 | Leu/Pro = 1 | Pro/Pro = 2 |
| SNP36 | FII 20210 G > A | G/G = 0 | G/A = 1 | A/A = 2 |
| SNP37 | FV Leiden | Arg/Arg = 0 | Arg/Gln = 1 | Gln/Gln = 2 |
| | Arg506Gln | | | |
| SNP34 | F13A1 Val34Leu | Val/Val = 0 | Val/Leu = 1 | Leu/Leu = 2 |

TABLE 7

| | Score of the |
|---|---|
| Genotype | Genotype |
| 5G/4G | 1 |
| Leu/Pro | 1 |
| G/G | 0 |
| Arg/Arg | 0 |
| Val/Leu | 1 |
| Sum | 3 |
| PPGR | 3/10 = 0.30 |

The summation of the maximum score of the upper limits of the ranges of values is 30 points (Σᵢ₌₁ⁿ Lsi = 30).
The risk is calculated on a scale of 0-1 considering the maximum value of 30 as risk = 1

TABLE 8

| | HIGH BLOOD | SCORE ACCORDING TO | | |
|---|---|---|---|---|
| | PRESSURE RISK | GENOTYPE | | |
| SNP02 | ADRB1 Gly389Arg | Gly/Gly = 0 | Gly/Arg = 1 | Arg/Arg = 2 |
| SNP04 | ADRB2 Gly16Arg | Gly/Gly = 0 | Gly/Arg = 1 | Arg/Arg = 2 |
| SNP03 | ADRB2 Gln27Glu | Glu/Glu = 0 (1 if genotype ADRB2 | Gln/Glu = 1 (2 if genotype ADRB2 | Gln/Gln = 2 |
| | | Gly16Arg = Gly/Arg or Arg/Arg) | Gly16Arg = Gly/Arg or Arg/Arg) | |
| SNP06 | AGT Met235Thr | Met/Met = 0 | Met/Thr = 1 | Thr/Thr = 2 |
| SNP07 | AGTR1 1166 A > C | A/A = 0 | A/C = 1 | C/C = 2 |
| SNP39 | GNAS 393 T > C (Ile131Ile) | T/T = 2 | T/C = 1 | C/C = 0 |
| SNP40 | GNB3 825 C > T (Ser275Ser) | C/C = 0 | C/T = 1 | T/T = 2 |
| SNP01 | ACE Intron 16 ins/del | ins/ins = 0 | ins/del = 1 | del/del = 2 |
| SNP05 | ADRB3 Trp64Arg | Trp/Trp = 0 | Trp/Arg = 1 | Arg/Arg = 2 |

TABLE 9

| | Score of the |
|---|---|
| Genotype | Genotype |
| Arg/Arg | 2 |
| Gly/Arg | 1 |
| Gln/Gln | 2 |
| Thr/Thr | 2 |
| A/A | 0 |
| T/C | 1 |
| C/C | 0 |
| Del/Del | 2 |
| Trp/Arg | 1 |
| Sum | 11 |
| PPGR | 11/18 = 0.61 |

The summation of the maximum score of the upper limits of the ranges of values is 18 points (Σᵢ₌₁ⁿ Lsi = 18).
The risk is calculated on a scale of 0-1 considering the maximum value of 18 as risk = 1

TABLE 10

| | ENDOTHELIAL | SCORE ACCORDING TO | | |
|---|---|---|---|---|
| | VULNERABILITY RISK | GENOTYPE | | |
| SNP48 | MMP3 5A > 6A | 5A/5A = 1 | 5A/6A = 0 | 6A/6A = 1 |
| SNP57 | NOS3 −786 T > C | T/T = 0 | T/C = 1 (2 if genotype NOS3 | C/C = 2 (4 if genotype NOS3 Glu298Asp: |
| | | | Glu298Asp: Glu/Asp or Asp/Asp) | Glu/Asp or Asp/Asp) |
| SNP58 | NOS3 Glu298Asp | Glu/Glu = 0 | Glu/Asp = 1 | Asp/Asp = 2 |
| SNP49 | MTHFR Ala222Val | Ala/Ala = 0 | Ala/Val = 1 | Val/Val = 2 |
| SNP14 | CBS 833 T > C | T/T = 0 | T/C = 1 | C/C = 2 |
| SNP15 | CBS 844ins68 | del/del = 0 | ins/del = 1 | ins/ins = 2 |
| SNP38 | GJA4 Pro319Ser | Pro/Pro = 0 | Pro/Ser = 1 | Ser/Ser = 2 |

TABLE 11

| | Score of the |
|---|---|
| Genotype | Genotype |
| 6A/6A | 1 |
| T/T | 0 |
| Glu/Glu | 0 |
| Ala/Val | 1 |
| T/T | 0 |
| Del/Del | 0 |
| Pro/Ser | 1 |
| Sum | 3 |
| PPGR | 3/15 = 0.20 |

The summation of the maximum score of the upper limits of the ranges of values is 15 points (Σᵢ₌₁ⁿ Lsi = 15).
The risk is calculated on a scale of 0-1 considering the maximum value of 15 as risk = 1

TABLE 12
VASCULAR RISK

| | |
|---|---|
| LIPID METABOLISM | 0.11 |
| THROMBOSIS RISK | 0.25 |
| ICTUS RISK | 0.30 |
| HIGH BLOOD PRESSURE RISK | 0.61 |
| ENDOTHELIAL VULNERABILITY RISK | 0.20 |
| PGR: VASCULAR RISK | 0.29 |

TABLE 13

| | | SCORE ACCORDING | | |
|---|---|---|---|---|
| | OSTEOPOROSIS RISK | TO GENOTYPE | | |
| SNP18* | COL1A1 1546 G > T | G/G = 0 | G/T = 1 | T/T = 2 |
| SNP33 | ESR1 IVS1 −397 T > C p > P | p/p = 0 | p/P = 1 | P/P = 2 |
| | (Pvull) | | | |
| SNP69 | VDR b > B | b/b = 0 | b/B = 1 | B/B = 2 |

*only this polymorphism is considered in men

TABLE 14

| | Score of the |
|---|---|
| Genotype | Genotype |
| G/G | 0 |
| p/P | 1 |
| b/B | 1 |
| Sum | 2 |
| PGR | 2/6 = 0.33 |

The summation of the maximum score of the upper limits of the ranges of values is 6 points in women and 2 in men(Σᵢ₌₁ⁿ Lsi = 6 or 2).
The risk is calculated on a scale of 0-1 considering the maximum value of 6 or 2 as risk = 1

TABLE 15

| | | | SCORE ACCORDING | |
|---|---|---|---|---|
| | CARCINOGENIC RISK | | TO GENOTYPE | |
| SNP20* | CYP17A1 −34 A > G | A/A = 0 | A/G = 1 | G/G = 2 |
| SNP23* | CYP1A1 3801 T > C | T/T = 0 | T/C = 1 | C/C = 2 |
| SNP22* | CYP1A1 Ile462Val | Ile/Ile = 0 | Ile/Val = 1 | Val/Val = 2 |
| SNP24* | CYP1B1 Leu432Val | Val/Val = 2 | Val/Leu = 1 | Leu/Leu = 0 |
| SNP25* | CYP1B1 Allele*4 (Asn453Ser) | Asn/Asn = *1/*1 = 0 | Asn/Ser = *1/*4 = 1 | Ser/Ser = *4/*4 = 2 |
| SNP21* | CYP19A1 1558 C > T | C/C = 0 | C/T = 1 | T/T = 2 |
| SNP19** | COMT Val158Met (Allele*2) | Val/Val = 0 | Val/Met = 1 | Met/Met = 2 |
| SNP62** | PGR 331 G > A | G/G = 0 | G/A = 1 | A/A = 2 |
| SNP33** | ESR1 IVS1 −397 T > C p > P (Pvull) | p/p = 0 | p/P = 1 | P/P = 2 |
| SNP69** | VDR b > B | b/b = 0 | b/B = 1 | B/B = 2 |
| SNP65*** | SRD5A2 Ala49Thr | Ala/Ala = 0 | Ala/Thr = 1 | Thr/Thr = 2 |
| SNP66*** | SRD5A2 Val89Leu | Val/Val = 0 | Val/Leu = 1 | Leu/Leu = 2 |
| SNP32*** | ELAC2 Ala541Thr | Ala/Ala = 0 | Ala/Thr = 12.8 | Thr/Thr = 12.8 |

*SNPs included in men and in women.
**SNPs included in women.
***SNPs included in men.

TABLE 16

| | Score of the |
|---|---|
| Genotype | Genotype |
| A/G | 1 |
| T/T | 0 |
| Ile/Ile | 0 |
| Leu/Val | 1 |
| *1/1 | 0 |
| C/T | 1 |
| Val/Met | 1 |
| G/G | 0 |
| p/P | 1 |
| b/B | 1 |
| Ala/Ala | 0 |
| Val/Leu | 1 |
| Ala/Ala | 0 |
| Sum | 7 |
| PGR | 7/26 = 0.27 |

The summation of the maximum score of the upper limits of the ranges of values is 18 points in men and 20 in women (Σᵢ₌₁ⁿ Lsi = 18 or 20).
The risk is calculated on a scale of 0-1 considering the maximum value of 18 or 20 as risk = 1

TABLE 17
ENVIRONMENTAL STRESS RISK

| | ENVIRONMENTAL | | SCORE ACCORDING TO | |
|---|---|---|---|---|
| | STRESS RISK | | GENOTYPE | |
| SNP60 | OGG1 Cys326Ser | Cys/Cys = 2 | Cys/Ser = 1 | Ser/Ser = 0 |
| SNP64 | SOD2 Ala16Val | Ala/Ala = 0 | Ala/Val = 1 | Val/Val = 2 |
| SNP68 | SULT1A1 Arg213His | Arg/Arg = 0 | Arg/His = 1 | His/His = 2 |
| SNP41 | GSTM1 Present/Null | Present = 0 | Null = 1 | |
| SNP44 | GSTT1 Present/Null | Present = 0 | Null = 1 | |
| SNP42 | GSTP1 Ile105Val | Ile/Ile = 0 | Ile/Val = 1 (2 if genotype GSTM1 = | Val/Val = 2 (4 if genotype |
| | | | Null) | GSTM1 = Null) |
| SNP43 | GSTP1 Ala114Val | Ala/Ala = 0 | Ala/Val = 1 | Val/Val = 2 |
| SNP19 | COMT Val158Met (Allele*2) | Val/Val = 0 | Val/Met = 1 | Met/Met = 2 |
| SNP45 | IL6-174 C > G | C/C = 0 | C/G = 1 | G/G = 2 |
| SNP46 | IL10-1082 G > A | G/G = 0 | G/A = 1 | A/A = 2 |
| SNP50-51-52-53- | NAT2 Allele*4 (wt) | See Table 19 | | |
| 54-55-56 | | | | |

TABLE 18

| | Score of the |
|---|---|
| Genotype | Genotype |
| Cys/Ser | 1 |
| Ala/Val | 1 |
| Arg/Arg | 0 |
| Present | 0 |
| Present | 0 |
| Ile/Ile | 0 |
| Ala/Ala | 0 |
| Val/Met | 1 |
| C/G | 1 |
| G/G | 0 |
| *4/*5B or *5A/*12A | 1 |
| Sum | 5 |
| PGR | 5/22 = 0.23 |

The summation of the maximum score of the upper limits of the ranges of values is 22 points (Σᵢ₌₁ⁿ Lsi = 22).
The risk is calculated on a scale of 0-1 considering the maximum value of 22 as risk = 1

TABLE 19

| | SCORE | SNP50 | SNP51 | SNP52 | SNP53 | SNP54 | SNP55 | SNP56 | |
|---|---|---|---|---|---|---|---|---|---|
| | OF THE | NAT2 | NAT2 282 C > T | NAT2 | NAT2 481C > T | NAT2 | NAT2 | NAT2 | |
| Genotype | GENOTYPE | R64Q | (Y94Y) | I114T | (L161L) | R197Q | K268R | G286E | Metabolizer |
| *4/*4 | 0 | R/R | C/C | I/I | C/C | R/R | K/K | G/G | Fast |
| *4/*5A | 1 | R/R | C/C | I/T | C/T | R/R | K/K | G/G | Intermediate |
| *4/*5B or *5A/*12A | 1 | R/R | C/C | I/T | C/T | R/R | K/R | G/G | Intermediate |
| *4/*5C | 1 | R/R | C/C | I/T | C/C | R/R | K/R | G/G | Intermediate |
| *4/*6A | 1 | R/R | C/T | I/I | C/C | R/Q | K/K | G/G | Intermediate |
| *4/*6B | 1 | R/R | C/C | I/I | C/C | R/Q | K/K | G/G | Intermediate |
| *4/*7A | 1 | R/R | C/C | I/I | C/C | R/R | K/K | G/E | Intermediate |
| *4/*7B | 1 | R/R | C/T | I/I | C/C | R/R | K/K | G/E | Intermediate |
| *4/*12A | 0 | R/R | C/C | I/I | C/C | R/R | K/R | G/G | Fast |
| *4/*14A | 1 | R/Q | C/C | I/I | C/C | R/R | K/K | G/G | Intermediate |
| *4/*14B | 1 | R/Q | C/T | I/I | C/C | R/R | K/K | G/G | Intermediate |
| *5A/*5A | 2 | R/R | C/C | T/T | T/T | R/R | K/K | G/G | Slow |
| *5A/*5B | 2 | R/R | C/C | T/T | T/T | R/R | K/R | G/G | Slow |
| *5A/*5C | 2 | R/R | C/C | T/T | C/T | R/R | K/R | G/G | slow |
| *5A/*6A | 2 | R/R | C/T | I/T | C/T | R/Q | K/K | G/G | slow |
| *5A/*6B | 2 | R/R | C/C | I/T | C/T | R/Q | K/K | G/G | slow |
| *5A/*7A | 2 | R/R | C/C | I/T | C/T | R/R | K/K | G/E | slow |
| *5A/*7B | 2 | R/R | C/T | I/T | C/T | R/R | K/K | G/E | slow |
| *4/*5B or *5A/*12A | 1 | R/R | C/C | I/T | C/T | R/R | K/R | G/G | intermediate |
| *5A/*14A | 2 | R/Q | C/C | I/T | C/T | R/R | K/K | G/G | slow |
| *5A/*14B | 2 | R/Q | C/T | I/T | C/T | R/R | K/K | G/G | slow |
| *5B/*5B | 2 | R/R | C/C | T/T | T/T | R/R | R/R | G/G | slow |
| *5B/*5C | 2 | R/R | C/C | T/T | C/T | R/R | R/R | G/G | slow |
| *5B/*6A | 2 | R/R | C/T | I/T | C/T | R/Q | K/R | G/G | slow |
| *5B/*7A | 2 | R/R | C/C | I/T | C/T | R/R | K/R | G/E | slow |
| *5B/*7B | 2 | R/R | C/T | I/T | C/T | R/R | K/R | G/E | slow |
| *5B/*12A | 1 | R/R | C/C | I/T | C/T | R/R | R/R | G/G | intermediate |
| *5B/*14A | 2 | R/Q | C/C | I/T | C/T | R/R | K/R | G/G | slow |
| *5B/*14B | 2 | R/Q | C/T | I/T | C/T | R/R | K/R | G/G | slow |
| *5C/*5C | 2 | R/R | C/C | T/T | C/C | R/R | R/R | G/G | slow |
| *5C/*6A | 2 | R/R | C/T | I/T | C/C | R/Q | K/R | G/G | slow |
| *5C/*6B | 2 | R/R | C/C | I/T | C/C | R/Q | K/R | G/G | slow |
| *5C/*7A | 2 | R/R | C/C | I/T | C/C | R/R | K/R | G/E | slow |
| *5C/*7B | 2 | R/R | C/T | I/T | C/C | R/R | K/R | G/E | slow |
| *5C/*12A | 1 | R/R | C/C | I/T | C/C | R/R | R/R | G/G | intermediate |
| *5C/*14A | 2 | R/Q | C/C | I/T | C/C | R/R | K/R | G/G | slow |
| *5C/*14B | 2 | R/Q | C/T | I/T | C/C | R/R | K/R | G/G | slow |
| *6A/*6A | 2 | R/R | T/T | I/I | C/C | Q/Q | K/K | G/G | slow |
| *6A/*6B | 2 | R/R | C/T | I/I | C/C | Q/Q | K/K | G/G | slow |
| *6A/*7A or *6B/*7B | 2 | R/R | C/T | I/I | C/C | R/Q | K/K | G/E | slow |
| *6A/*7B | 2 | R/R | T/T | I/I | C/C | R/Q | K/K | G/E | slow |
| *6A/*12A | 1 | R/R | C/T | I/I | C/C | R/Q | K/R | G/G | intermediate |
| *6A/*14A or | 2 | R/Q | C/T | I/I | C/C | R/Q | K/K | G/G | slow |
| *6B/*14B | | | | | | | | | |
| *6A/*14B | 2 | R/Q | T/T | I/I | C/C | R/Q | K/K | G/G | slow |
| *6B/*6B | 2 | R/R | C/C | I/I | C/C | Q/Q | K/K | G/G | slow |
| *6B/*7A | 2 | R/R | C/C | I/I | C/C | R/Q | K/K | G/E | slow |
| *6A/*7A or *6B/*7B | 2 | R/R | C/T | I/I | C/C | R/Q | K/K | G/E | slow |
| *6B/*12A | 1 | R/R | C/C | I/I | C/C | R/Q | K/R | G/G | intermediate |
| *6B/*14A | 2 | R/Q | C/C | I/I | C/C | R/Q | K/K | G/G | slow |
| *6A/*14A or | 2 | R/Q | C/T | I/I | C/C | R/Q | K/K | G/G | slow |
| *6B/*14B | | | | | | | | | |
| *7A/*7A | 2 | R/R | C/C | I/I | C/C | R/R | K/K | E/E | slow |
| *7A/*7B | 2 | R/R | C/T | I/I | C/C | R/R | K/K | E/E | slow |
| *7A/*12A | 1 | R/R | C/C | I/I | C/C | R/R | K/R | G/E | intermediate |
| *7A/*14A | 2 | R/Q | C/C | I/I | C/C | R/R | K/K | G/E | slow |
| *7A/*14B or | 2 | R/Q | C/T | I/I | C/C | R/R | K/K | G/E | slow |
| *7B/*14A | | | | | | | | | |
| *7B/*7B | 2 | R/R | T/T | I/I | C/C | R/R | K/K | E/E | slow |
| *7B/*12A | 1 | R/R | C/T | I/I | C/C | R/R | K/R | G/E | intermediate |
| *7A/*14B or | 2 | R/Q | C/T | I/I | C/C | R/R | K/K | G/E | slow |
| *7B/*14A | | | | | | | | | |
| *7B/*14B | 2 | R/Q | T/T | I/I | C/C | R/R | K/K | G/E | slow |
| *12A/*12A | 0 | R/R | C/C | I/I | C/C | R/R | R/R | G/G | fast |
| *12A/*14A | 1 | R/Q | C/C | I/I | C/C | R/R | K/R | G/G | intermediate |
| *12A/*14B | 1 | R/Q | C/T | I/I | C/C | R/R | K/R | G/G | intermediate |
| *14A/*14A | 2 | Q/Q | C/C | I/I | C/C | R/R | K/K | G/G | slow |
| *14A/*14B | 2 | Q/Q | C/T | I/I | C/C | R/R | K/K | G/G | slow |
| *14B/*14B | 2 | Q/Q | T/T | I/I | C/C | R/R | K/K | G/G | slow |

TABLE 20

| | SNP12 | SNP13 | SCORE OF THE |
|---|---|---|---|
| | APOE Cys112Arg | APOE Arg158Cys | GENOTYPE |
| E3/E3 | Cys/Cys | Arg/Arg | 0 |
| E3/E2 | Cys/Cys | Arg/Cys | 0 |
| E3/E4 | Cys/Arg | Arg/Arg | 1 |
| E2/E4 | Cys/Arg | Arg/Cys | 1 |
| E2/E2 | Cys/Cys | Cys/Cys | 2 |
| E4/E4 | Arg/Arg | Arg/Arg | 13.5 |

TABLE 21

| | | CYP2D6 | | |
|---|---|---|---|---|
| | | SNP29 | SNP30 | SNP31 |
| Genotype | Metabolizer | CYP2D6 2549A > del | CYP2D6 1847G > A | CYP2D6 1707T > del |
| Not | Not determined | A/A | G/G | T/T |
| determined | | | | |
| Not | Not determined | A/del | G/G | T/T |
| determined | | | | |
| Not | Not determined | A/A | G/A | T/T |
| determined | | | | |
| Not | Not determined | A/A | G/G | T/of the |
| determined | | | | |
| *3/*3 | Slow | del/del | G/G | T/T |
| *3/*4 | Slow | A/del | G/A | T/T |
| *3/*6 | Slow | A/del | G/G | T/del |
| *4/*4 | Slow | A/A | A/A | T/T |
| *4/*6 | Slow | A/A | G/A | T/del |
| *6/*6 | Slow | A/A | G/G | del/del |

TABLE 22
CYP2C19

| | | SNP28 |
|---|---|---|
| Genotype | Metabolizer | CYP2C19 681G > A |
| *1/*1 | fast | G/G |
| *1/*2 | intermediate | G/A |
| *2/*2 | slow | A/A |
| *1/*3 | fast | G/G |
| *2/*3 | slow | G/A |
| *3/*3 | slow | G/G |

TABLE 23
CYP2C9

| | | | SNP27 |
|---|---|---|---|
| | | SNP26 | CYP2C9 42614 |
| Genotype | Metabolizer | CYP2C9 3608 C > T | A > C |
| *1/*1 | fast | C/C | A/A |
| *1/*2 | intermediate | C/T | A/A |
| *1/*3 | slow | C/C | A/C |
| *2/*2 | slow | T/T | A/A |
| *2/*3 | slow | C/T | A/C |
| *3/*3 | very slow | C/C | C/C |

TABLE 24

| | GLOBAL GENETIC |
|---|---|
| | RISK SUMMARY |
| VASCULAR RISK | 0.29 |
| OSTEOPOROSIS RISK | 0.33 |
| CARCINOGENIC RISK | 0.27 |
| ENVIRONMENTAL STRESS | 0.23 |
| GGR | 0.28 |

TABLE 25
RESPONSE TO DRUGS

| | GENOTYPE | METABOLIZER | |
|---|---|---|---|
| | NAT2 (Allele *4 (wt)) | | |
| SNP50-51-52- | *5B/*5B | Slow | See Table 19 |
| 53-54-55-56 | | | |
| | CYP2D6 (Alleles *3, *4 and *6) | | |
| SNP29-30-31 | *4/*4 | Slow | See Table 21 |
| | CYP2C19 (Alleles *1 (wt) | | |
| | and *2) | | |
| SNP28 | *1/*1 | Fast | See Table 22 |
| | CYP2C9 (Alleles *1 (wt), *2 | | |
| | and *3) | | |
| SNP26-27 | *1/*3 | Fast | See Table 23 |

1. An in vitro method for determining the global genetic risk of a subject to develop a pathology associated with aging from a combination of particular genetic risks comprising:
i) simultaneously genotyping multiple human gene variants present in one or more genes of a subject associated with a pathology associated with aging in a biological sample of said subject;
ii) determining each particular genetic risk; and
iii) determining said global genetic risk according to the value of each particular genetic risk obtained in step ii).

2. Method according to claim 1, wherein said step i) is performed by means of DNA-chip analysis and/or gene sequencing.

3. Method according to claim 1, wherein said step ii) comprises:
i) grouping the results obtained relating to each particular genetic risk of developing a pathology associated with aging;
ii) standardizing the value of each genotype of each gene variant analyzed;
iii) calculating each particular genetic risk such that:
iiia) when said particular genetic risk is not formed by a combination of partial particular risks, said particular genetic risk is calculated by means of equation [1]:
$$PGR = \frac{{\sum }_{i = 1}^{n} xi}{{\sum }_{i = 1}^{n} Lsi} \qquad [ 1 ]$$
where
PGR represents the particular genetic risk to be calculated;
xᵢ represents the standardized value of the genotype characterized for a gene variant in a sample, in relation to the particular genetic risk to be calculated;
Lsᵢ represents the value of the upper limit of the range of standardized values assigned to each gene variant, in relation to the particular genetic risk to be calculated; and
n is the number of gene variants analyzed in relation to the particular genetic risk to be calculated; or, alternatively,
iiib) when said particular genetic risk is formed by a combination of partial particular risks, said particular genetic risk is calculated by means of equation [2]:
$$PGR = \frac{{\sum }_{i = 1}^{n} PPGRi}{no . PPGR} \qquad [ 2 ]$$
where
PGR represents the particular genetic risk to be calculated;
PPGRi represents the value calculated for each partial particular genetic risk which, in combination with other partial particular genetic risks, forms the particular genetic risk to be calculated, wherein said PPGRi is calculated by means of equation [3]:
$$PPGRi = \frac{{\sum }_{i = 1}^{n} xi}{{\sum }_{i = 1}^{n} Lsi} \qquad [ 3 ]$$
where
PPGRi has the previously mentioned meaning;
xᵢ represents the standardized value of the genotype characterized for a gene variant in a sample, in relation to the partial particular genetic risk to be calculated;
Lsᵢ represents the value of the upper limit of the range of standardized values assigned to each gene variant, in relation to the partial particular genetic risk to be calculated; and
n is the number of gene variants analyzed in relation to the partial particular genetic risk to be calculated; and
no.PPGR is the number of partial particular genetic risks analyzed in relation to the partial particular genetic risk to be calculated.

4. Method according to claim 1, wherein the global genetic risk is calculated by means of equation [4]:
$$GGR = \frac{\sum PGR}{n} \qquad [ 4 ]$$
where
GGR represents the global genetic risk to be calculated;
PGR represents the value calculated for each particular genetic risk analyzed in relation to the global genetic risk to be calculated, and is calculated by means of the previously described equations [1] or [2]; and
n is the number of particular genetic risks analyzed in relation to the global genetic risk to be calculated.

5. Method according to claim 1, wherein said particular genetic risk is selected from the group consisting of particular genetic risk associated with suffering from vascular disease (vascular risk), particular genetic risk associated with osteoporosis, particular genetic risk associated with carcinogenesis, and particular genetic risk associated with environmental stress and oxidative damage.

6. Method according to claim 5, wherein said vascular risk is determined according to the partial particular genetic risks selected from the group formed by partial particular genetic risk associated with lipid metabolism, partial particular genetic risk associated with thrombosis, partial particular genetic risk associated with ictus, partial particular genetic risk associated with high blood pressure and partial particular genetic risk associated with endothelial vulnerability.

7. Method according to claim 6, wherein said partial particular genetic risk associated with lipid metabolism is determined according to the gene variants selected from the group formed by −75 G>A of the APOA1 gene, Arg3480Trp of the APOB gene, Arg3500Gln of the APOB gene, Arg3531Cys of the APOB gene, Cys112Arg of the APOE gene, Arg158Cys of the APOE gene, Arg451Gln of the CETP gene, TaqIB B1>B2 of the CETP gene, Gln192Arg of the PON1 gene, Gly595Ala of the SREBF2 gene, Leu7Pro of the NPY gene and combinations thereof.

8. Method according to claim 6, wherein said particular genetic risk associated with thrombosis is determined according to the gene variants selected from the group formed by 4G>5G of the PAI1 gene, Leu33Pro of the ITGB3 gene, 20210 G>A of the FII gene, Arg506Gln of the FV Leiden gene, Val34Leu of the F13A1 gene, Ala222Val of the MTHFR gene, 833 T>C of the CBS gene, 844ins68 of the CBS gene, −455 G>A of the FGB gene and combinations thereof.

9. Method according to claim 6, wherein said partial particular genetic risk associated with ictus is determined according to the gene variants selected from the group formed by 4G>5G of the PAI1 gene, Leu33Pro of the ITGB3 gene, 20210 G>A of the FII gene, Arg506Gln of the FV Leiden gene, Val34Leu of the F13A1 gene and combinations thereof.

10. Method according to claim 6, wherein said partial particular genetic risk associated with high blood pressure is determined according to the gene variants selected from the group formed by Gly389Arg of the ADRB1 gene; Gln27Glu of the ADRB2 gene, Gly16Arg of the ADRB2 gene, Met235Thr of the AGT gene, 1166 A>C of the AGTR1 gene, 393 T>C (Ile131Ile) of the GNAS gene, 825 C>T (Ser275Ser) of the GNB3 gene, intron 16 ins/del of the ACE gene, Trp64Arg of the ADRB3 gene and combinations thereof.

11. Method according to claim 6, wherein said partial particular genetic risk associated with endothelial vulnerability is determined according to the gene variants selected from the group formed by 5A>6A of the MMP3 gene, −786 T>C of the NOS3 gene, Glu298Asp of the NOS3 gene, Ala222Val of the MTHFR gene, 833 T>C of the CBS gene, 844ins68 of the CBS gene, Pro319Ser of the GJA4 gene and combinations thereof.

12. Method according to claim 5, wherein said particular genetic risk associated with osteoporosis is determined according to the gene variants selected from the group formed by 1546 G>T of the COL1A1 gene, IVS1-397 T>C p>P) (PvuII) of the ESR1 gene, b>B of the VDR gene and combinations thereof.

13. Method according to claim 5, wherein said particular genetic risk associated with carcinogenesis is determined according to the gene variants selected from the group formed by −34 A>G of the CYP17A1 gene, Ile462Val of the CYP1A1 gene, T3801C of the CYP1A1 gene, Leu432Val of the CYP1B1 gene, Allele*4 (Asn453Ser) of the CYP1B1 gene, 1558 C>T of the CYP19A1 gene, Val158Met (Allele*2) of the COMT gene, 331 G>A of the PGR gene, IVS1-397 T>C p>P) (PvuII) of the ESR1 gene, b>B of the VDR gene, Ala49Thr of the SRD5A2 gene, Val89Leu of the SRD5A2 gene, Ala541Thr of the ELAC2 gene and combinations thereof.

14. Method according to claim 5, wherein said particular genetic risk associated with environmental stress and oxidative damage is determined according to the gene variants selected from the group formed by Cys326Ser of the OGG1 gene, Ala16Val of the SOD2 gene, Arg213H is of the SULT1A1 gene, present>null GSTM1, present>null GSTT1, Ile105Val of the GSTP1 gene, Ala 114Val of the GSTP1 gene, Val158Met (Allele*2) of the COMT gene, −174 C>G of the IL6 gene, −1082 G>A of the IL10 gene, R64Q of the NAT2 gene, 282 C>T (Y94Y) of the NAT2 gene, I114T of the NAT2 gene, 481C>T (L161L) of the NAT2 gene, R197Q of the NAT2 gene, K268R of the NAT2 gene, G286E of the NAT2 gene and combinations thereof.

15. Method according to claim 1, further comprising determining the particular genetic risk associated with the response to drugs.

16. Method according to claim 15, wherein said particular genetic risk associated with the response to drugs is determined according to the gene variants selected from the group formed by R64Q, 282 C>T (Y94Y), I114T, 481C>T (L161L), R197Q, K268R and G286E of the NAT2 gene; Arg144Cys (allele*2) and Ile359Leu (allele*3) of the CYP2C9 gene; 681 G>A (Pro227Pro) (allele*2) of the CYP2C19 gene; 2549 A>del (allele*3), 1847 G>A (allele*4) and 1707 del>T (allele*6) of the CYP2D6 gene; and combinations thereof.

17. Method according to claim 1, wherein said gene variant to be genotyped is selected from the group formed by the intron 16 ins/del polymorphism of the ACE gene; the Gly389Arg polymorphism of the ADRB1 gene; the Gln27Glu and Gly16Arg polymorphisms of the ADRB2 gene; the Trp64Arg polymorphism of the ADRB3 gene; the Met235Thr polymorphism of the AGT gene; the 1166 A>C polymorphism of the AGTR1 gene; the −75 G>A polymorphism of the APOA1 gene; the Arg3480Trp, Arg3500Gln, and Arg3531Cys polymorphisms of the APOB gene; the Cys112Arg and Arg158Cys polymorphisms of the APOE gene; the 833 T>C and 844ins68 polymorphisms of the CBS gene; the TaqIB B1>B2 and Arg451Gln polymorphisms of the CETP gene; the 1546 G>T polymorphism of the COL1A1 gene; the Val158Met (Allele*2) polymorphism of the COMT gene; the −34 A>G polymorphism of the CYP17A1 gene; the 1558 C>T polymorphism of the CYP19A1 gene; the Ile462Val and T3801C polymorphism of the CYP1A1 gene; the Leu432Val and Allele*4 (Asn453Ser) polymorphism of the CYP1B1 gene; the Arg144Cys (allele*2) and Ile359Leu (allele*3) polymorphism of the CYP2C9 gene; the 681 G>A (Pro227Pro) (allele*2) polymorphism of the CYP2C19 gene; the 2549 A>del (allele*3), 1847 G>A (allele*4) and 1707 del>T (allele*6) polymorphism of the CYP2D6 gene; the Ala541Thr polymorphism of the ELAC2 gene; the IVS1-397 T>C p>P) (PvuII) polymorphism of the ESR1 gene; the Val34Leu polymorphism of the F13A1 gene; the −455 G>A polymorphism of the FGB gene; the 20210 G>A polymorphism of the FII gene; the Arg506Gln polymorphism of the FV Leiden gene; the Pro319Ser polymorphism of the GJA4 gene; the 393 T>C (Ile131Ile) polymorphism of the GNAS gene; the 825 C>T (Ser275Ser) polymorphism of the GNB3 gene; the present>null GSTM1 polymorphism; the Ile105Val and Ala 114Val polymorphisms of the GSTP1 gene; the present>null GSTT1 polymorphism; the −174 C>G polymorphism of the IL6 gene; the −1082 G>A polymorphism of the IL10 gene; the Leu33Pro polymorphism of the ITGB3 gene; the 5A>6A polymorphism of the MMP3 gene; the Ala222Val polymorphism of the MTHFR gene; the R64Q, 282 C>T (Y94Y), I114T, 481C>T (L161L), R197Q, K268R and G286E polymorphisms of the NAT2 gene; the −786 T>C and Glu298Asp polymorphisms of the NOS3 gene; the Leu7Pro polymorphism of the NPY gene; the Cys326Ser polymorphism of the OGG1 gene; the 4G>5G polymorphism of the PAI1 gene; the 331 G>A polymorphism of the PGR gene; the Gln192Arg polymorphism of the PON1 gene; the Ala16Val polymorphism of the SOD2 gene; the Ala49Thr and Val89Leu polymorphisms of the SRD5A2 gene; the Gly595Ala polymorphism of the SREBF2 gene; the Arg213H is polymorphism of the SULT1A1 gene; the b>B polymorphism of the VDR gene; and combinations thereof.

18. Method according to claim 17, further comprising genotyping one or more additional gene variants associated with pathologies associated with aging.

19. A DNA-chip comprising a support on which there is deposited a plurality of probes useful for detecting human gene variants present in one or more genes, wherein said probes are selected from the group formed by the probes identified as SEQ ID NO: 1-13, SEQ ID NO: 15, SEQ ID NO: 17-44, SEQ ID NO: 53-128, SEQ ID NO: 130, SEQ ID NO: 132-172, SEQ ID NO: 181-200, SEQ ID NO: 202, SEQ ID NO: 204, SEQ ID NO: 206, SEQ ID NO: 208, SEQ ID NO: 210, SEQ ID NO: 212, SEQ ID NO: 222 and SEQ ID NO: 224-276.

20. A kit comprising a DNA-chip according to claim 19.

21. An oligonucleotide primer selected from the oligonucleotide primers identified as SEQ ID NO: 277-278, SEQ ID NO: 285-319, SEQ ID NO: 321-326, SEQ ID NO: 333-340, SEQ ID NO: 343-356, SEQ ID NO: 359-362, SEQ ID NO: 364, SEQ ID NO: 367, SEQ ID NO: 369-371, SEQ ID NO: 374, SEQ ID NO: 377-381, SEQ ID NO: 383, SEQ ID NO: 385-402 and SEQ ID NO: 404-414.


SEQUENCE LISTING (417 sequences)

SEQ ID NO: 1 - DNA, 23 bp, Artificial
  probe 1 for detecting the Intron 16 ins/del polymorphism in the ACE gene
gattacaggcgtgatacagtcac

SEQ ID NO: 2 - DNA, 23 bp, Artificial
  probe 2 for detecting the Intron 16 ins/del polymorphism in the ACE gene
gtgactgtatcacgcctgtaatc

SEQ ID NO: 3 - DNA, 23 bp, Artificial
  probe 3 for detecting the Intron 16 ins/del polymorphism in the ACE gene
agacctgctgcctatacagtcac

SEQ ID NO: 4 - DNA, 23 bp, Artificial
  probe 4 for detecting the Intron 16 ins/del polymorphism in the ACE gene
gtgactgtataggcagcaggtct

SEQ ID NO: 5 - DNA, 23 bp, Artificial
  probe 1 for detecting the ADRB1 Gly389Arg polymorphism in the ADRB1 gene
aggccttccagcgactgctctgc

SEQ ID NO: 6 - DNA, 23 bp, Artificial
  probe 2 for detecting the ADRB1 Gly389Arg polymorphism in the ADRB1 gene
gcagagcagtcgctggaaggcct

SEQ ID NO: 7 - DNA, 23 bp, Artificial
  probe 3 for detecting the ADRB1 Gly389Arg polymorphism in the ADRB1 gene
aggccttccagggactgctctgc

SEQ ID NO: 8 - DNA, 23 bp, Artificial
  probe 8 for detecting the ADRB1 Gly389Arg polymorphism in the ADRB1 gene
gcagagcagtccctggaaggcct

SEQ ID NO: 9 - DNA, 23 bp, Artificial
  probe 1 for detecting the Gln27Glu polymorphism in the ADRB2 gene
acgtcacgcaggaaagggacgag

SEQ ID NO: 10 - DNA, 21 bp, Artificial
  probe 2 for detecting the Gln27Glu polymorphism in the ADRB2 gene
cgtcacgcaggaaagggacga

SEQ ID NO: 11 - DNA, 23 bp, Artificial
  probe 3 for detecting the Gln27Glu polymorphism in the ADRB2 gene
acgtcacgcagcaaagggacgag

SEQ ID NO: 12 - DNA, 21 bp, Artificial
  probe 4 for detecting the Gln27Glu polymorphism in the ADRB2 gene
cgtcacgcagcaaagggacga

SEQ ID NO: 13 - DNA, 23 bp, Artificial
  probe 1 for detecting the Gly16Arg polymorphism in the ADRB2 gene
tggcacccaatagaagccatgcg

SEQ ID NO: 14 - DNA, 25 bp, Artificial
  probe 2 for detecting the Gly16Arg polymorphism in the ADRB2 gene
ctggcacccaatagaagccatgcgc

SEQ ID NO: 15 - DNA, 23 bp, Artificial
  probe 3 for detecting the Gly16Arg polymorphism in the ADRB2 gene
tggcacccaatggaagccatgcg

SEQ ID NO: 16 - DNA, 25 bp, Artificial
  probe 4 for detecting the Gly16Arg polymorphism in the ADRB2 gene
ctggcacccaatggaagccatgcgc

SEQ ID NO: 17 - DNA, 23 bp, Artificial
  probe 1 for detecting the Trp64Arg polymorphism in the ADRB3 gene
tggccatcgcctggactccgaga

SEQ ID NO: 18 - DNA, 23 bp, Artificial
  probe 2 for detecting the Trp64Arg polymorphism in the ADRB3 gene
tctcggagtccaggcgatggcca

SEQ ID NO: 19 - DNA, 23 bp, Artificial
  probe 3 for detecting the Trp64Arg polymorphism in the ADRB3 gene
tggccatcgcccggactccgaga

SEQ ID NO: 20 - DNA, 23 bp, Artificial
  probe 4 for detecting the Trp64Arg polymorphism in the ADRB3 gene
tctcggagtccgggcgatggcca

SEQ ID NO: 21 - DNA, 25 bp, Artificial
  probe 1 for detecting the Met235Thr polymorphi sm in the AGT gene
gctgctccctgacgggagccagtgt

SEQ ID NO: 22 - DNA, 27 bp, Artificial
  probe 2 for detecting the Met235Thr polymorphi sm in the AGT gene
cacactggctcccgtcagggagcagcc

SEQ ID NO: 23 - DNA, 25 bp, Artificial
  probe 3 for detecting the Met235Thr polymorphi sm in the AGT gene
gctgctccctgatgggagccagtgt

SEQ ID NO: 24 - DNA, 27 bp, Artificial
  probe 4 for detecting the Met235Thr polymorphi sm in the AGT gene
cacactggctcccatcagggagcagcc

SEQ ID NO: 25 - DNA, 23 bp, Artificial
  probe 1 for detecting the 1166 A>C polymorphism in the AGTR1 gene
accaaatgagcattagctacttt

SEQ ID NO: 26 - DNA, 23 bp, Artificial
  probe 2 for detecting the 1166 A>C polymorphism in the AGTR1 gene
aaagtagctaatgctcatttggt

SEQ ID NO: 27 - DNA, 23 bp, Artificial
  probe 3 for detecting the 1166 A>C polymorphism in the AGTR1 gene
accaaatgagccttagctacttt

SEQ ID NO: 28 - DNA, 23 bp, Artificial
  probe 4 for detecting the 1166 A>C polymorphism in the AGTR1 gene
aaagtagctaaggctcatttggt

SEQ ID NO: 29 - DNA, 21 bp, Artificial
  probe 1 for detecting the -75 G>A polymorphism in the APOA1 gene
agcccagccccggccctgttg

SEQ ID NO: 30 - DNA, 19 bp, Artificial
  probe 2 for detecting the -75 G>A polymorphism in the APOA1 gene
gcccagccccggccctgtt

SEQ ID NO: 31 - DNA, 21 bp, Artificial
  probe 3 for detecting the -75 G>A polymorphism in the APOA1 gene
agcccagccctggccctgttg

SEQ ID NO: 32 - DNA, 19 bp, Artificial
  probe 4 for detecting the -75 G>A polymorphism in the APOA1 gene
gcccagccctggccctgtt

SEQ ID NO: 33 - DNA, 23 bp, Artificial
  probe 1 for detecting the Arg3480Trp polymorphism in the APOB gene
cggttctttctcgggaatattca

SEQ ID NO: 34 - DNA, 23 bp, Artificial
  probe 2 for detecting the Arg3480Trp polymorphism in the APOB gene
tgaatattcccgagaaagaaccg

SEQ ID NO: 35 - DNA, 23 bp, Artificial
  probe 3 for detecting the Arg3480Trp polymorphism in the APOB gene
cggttctttcttgggaatattca

SEQ ID NO: 36 - DNA, 23 bp, Artificial
  probe 4 for detecting the Arg3480Trp polymorphism in the APOB gene
tgaatattcccaagaaagaaccg

SEQ ID NO: 37 - DNA, 23 bp, Artificial
  probe 1 for detecting the Arg3500Gln polymorphism in the APOB gene
caagagcacacggtcttcagtga

SEQ ID NO: 38 - DNA, 23 bp, Artificial
  probe 2 for detecting the Arg3500Gln polymorphism in the APOB gene
tcactgaagaccgtgtgctcttg

SEQ ID NO: 39 - DNA, 23 bp, Artificial
  probe 3 for detecting the Arg3500Gln polymorphism in the APOB gene
caagagcacacagtcttcagtga

SEQ ID NO: 40 - DNA, 23 bp, Artificial
  probe 4 for detecting the Arg3500Gln polymorphism in the APOB gene
tcactgaagactgtgtgctcttg

SEQ ID NO: 41 - DNA, 23 bp, Artificial
  probe 1 for detecting the Arg3531Cys polymorphism in the APOB gene
ccacactccaacgcatatattcc

SEQ ID NO: 42 - DNA, 23 bp, Artificial
  probe 2 for detecting the Arg3531Cys polymorphism in the APOB gene
ggaatatatgcgttggagtgtgg

SEQ ID NO: 43 - DNA, 23 bp, Artificial
  probe 3 for detecting the Arg3531Cys polymorphism in the APOB gene
ccacactccaatgcatatattcc

SEQ ID NO: 44 - DNA, 23 bp, Artificial
  probe 4 for detecting the Arg3531Cys polymorphism in the APOB gene
ggaatatatgcattggagtgtgg

SEQ ID NO: 45 - DNA, 25 bp, Artificial
  probe 1 for detecting the Cys112Arg polymorphism in the APOE gene
atggaggacgtgtgcggccgcctgg

SEQ ID NO: 46 - DNA, 25 bp, Artificial
  probe 2 for detecting the Cys112Arg polymorphism in the APOE gene
ccaggcggccgcacacgtcctccat

SEQ ID NO: 47 - DNA, 25 bp, Artificial
  probe 3 for detecting the Cys112Arg polymorphism in the APOE gene
atggaggacgtgcgcggccgcctgg

SEQ ID NO: 48 - DNA, 25 bp, Artificial
  probe 4 for detecting the Cys112Arg polymorphism in the APOE gene
ccaggcggccgcgcacgtcctccat

SEQ ID NO: 49 - DNA, 25 bp, Artificial
  probe 1 for detecting the Arg158Cys polymorphism in the APOE gene
gacctgcagaagcgcctggcagtgt

SEQ ID NO: 50 - DNA, 25 bp, Artificial
  probe 2 for detecting the Arg158Cys polymorphism in the APOE gene
acactgccaggcgcttctgcaggtc

SEQ ID NO: 51 - DNA, 25 bp, Artificial
  probe 3 for detecting the Arg158Cys polymorphism in the APOE gene
gacctgcagaagtgcctggcagtgt

SEQ ID NO: 52 - DNA, 25 bp, Artificial
  probe 4 for detecting the Arg158Cys polymorphism in the APOE gene
acactgccaggcacttctgcaggtc

SEQ ID NO: 53 - DNA, 23 bp, Artificial
  probe 1 for detecting the 833 T>C polymorphism in the CBS gene
gatccaccccagtgatctgcaga

SEQ ID NO: 54 - DNA, 21 bp, Artificial
  probe 2 for detecting the 833 T>C polymorphism in the CBS gene
atccaccccagtgatctgcag

SEQ ID NO: 55 - DNA, 23 bp, Artificial
  probe 3 for detecting the 833 T>C polymorphism in the CBS gene
gatccaccccaatgatctgcaga

SEQ ID NO: 56 - DNA, 21 bp, Artificial
  probe 4 for detecting the 833 T>C polymorphism in the CBS gene
atccaccccaatgatctgcag

SEQ ID NO: 57 - DNA, 23 bp, Artificial
  probe 1 for detecting the 844ins68 polymorphism in the CBS gene
tggggtggatcatccaggtgggg

SEQ ID NO: 58 - DNA, 23 bp, Artificial
  probe 2 for detecting the 844ins68 polymorphism in the CBS gene
ccccacctggatgatccacccca

SEQ ID NO: 59 - DNA, 23 bp, Artificial
  probe 3 for detecting the 844ins68 polymorphism in the CBS gene
tggggtggatcccgaagggtcca

SEQ ID NO: 60 - DNA, 23 bp, Artificial
  probe 4 for detecting the 844ins68 polymorphism in the CBS gene
tggacccttcgggatccacccca

SEQ ID NO: 61 - DNA, 23 bp, Artificial
  probe 1 for detecting the TaqIB polymorphism in the CETP gene
cactggggttcgagttagggttc

SEQ ID NO: 62 - DNA, 23 bp, Artificial
  probe 2 for detecting the TaqIB polymorphism in the CETP gene
gaaccctaactcgaaccccagtg

SEQ ID NO: 63 - DNA, 23 bp, Artificial
  probe 3 for detecting the TaqIB polymorphism in the CETP gene
cactggggttcaagttagggttc

SEQ ID NO: 64 - DNA, 23 bp, Artificial
  probe 4 for detecting the TaqIB polymorphism in the CETP gene
gaaccctaacttgaaccccagtg

SEQ ID NO: 65 - DNA, 23 bp, Artificial
  probe 1 for detecting the Arg451Gln polymorphism in the CETP gene
gattatcactcgagatgtgagta

SEQ ID NO: 66 - DNA, 21 bp, Artificial
  probe 2 for detecting the Arg451Gln polymorphism in the CETP gene
attatcactcgagatgtgagt

SEQ ID NO: 67 - DNA, 23 bp, Artificial
  probe 3 for detecting the Arg451Gln polymorphism in the CETP gene
gattatcactcaagatgtgagta

SEQ ID NO: 68 - DNA, 21 bp, Artificial
  probe 4 for detecting the Arg451Gln polymorphism in the CETP gene
attatcactcaagatgtgagt

SEQ ID NO: 69 - DNA, 23 bp, Artificial
  probe 1 for detecting the 1546 G>T polymorphism in the COL1A1 gene
tcatcccgcccccattccctggg

SEQ ID NO: 70 - DNA, 21 bp, Artificial
  probe 2 for detecting the 1546 G>T polymorphism in the COL1A1 gene
catcccgcccccattccctgg

SEQ ID NO: 71 - DNA, 23 bp, Artificial
  probe 3 for detecting the 1546 G>T polymorphism in the COL1A1 gene
tcatcccgcccacattccctggg

SEQ ID NO: 72 - DNA, 21 bp, Artificial
  probe 4 for detecting the 1546 G>T polymorphism in the COL1A1 gene
catcccgcccacattccctgg

SEQ ID NO: 73 - DNA, 23 bp, Artificial
  probe 1 for detecting the Val158Met polymorphism (Allele*2) in the COMT gene
atttcgctggcgtgaaggacaag

SEQ ID NO: 74 - DNA, 23 bp, Artificial
  probe 2 for detecting the Val158Met polymorphism (Allele*2) in the COMT gene
cttgtccttcacgccagcgaaat

SEQ ID NO: 75 - DNA, 23 bp, Artificial
  probe 3 for detecting the Val158Met polymorphism (Allele*2) in the COMT gene
atttcgctggcatgaaggacaag

SEQ ID NO: 76 - DNA, 23 bp, Artificial
  probe 4 for detecting the Val158Met polymorphism (Allele*2) in the COMT gene
cttgtccttcatgccagcgaaat

SEQ ID NO: 77 - DNA, 21 bp, Artificial
  probe 1 for detecting the -34 A>G polymorphism in the CYP17A1 gene
tctactccactgctgtctatc

SEQ ID NO: 78 - DNA, 23 bp, Artificial
  probe 2 for detecting the -34 A>G polymorphism in the CYP17A1 gene
agatagacagcagtggagtagaa

SEQ ID NO: 79 - DNA, 21 bp, Artificial
  probe 3 for detecting the -34 A>G polymorphism in the CYP17A1 gene
tctactccaccgctgtctatc

SEQ ID NO: 80 - DNA, 23 bp, Artificial
  probe 4 for detecting the -34 A>G polymorphism in the CYP17A1 gene
agatagacagcggtggagtagaa

SEQ ID NO: 81 - DNA, 23 bp, Artificial
  probe 1 for detecting the 1558 C>T polymorphism in the CYP19A1 gene
tggtcagtacccactctggagca

SEQ ID NO: 82 - DNA, 23 bp, Artificial
  probe 2 for detecting the 1558 C>T polymorphism in the CYP19A1 gene
tgctccagagtgggtactgacca

SEQ ID NO: 83 - DNA, 23 bp, Artificial
  probe 3 for detecting the 1558 C>T polymorphism in the CYP19A1 gene
tggtcagtacctactctggagca

SEQ ID NO: 84 - DNA, 23 bp, Artificial
  probe 4 for detecting the 1558 C>T polymorphism in the CYP19A1 gene
tgctccagagtaggtactgacca

SEQ ID NO: 85 - DNA, 23 bp, Artificial
  probe 1 for detecting the Ile462Val polymorphism in the CYP1A1 gene
tcggtgagaccattgcccgctgg

SEQ ID NO: 86 - DNA, 23 bp, Artificial
  probe 2 for detecting the Ile462Val polymorphism in the CYP1A1 gene
ccagcgggcaatggtctcaccga

SEQ ID NO: 87 - DNA, 23 bp, Artificial
  probe 3 for detecting the Ile462Val polymorphism in the CYP1A1 gene
tcggtgagaccgttgcccgctgg

SEQ ID NO: 88 - DNA, 23 bp, Artificial
  probe 4 for detecting the Ile462Val polymorphism in the CYP1A1 gene
ccagcgggcaacggtctcaccga

SEQ ID NO: 89 - DNA, 19 bp, Artificial
  probe 1 for detecting the T3801C polymorphism in the CYP1A1 gene
tccacctcctgggctcaca

SEQ ID NO: 90 - DNA, 19 bp, Artificial
  probe 2 for detecting the T3801C polymorphism in the CYP1A1 gene
tccacctcccgggctcaca

SEQ ID NO: 91 - DNA, 19 bp, Artificial
  probe 3 for detecting the T3801C polymorphism in the CYP1A1 gene
tccacctcctgggctcaca

SEQ ID NO: 92 - DNA, 19 bp, Artificial
  probe 4 for detecting the T3801C polymorphism in the CYP1A1 gene
tccacctcccgggctcaca

SEQ ID NO: 93 - DNA, 25 bp, Artificial
  probe 1 for detecting the Leu432Val polymorphism in the CYP1B1 gene
aatcatgacccactgaagtggccta

SEQ ID NO: 94 - DNA, 25 bp, Artificial
  probe 2 for detecting the Leu432Val polymorphism in the CYP1B1 gene
taggccacttcagtgggtcatgatt

SEQ ID NO: 95 - DNA, 25 bp, Artificial
  probe 3 for detecting the Leu432Val polymorphism in the CYP1B1 gene
aatcatgacccagtgaagtggccta

SEQ ID NO: 96 - DNA, 25 bp, Artificial
  probe 4 for detecting the Leu432Val polymorphism in the CYP1B1 gene
taggccacttcactgggtcatgatt

SEQ ID NO: 97 - DNA, 23 bp, Artificial
  probe 1 for detecting the Allele*4 (Asn453Ser) polymorphism in the CYP1B1 gene
cggcctcatcaacaaggacctga

SEQ ID NO: 98 - DNA, 23 bp, Artificial
  probe 2 for detecting the Allele*4 (Asn453Ser) polymorphism in the CYP1B1 gene
tcaggtccttgttgatgaggccg

SEQ ID NO: 99 - DNA, 23 bp, Artificial
  probe 3 for detecting the Allele*4 (Asn453Ser) polymorphism in the CYP1B1 gene
cggcctcatcagcaaggacctga

SEQ ID NO: 100 - DNA, 23 bp, Artificial
  probe 4 for detecting the Allele*4 (Asn453Ser) polymorphism in the CYP1B1 gene
tcaggtccttgctgatgaggccg

SEQ ID NO: 101 - DNA, 23 bp, Artificial
  probe 1 for detecting the Arg144Cys (allele*2) polymorphism in the CYP2C9 gene
gcattgaggaccgtgttcaagag

SEQ ID NO: 102 - DNA, 23 bp, Artificial
  probe 2 for detecting the Arg144Cys (allele*2) polymorphism in the CYP2C9 gene
ctcttgaacacggtcctcaatgc

SEQ ID NO: 103 - DNA, 23 bp, Artificial
  probe 3 for detecting the Arg144Cys (allele*2) polymorphism in the CYP2C9 gene
gcattgaggactgtgttcaagag

SEQ ID NO: 104 - DNA, 23 bp, Artificial
  probe 4 for detecting the Arg144Cys (allele*2) polymorphism in the CYP2C9 gene
ctcttgaacacagtcctcaatgc

SEQ ID NO: 105 - DNA, 23 bp, Artificial
  probe 1 for detecting the Ile359Leu (allele*3) polymorphism in the CYP2C9 gene
tccagagatacattgaccttctc

SEQ ID NO: 106 - DNA, 23 bp, Artificial
  probe 2 for detecting the Ile359Leu (allele*3) polymorphism in the CYP2C9 gene
gagaaggtcaatgtatctctgga

SEQ ID NO: 107 - DNA, 23 bp, Artificial
  probe 3 for detecting the Ile359Leu (allele*3) polymorphism in the CYP2C9 gene
tccagagataccttgaccttctc

SEQ ID NO: 108 - DNA, 23 bp, Artificial
  probe 4 for detecting the Ile359Leu (allele*3) polymorphism in the CYP2C9 gene
gagaaggtcaaggtatctctgga

SEQ ID NO: 109 - DNA, 23 bp, Artificial
  probe 1 for detecting the 681 G>A (Pro227Pro) (allele*2) polymorphism in the CYP2C19 gene
gattatttcccgggaacccataa

SEQ ID NO: 110 - DNA, 21 bp, Artificial
  probe 2 for detecting the 681 G>A (Pro227Pro) (allele*2) polymorphism in the CYP2C19 gene
attatttcccgggaacccata

SEQ ID NO: 111 - DNA, 23 bp, Artificial
  probe 3 for detecting the 681 G>A (Pro227Pro) (allele*2) polymorphism in the CYP2C19 gene
gattatttcccaggaacccataa

SEQ ID NO: 112 - DNA, 21 bp, Artificial
  probe 4 for detecting the 681 G>A (Pro227Pro) (allele*2) polymorphism in the CYP2C19 gene
attatttcccaggaacccata

SEQ ID NO: 113 - DNA, 23 bp, Artificial
  probe 1 for detecting the 2549 A>del (allele*3) polymorphism in the CYP2D6 gene
ccaggtcatcctgtgctcagtta

SEQ ID NO: 114 - DNA, 21 bp, Artificial
  probe 2 for detecting the 2549 A>del (allele*3) polymorphism in the CYP2D6 gene
caggtcatcctgtgctcagtt

SEQ ID NO: 115 - DNA, 23 bp, Artificial
  probe 3 for detecting the 2549 A>del (allele*3) polymorphism in the CYP2D6 gene
ccaggtcatccgtgctcagttag

SEQ ID NO: 116 - DNA, 21 bp, Artificial
  probe 4 for detecting the 2549 A>del (allele*3) polymorphism in the CYP2D6 gene
caggtcatccgtgctcagtta

SEQ ID NO: 117 - DNA, 21 bp, Artificial
  probe 1 for detecting the 1846 G>A / 1847 G>A (allele*4) polymorphism in the CYP2D6 gene
cccacccccaggacgcccctt

SEQ ID NO: 118 - DNA, 19 bp, Artificial
  probe 2 for detecting the 1846 G>A / 1847 G>A (allele*4) polymorphism in the CYP2D6 gene
ccacccccaggacgcccct

SEQ ID NO: 119 - DNA, 21 bp, Artificial
  probe 3 for detecting the 1846 G>A / 1847 G>A (allele*4) polymorphism in the CYP2D6 gene
cccacccccaagacgcccctt

SEQ ID NO: 120 - DNA, 19 bp, Artificial
  probe 4 for detecting the 1846 G>A / 1847 G>A (allele*4) polymorphism in the CYP2D6 gene
ccacccccaagacgcccct

SEQ ID NO: 121 - DNA, 21 bp, Artificial
  probe 1 for detecting the 1707 del>T (allele*6) polymorphism in the CYP2D6 gene
gctggagcagtgggtgaccga

SEQ ID NO: 122 - DNA, 19 bp, Artificial
  probe 2 for detecting the 1707 del>T (allele*6) polymorphism in the CYP2D6 gene
ctggagcagtgggtgaccg

SEQ ID NO: 123 - DNA, 21 bp, Artificial
  probe 3 for detecting the 1707 del>T (allele*6) polymorphism in the CYP2D6 gene
cgctggagcaggggtgaccga

SEQ ID NO: 124 - DNA, 19 bp, Artificial
  probe 4 for detecting the 1707 del>T (allele*6) polymorphism in the CYP2D6 gene
gctggagcaggggtgaccg

SEQ ID NO: 125 - DNA, 23 bp, Artificial
  probe 1 for detecting the Ala541Thr polymorphism in the ELAC2 gene
gcaccctggctgctgtgtttgtg

SEQ ID NO: 126 - DNA, 23 bp, Artificial
  probe 2 for detecting the Ala541Thr polymorphism in the ELAC2 gene
cacaaacacagcagccagggtgc

SEQ ID NO: 127 - DNA, 23 bp, Artificial
  probe 3 for detecting the Ala541Thr polymorphism in the ELAC2 gene
gcaccctggctactgtgtttgtg

SEQ ID NO: 128 - DNA, 23 bp, Artificial
  probe 4 for detecting the Ala541Thr polymorphism in the ELAC2 gene
cacaaacacagtagccagggtgc

SEQ ID NO: 129 - DNA, 23 bp, Artificial
  probe 1 for detecting the -397 T>C (PvuII) polymorphism in the ESR1 IVS1 gene
aatgtcccagctgttttatgctt

SEQ ID NO: 130 - DNA, 21 bp, Artificial
  probe 2 for detecting the -397 T>C (PvuII) polymorphism in the ESR1 IVS1 gene
atgtcccagctgttttatgct

SEQ ID NO: 131 - DNA, 23 bp, Artificial
  probe 3 for detecting the -397 T>C (PvuII) polymorphism  in the ESR1 IVS1 gene
aatgtcccagccgttttatgctt

SEQ ID NO: 132 - DNA, 21 bp, Artificial
  probe 4 for detecting the -397 T>C (PvuII) polymorphism  in the ESR1 IVS1 gene
atgtcccagccgttttatgct

SEQ ID NO: 133 - DNA, 23 bp, Artificial
  probe 1 for detecting the Val34Leu polymorphism in the F13A1 gene
agcttcagggcgtggtgccccgg

SEQ ID NO: 134 - DNA, 21 bp, Artificial
  probe 2 for detecting the Val34Leu polymorphism in the F13A1 gene
gcttcagggcgtggtgccccg

SEQ ID NO: 135 - DNA, 23 bp, Artificial
  probe 3 for detecting the Val34Leu polymorphism in the F13A1 gene
agcttcagggcttggtgccccgg

SEQ ID NO: 136 - DNA, 21 bp, Artificial
  probe 4 for detecting the Val34Leu polymorphism in the F13A1 gene
gcttcagggcttggtgccccg

SEQ ID NO: 137 - DNA, 23 bp, Artificial
  probe 1 for detecting the -455 G>A polymorphism in the FGB gene
ttgattttaatggccccttttga

SEQ ID NO: 138 - DNA, 23 bp, Artificial
  probe 2 for detecting the -455 G>A polymorphism in the FGB gene
tcaaaaggggccattaaaatcaa

SEQ ID NO: 139 - DNA, 23 bp, Artificial
  probe 3 for detecting the -455 G>A polymorphism in the FGB gene
ttgattttaatagccccttttga

SEQ ID NO: 140 - DNA, 23 bp, Artificial
  probe 4 for detecting the -455 G>A polymorphism in the FGB gene
tcaaaaggggctattaaaatcaa

SEQ ID NO: 141 - DNA, 23 bp, Artificial
  probe 1 for detecting the 20210 G>A polymorphism in the FII gene
tgactctcagcgagcctcaatgc

SEQ ID NO: 142 - DNA, 23 bp, Artificial
  probe 2 for detecting the 20210 G>A polymorphism in the FII gene
gcattgaggctcgctgagagtca

SEQ ID NO: 143 - DNA, 23 bp, Artificial
  probe 3 for detecting the 20210 G>A polymorphism in the FII gene
tgactctcagcaagcctcaatgc

SEQ ID NO: 144 - DNA, 23 bp, Artificial
  probe 4 for detecting the 20210 G>A polymorphism in the FII gene
gcattgaggcttgctgagagtca

SEQ ID NO: 145 - DNA, 23 bp, Artificial
  probe 1 for detecting the Arg506Gln polymorphism in the FV Leiden gene
cctggacaggcgaggaatacagg

SEQ ID NO: 146 - DNA, 23 bp, Artificial
  probe 2 for detecting the Arg506Gln polymorphism in the FV Leiden gene
cctgtattcctcgcctgtccagg

SEQ ID NO: 147 - DNA, 23 bp, Artificial
  probe 3 for detecting the Arg506Gln polymorphism in the FV Leiden gene
cctggacaggcaaggaatacagg

SEQ ID NO: 148 - DNA, 23 bp, Artificial
  probe 4 for detecting the Arg506Gln polymorphism in the FV Leiden gene
cctgtattccttgcctgtccagg

SEQ ID NO: 149 - DNA, 23 bp, Artificial
  probe 1 for detecting the Pro319Ser polymorphism in the GJA4 gene
atggccaaaaacccccaagtcgt

SEQ ID NO: 150 - DNA, 23 bp, Artificial
  probe 2 for detecting the Pro319Ser polymorphism in the GJA4 gene
acgacttgggggtttttggccat

SEQ ID NO: 151 - DNA, 23 bp, Artificial
  probe 3 for detecting the Pro319Ser polymorphism in the GJA4 gene
atggccaaaaatccccaagtcgt

SEQ ID NO: 152 - DNA, 23 bp, Artificial
  probe 4 for detecting the Pro319Ser polymorphism in the GJA4 gene
acgacttggggatttttggccat

SEQ ID NO: 153 - DNA, 23 bp, Artificial
  probe 1 for detecting the 393 T>C (Ile131Ile) polymorphism in the GNAS gene
gtggactacattctgagtgtgat

SEQ ID NO: 154 - DNA, 23 bp, Artificial
  probe 2 for detecting the 393 T>C (Ile131Ile) polymorphism in the GNAS gene
atcacactcagaatgtagtccac

SEQ ID NO: 155 - DNA, 23 bp, Artificial
  probe 3 for detecting the 393 T>C (Ile131Ile) polymorphism in the GNAS gene
gtggactacatcctgagtgtgat

SEQ ID NO: 156 - DNA, 23 bp, Artificial
  probe 4 for detecting the 393 T>C (Ile131Ile) polymorphism in the GNAS gene
atcacactcaggatgtagtccac

SEQ ID NO: 157 - DNA, 23 bp, Artificial
  probe 1 for detecting the 825 C>T (Ser275Ser) polymorphism in the GNB3 gene
ggcatcacgtccgtggccttctc

SEQ ID NO: 158 - DNA, 23 bp, Artificial
  probe 2 for detecting the 825 C>T (Ser275Ser) polymorphism in the GNB3 gene
gagaaggccacggacgtgatgcc

SEQ ID NO: 159 - DNA, 23 bp, Artificial
  probe 3 for detecting the 825 C>T (Ser275Ser) polymorphism in the GNB3 gene
ggcatcacgtctgtggccttctc

SEQ ID NO: 160 - DNA, 23 bp, Artificial
  probe 4 for detecting the 825 C>T (Ser275Ser) polymorphism in the GNB3 gene
gagaaggccacagacgtgatgcc

SEQ ID NO: 161 - DNA, 25 bp, Artificial
  probe 1 for detecting the GSTM1 polymorphism
cacatattcttggccttctgcagat

SEQ ID NO: 162 - DNA, 25 bp, Artificial
  probe 2 for detecting the GSTM1 polymorphism
atctgcagaaggccaagaatatgtg

SEQ ID NO: 163 - DNA, 25 bp, Artificial
  probe 3 for detecting the GSTM1 polymorphism
cacatattcttgaccttctgcagat

SEQ ID NO: 164 - DNA, 25 bp, Artificial
  probe 4 for detecting the GSTM1 polymorphism
atctgcagaaggtcaagaatatgtg

SEQ ID NO: 165 - DNA, 23 bp, Artificial
  probe 1 for detecting the Ile105Val polymorphism in the GSTP1 gene
gctgcaaatacatctccctcatc

SEQ ID NO: 166 - DNA, 23 bp, Artificial
  probe 2 for detecting the Ile105Val polymorphism in the GSTP1 gene
gatgagggagatgtatttgcagc

SEQ ID NO: 167 - DNA, 23 bp, Artificial
  probe 3 for detecting the Ile105Val polymorphism in the GSTP1 gene
gctgcaaatacgtctccctcatc

SEQ ID NO: 168 - DNA, 23 bp, Artificial
  probe 4 for detecting the Ile105Val polymorphism in the GSTP1 gene
gatgagggagacgtatttgcagc

SEQ ID NO: 169 - DNA, 23 bp, Artificial
  probe 1 for detecting the Ala114Val polymorphism in the GSTP1 gene
ctggcaggaggcgggcaaggatg

SEQ ID NO: 170 - DNA, 21 bp, Artificial
  probe 2 for detecting the Ala114Val polymorphism in the GSTP1 gene
atccttgcccgcctcctgcca

SEQ ID NO: 171 - DNA, 23 bp, Artificial
  probe 3 for detecting the Ala114Val polymorphism in the GSTP1 gene
ctggcaggaggtgggcaaggatg

SEQ ID NO: 172 - DNA, 21 bp, Artificial
  probe 4 for detecting the Ala114Val polymorphism in the GSTP1 gene
atccttgcccacctcctgcca

SEQ ID NO: 173 - DNA, 25 bp, Artificial
  probe 1 for detecting the GSTT1 polymorphism
ctgcctagtgggttcacctgcccac

SEQ ID NO: 174 - DNA, 25 bp, Artificial
  probe 2 for detecting the GSTT1 polymorphism
gtgggcaggtgaacccactaggcag

SEQ ID NO: 175 - DNA, 25 bp, Artificial
  probe 3 for detecting the GSTT1 polymorphism
ctgcctagtggggtcacctgcccac

SEQ ID NO: 176 - DNA, 25 bp, Artificial
  probe 4 for detecting the GSTT1 polymorphism
gtgggcaggtgaccccactaggcag

SEQ ID NO: 177 - DNA, 23 bp, Artificial
  probe 1 for detecting the -174 C>G polymorphism in the IL6 gene
ttgtgtcttgcgatgctaaagga

SEQ ID NO: 178 - DNA, 23 bp, Artificial
  probe 2 for detecting the -174 C>G polymorphism in the IL6 gene
tcctttagcatcgcaagacacaa

SEQ ID NO: 179 - DNA, 23 bp, Artificial
  probe 3 for detecting the -174 C>G polymorphism in the IL6 gene
ttgtgtcttgccatgctaaagga

SEQ ID NO: 180 - DNA, 23 bp, Artificial
  probe 4 for detecting the -174 C>G polymorphism in the IL6 gene
tcctttagcatggcaagacacaa

SEQ ID NO: 181 - DNA, 23 bp, Artificial
  probe 1 for detecting the -1082 G>A polymorphism in the IL10 gene
cttctttgggaaggggaagtagg

SEQ ID NO: 182 - DNA, 23 bp, Artificial
  probe 2 for detecting the -1082 G>A polymorphism in the IL10 gene
cctacttccccttcccaaagaag

SEQ ID NO: 183 - DNA, 23 bp, Artificial
  probe 3 for detecting the -1082 G>A polymorphism in the IL10 gene
cttctttgggagggggaagtagg

SEQ ID NO: 184 - DNA, 23 bp, Artificial
  probe 4 for detecting the -1082 G>A polymorphism in the IL10 gene
cctacttccccctcccaaagaag

SEQ ID NO: 185 - DNA, 21 bp, Artificial
  probe 1 for detecting the Leu33Pro polymorphism in the ITGB3 gene
gccctgcctctgggctcacct

SEQ ID NO: 186 - DNA, 23 bp, Artificial
  probe 2 for detecting the Leu33Pro polymorphism in the ITGB3 gene
gaggtgagcccagaggcagggcc

SEQ ID NO: 187 - DNA, 21 bp, Artificial
  probe 3 for detecting the Leu33Pro polymorphism in the ITGB3 gene
gccctgcctccgggctcacct

SEQ ID NO: 188 - DNA, 23 bp, Artificial
  probe 4 for detecting the Leu33Pro polymorphism in the ITGB3 gene
gaggtgagcccggaggcagggcc

SEQ ID NO: 189 - DNA, 23 bp, Artificial
  probe 1 for detecting the 5A>6A polymorphism in the MMP3 gene
atggggggaaaaaaccatgtctt

SEQ ID NO: 190 - DNA, 22 bp, Artificial
  probe 2 for detecting the 5A>6A polymorphism in the MMP3 gene
ggggaaaaaaccatgtcttgtc

SEQ ID NO: 191 - DNA, 23 bp, Artificial
  probe 3 for detecting the 5A>6A polymorphism in the MMP3 gene
atggggggaaaaaccatgtcttg

SEQ ID NO: 192 - DNA, 22 bp, Artificial
  probe 4 for detecting the 5A>6A polymorphism in the MMP3 gene
ggggaaaaaccatgtcttgtcc

SEQ ID NO: 193 - DNA, 21 bp, Artificial
  probe 1 for detecting the Ala222Val polymorphism in the MTHFR gene
tctgcgggagccgatttcatc

SEQ ID NO: 194 - DNA, 23 bp, Artificial
  probe 2 for detecting the Ala222Val polymorphism in the MTHFR gene
tgatgaaatcggctcccgcagac

SEQ ID NO: 195 - DNA, 21 bp, Artificial
  probe 3 for detecting the Ala222Val polymorphism in the MTHFR gene
tctgcgggagtcgatttcatc

SEQ ID NO: 196 - DNA, 23 bp, Artificial
  probe 4 for detecting the Ala222Val polymorphism in the MTHFR gene
tgatgaaatcgactcccgcagac

SEQ ID NO: 197 - DNA, 23 bp, Artificial
  probe 1 for detecting the R64Q polymorphism in the NAT2 gene
accacccaccccggtttcttctt

SEQ ID NO: 198 - DNA, 21 bp, Artificial
  probe 2 for detecting the R64Q polymorphism in the NAT2 gene
ccacccaccccggtttcttct

SEQ ID NO: 199 - DNA, 23 bp, Artificial
  probe 3 for detecting the R64Q polymorphism in the NAT2 gene
accacccaccctggtttcttctt

SEQ ID NO: 200 - DNA, 21 bp, Artificial
  probe 4 for detecting the R64Q polymorphism in the NAT2 gene
ccacccaccctggtttcttct

SEQ ID NO: 201 - DNA, 25 bp, Artificial
  probe 1 for detecting the 282 C>T (Y94Y) polymorphism in the NAT2 gene
agggtatttttacatccctccagtt

SEQ ID NO: 202 - DNA, 23 bp, Artificial
  probe 2 for detecting the 282 C>T (Y94Y) polymorphism in the NAT2 gene
gggtatttttacatccctccagt

SEQ ID NO: 203 - DNA, 25 bp, Artificial
  probe 3 for detecting the 282 C>T (Y94Y) polymorphism in the NAT2 gene
agggtatttttatatccctccagtt

SEQ ID NO: 204 - DNA, 23 bp, Artificial
  probe 4 for detecting the 282 C>T (Y94Y) polymorphism in the NAT2 gene
gggtatttttatatccctccagt

SEQ ID NO: 205 - DNA, 23 bp, Artificial
  probe 1 for detecting the I114T polymorphism in the NAT2 gene
gcaggtgaccattgacggcagga

SEQ ID NO: 206 - DNA, 21 bp, Artificial
  probe 2 for detecting the I114T polymorphism in the NAT2 gene
caggtgaccattgacggcagg

SEQ ID NO: 207 - DNA, 23 bp, Artificial
  probe 3 for detecting the I114T polymorphism in the NAT2 gene
gcaggtgaccactgacggcagga

SEQ ID NO: 208 - DNA, 21 bp, Artificial
  probe 4 for detecting the I114T polymorphism in the NAT2 gene
caggtgaccactgacggcagg

SEQ ID NO: 209 - DNA, 25 bp, Artificial
  probe 1 for detecting the 481C>T (L161L) polymorphism in the NAT2 gene
ggaatctggtacctggaccaaatca

SEQ ID NO: 210 - DNA, 27 bp, Artificial
  probe 2 for detecting the 481C>T (L161L) polymorphism in the NAT2 gene
aggaatctggtacctggaccaaatcag

SEQ ID NO: 211 - DNA, 25 bp, Artificial
  probe 3 for detecting the 481C>T (L161L) polymorphism in the NAT2 gene
ggaatctggtacttggaccaaatca

SEQ ID NO: 212 - DNA, 27 bp, Artificial
  probe 4 for detecting the 481C>T (L161L) polymorphism in the NAT2 gene
aggaatctggtacttggaccaaatcag

SEQ ID NO: 213 - DNA, 25 bp, Artificial
  probe 1 for detecting the R197Q polymorphism in the NAT2 gene
cgcttgaacctcgaacaattgaaga

SEQ ID NO: 214 - DNA, 23 bp, Artificial
  probe 2 for detecting the R197Q polymorphism in the NAT2 gene
gcttgaacctcgaacaattgaag

SEQ ID NO: 215 - DNA, 25 bp, Artificial
  probe 3 for detecting the R197Q polymorphism in the NAT2 gene
cgcttgaacctcaaacaattgaaga

SEQ ID NO: 216 - DNA, 23 bp, Artificial
  probe 4 for detecting the R197Q polymorphism in the NAT2 gene
gcttgaacctcaaacaattgaag

SEQ ID NO: 217 - DNA, 25 bp, Artificial
  probe 1 for detecting the K268R polymorphism in the NAT2 gene
aagaagtgctgaaaaatatatttaa

SEQ ID NO: 218 - DNA, 25 bp, Artificial
  probe 2 for detecting the K268R polymorphism in the NAT2 gene
ttaaatatatttttcagcacttctt

SEQ ID NO: 219 - DNA, 25 bp, Artificial
  probe 3 for detecting the K268R polymorphism in the NAT2 gene
aagaagtgctgagaaatatatttaa

SEQ ID NO: 220 - DNA, 25 bp, Artificial
  probe 4 for detecting the K268R polymorphism in the NAT2 gene
ttaaatatatttctcagcacttctt

SEQ ID NO: 221 - DNA, 25 bp, Artificial
  probe 1 for detecting the G286E polymorphism in the NAT2 gene
aacctggtgatggatcccttactat

SEQ ID NO: 222 - DNA, 23 bp, Artificial
  probe 2 for detecting the G286E polymorphism in the NAT2 gene
acctggtgatggatcccttacta

SEQ ID NO: 223 - DNA, 25 bp, Artificial
  probe 3 for detecting the G286E polymorphism in the NAT2 gene
aacctggtgatgaatcccttactat

SEQ ID NO: 224 - DNA, 23 bp, Artificial
  probe 4 for detecting the G286E polymorphism in the NAT2 gene
acctggtgatgaatcccttacta

SEQ ID NO: 225 - DNA, 23 bp, Artificial
  probe 1 for detecting the -786 T>C polymorphism in the NOS3 gene
tcttccctggctggctgaccctg

SEQ ID NO: 226 - DNA, 23 bp, Artificial
  probe 2 for detecting the -786 T>C polymorphism in the NOS3 gene
cagggtcagccagccagggaaga

SEQ ID NO: 227 - DNA, 23 bp, Artificial
  probe 3 for detecting the -786 T>C polymorphism in the NOS3 gene
tcttccctggccggctgaccctg

SEQ ID NO: 228 - DNA, 23 bp, Artificial
  probe 4 for detecting the -786 T>C polymorphism in the NOS3 gene
cagggtcagccggccagggaaga

SEQ ID NO: 229 - DNA, 23 bp, Artificial
  probe 1 for detecting the Glu298Asp polymorphism in the NOS3 gene
gccccagatgagcccccagaact

SEQ ID NO: 230 - DNA, 23 bp, Artificial
  probe 2 for detecting the Glu298Asp polymorphism in the NOS3 gene
agttctgggggctcatctggggc

SEQ ID NO: 231 - DNA, 23 bp, Artificial
  probe 3 for detecting the Glu298Asp polymorphism in the NOS3 gene
gccccagatgatcccccagaact

SEQ ID NO: 232 - DNA, 23 bp, Artificial
  probe 4 for detecting the Glu298Asp polymorphism in the NOS3 gene
agttctgggggatcatctggggc

SEQ ID NO: 233 - DNA, 23 bp, Artificial
  probe 1 for detecting the Leu7Pro polymorphism in the NPY gene
cggacagccccagtcgcttgtta

SEQ ID NO: 234 - DNA, 23 bp, Artificial
  probe 2 for detecting the Leu7Pro polymorphism in the NPY gene
taacaagcgactggggctgtccg

SEQ ID NO: 235 - DNA, 23 bp, Artificial
  probe 3 for detecting the Leu7Pro polymorphism in the NPY gene
cggacagccccggtcgcttgtta

SEQ ID NO: 236 - DNA, 23 bp, Artificial
  probe 4 for detecting the Leu7Pro polymorphism in the NPY gene
taacaagcgaccggggctgtccg

SEQ ID NO: 237 - DNA, 23 bp, Artificial
  probe 1 for detecting the Cys326Ser polymorphism in the OGG1 gene
cctgcgccaatcccgccatgctc

SEQ ID NO: 238 - DNA, 21 bp, Artificial
  probe 2 for detecting the Cys326Ser polymorphism in the OGG1 gene
ctgcgccaatcccgccatgct

SEQ ID NO: 239 - DNA, 23 bp, Artificial
  probe 3 for detecting the Cys326Ser polymorphism in the OGG1 gene
cctgcgccaatgccgccatgctc

SEQ ID NO: 240 - DNA, 21 bp, Artificial
  probe 4 for detecting the Cys326Ser polymorphism in the OGG1 gene
ctgcgccaatgccgccatgct

SEQ ID NO: 241 - DNA, 17 bp, Artificial
  probe 1 for detecting the 4G>5G polymorphism in the PAI1 gene
ctgactcccccacgtgt

SEQ ID NO: 242 - DNA, 17 bp, Artificial
  probe 2 for detecting the 4G>5G polymorphism in the PAI1 gene
ggctgactcccccacgt

SEQ ID NO: 243 - DNA, 17 bp, Artificial
  probe 3 for detecting the 4G>5G polymorphism in the PAI1 gene
ctgactcccccacgtgt

SEQ ID NO: 244 - DNA, 17 bp, Artificial
  probe 4 for detecting the 4G>5G polymorphism in the PAI1 gene
cggctgactccccacgt

SEQ ID NO: 245 - DNA, 23 bp, Artificial
  probe 1 for detecting the 331 G>A polymorphism in the PGR gene
cgggagataaaagagccgcgtgt

SEQ ID NO: 246 - DNA, 23 bp, Artificial
  probe 2 for detecting the 331 G>A polymorphism in the PGR gene
acacgcggctcttttatctcccg

SEQ ID NO: 247 - DNA, 23 bp, Artificial
  probe 3 for detecting the 331 G>A polymorphism in the PGR gene
cgggagataaaggagccgcgtgt

SEQ ID NO: 248 - DNA, 23 bp, Artificial
  probe 4 for detecting the 331 G>A polymorphism in the PGR gene
acacgcggctcctttatctcccg

SEQ ID NO: 249 - DNA, 23 bp, Artificial
  probe 1 for detecting the Gln192Arg polymorphism in the PON1 gene
cccctacttacaatcctgggaga

SEQ ID NO: 250 - DNA, 23 bp, Artificial
  probe 2 for detecting the Gln192Arg polymorphism in the PON1 gene
tctcccaggattgtaagtagggg

SEQ ID NO: 251 - DNA, 23 bp, Artificial
  probe 3 for detecting the Gln192Arg polymorphism in the PON1 gene
cccctacttacgatcctgggaga

SEQ ID NO: 252 - DNA, 23 bp, Artificial
  probe 4 for detecting the Gln192Arg polymorphism in the PON1 gene
tctcccaggatcgtaagtagggg

SEQ ID NO: 253 - DNA, 23 bp, Artificial
  probe 1 for detecting the Ala16Val polymorphism in the SOD2 gene
gataccccaaagccggagccagc

SEQ ID NO: 254 - DNA, 21 bp, Artificial
  probe 2 for detecting the Ala16Val polymorphism in the SOD2 gene
ataccccaaagccggagccag

SEQ ID NO: 255 - DNA, 23 bp, Artificial
  probe 3 for detecting the Ala16Val polymorphism in the SOD2 gene
gataccccaaaaccggagccagc

SEQ ID NO: 256 - DNA, 21 bp, Artificial
  probe 4 for detecting the Ala16Val polymorphism in the SOD2 gene
ataccccaaaaccggagccag

SEQ ID NO: 257 - DNA, 23 bp, Artificial
  probe 1 for detecting the Ala49Thr polymorphism in the SRD5A2 gene
cccgcctgccagcccgcgccgcc

SEQ ID NO: 258 - DNA, 21 bp, Artificial
  probe 2 for detecting the Ala49Thr polymorphism in the SRD5A2 gene
ccgcctgccagcccgcgccgc

SEQ ID NO: 259 - DNA, 23 bp, Artificial
  probe 3 for detecting the Ala49Thr polymorphism in the SRD5A2 gene
cccgcctgccaacccgcgccgcc

SEQ ID NO: 260 - DNA, 21 bp, Artificial
  probe 4 for detecting the Ala49Thr polymorphism in the SRD5A2 gene
ccgcctgccaacccgcgccgc

SEQ ID NO: 261 - DNA, 21 bp, Artificial
  probe 1 for detecting the Val89Leu polymorphism in the SRD5A2 gene
cctcttctgcgtacattactt

SEQ ID NO: 262 - DNA, 19 bp, Artificial
  probe 2 for detecting the Val89Leu polymorphism in the SRD5A2 gene
ctcttctgcgtacattact

SEQ ID NO: 263 - DNA, 21 bp, Artificial
  probe 3 for detecting the Val89Leu polymorphism in the SRD5A2 gene
cctcttctgcctacattactt

SEQ ID NO: 264 - DNA, 19 bp, Artificial
  probe 4 for detecting the Val89Leu polymorphism in the SRD5A2 gene
ctcttctgcctacattact

SEQ ID NO: 265 - DNA, 21 bp, Artificial
  probe 1 for detecting the Gly595Ala polymorphism in the SREBF2 gene
gctgctgccggcaacctacaa

SEQ ID NO: 266 - DNA, 21 bp, Artificial
  probe 2 for detecting the Gly595Ala polymorphism in the SREBF2 gene
ttgtaggttgccggcagcagc

SEQ ID NO: 267 - DNA, 21 bp, Artificial
  probe 3 for detecting the Gly595Ala polymorphism in the SREBF2 gene
gctgctgccgccaacctacaa

SEQ ID NO: 268 - DNA, 21 bp, Artificial
  probe 4 for detecting the Gly595Ala polymorphism in the SREBF2 gene
ttgtaggttggcggcagcagc

SEQ ID NO: 269 - DNA, 21 bp, Artificial
  probe 1 for detecting the Arg213His polymorphism in the SULT1A1 gene
tttgtggggcgctccctgcca

SEQ ID NO: 270 - DNA, 19 bp, Artificial
  probe 2 for detecting the Arg213His polymorphism in the SULT1A1 gene
ttgtggggcgctccctgcc

SEQ ID NO: 271 - DNA, 21 bp, Artificial
  probe 3 for detecting the Arg213His polymorphism in the SULT1A1 gene
tttgtggggcactccctgcca

SEQ ID NO: 272 - DNA, 19 bp, Artificial
  probe 4 for detecting the Arg213His polymorphism in the SULT1A1 gene
ttgtggggcactccctgcc

SEQ ID NO: 273 - DNA, 23 bp, Artificial
  probe 1 for detecting the b>B polymorphism in the VDR gene
gacaggcctgcgcattcccaata

SEQ ID NO: 274 - DNA, 23 bp, Artificial
  probe 2 for detecting the b>B polymorphism in the VDR gene
tattgggaatgcgcaggcctgtc

SEQ ID NO: 275 - DNA, 23 bp, Artificial
  probe 3 for detecting the b>B polymorphism in the VDR gene
gacaggcctgcacattcccaata

SEQ ID NO: 276 - DNA, 23 bp, Artificial
  probe 4 for detecting the b>B polymorphism in the VDR gene
tattgggaatgtgcaggcctgtc

SEQ ID NO: 277 - DNA, 20 bp, Artificial
  oligonucleotide 1 for amplifying the fragment in which the Intron 16 ins/del polymorphism in the ACE gene may exist
gggactctgtaagccactgc

SEQ ID NO: 278 - DNA, 20 bp, Artificial
  oligonucleotide 2 for amplifying the fragment in which the Intron 16 ins/del polymorphism in the ACE gene may exist
ccatgcccataacaggtctt

SEQ ID NO: 279 - DNA, 21 bp, Artificial
  oligonucleotide 1 for amplifying the fragment in which the Gly389Arg polymorphism in the ADRB1 gene may exist
cgccttcaaccccatcatcta

SEQ ID NO: 280 - DNA, 18 bp, Artificial
  oligonucleotide 2 for amplifying the fragment in which the Gly389Arg polymorphism in the gene ADRB1 may exist
caggctcgagtcgctgtc

SEQ ID NO: 281 - DNA, 18 bp, Artificial
  oligonucleotide 1 for amplifying the fragment in which the Gln27Glu polymorphism in the ADRB2 gene may exist
gctcacctgccagactgc

SEQ ID NO: 282 - DNA, 20 bp, Artificial
  oligonucleotide 2 for amplifying the fragment in which the Gln27Glu polymorphism in the ADRB2 gene may exist
gccaggacgatgagagacat

SEQ ID NO: 283 - DNA, 18 bp, Artificial
  oligonucleotide 1 for amplifying the fragment in which the Gly16Arg polymorphism in the ADRB2 gene may exist
gctcacctgccagactgc

SEQ ID NO: 284 - DNA, 20 bp, Artificial
  oligonucleotide 2 for amplifying the fragment in which the Gly16Arg polymorphism in the ADRB2 gene may exist
gccaggacgatgagagacat

SEQ ID NO: 285 - DNA, 19 bp, Artificial
  oligonucleotide 1 for amplifying the fragment in which the Trp64Arg polymorphism in the ADRB3 gene may exist
caataccgccaacaccagt

SEQ ID NO: 286 - DNA, 19 bp, Artificial
  oligonucleotide 2 for amplifying the fragment in which the Trp64Arg polymorphism in the ADRB3 gene may exist
cgaagtcacgaacacgttg

SEQ ID NO: 287 - DNA, 20 bp, Artificial
  oligonucleotide 1 for amplifying the fragment in which the Met235Thr polymorphism in the AGT gene may exist
gaactggatgttgctgctga

SEQ ID NO: 288 - DNA, 20 bp, Artificial
  oligonucleotide 2 for amplifying the fragment in which the Met235Thr polymorphism in the AGT gene may exist
ttgccttaccttggaagtgg

SEQ ID NO: 289 - DNA, 20 bp, Artificial
  oligonucleotide 1 for amplifying the fragment in which the 1166 A>C polymorphism in the AGTR1 gene may exist
ccgcccctcagataatgtaa

SEQ ID NO: 290 - DNA, 20 bp, Artificial
  oligonucleotide 2 for amplifying the fragment in which the 1166 A>C polymorphism in the AGTR1 gene may exist
gcaaaatgtggctttgcttt

SEQ ID NO: 291 - DNA, 20 bp, Artificial
  oligonucleotide 1 for amplifying the fragment in which the -75 G>A polymorphism in the APOA1 gene may exist
cacctccttctcgcagtctc

SEQ ID NO: 292 - DNA, 20 bp, Artificial
  oligonucleotide 2 for amplifying the fragment in which the -75 G>A polymorphism in the APOA1 gene may exist
gggacagagctgatccttga

SEQ ID NO: 293 - DNA, 29 bp, Artificial
  oligonucleotide 1 for amplifying the fragment in which the Arg3480Trp polymorphism in the APOB gene may exist
agcctcacctcttacttttccattgagtc

SEQ ID NO: 294 - DNA, 24 bp, Artificial
  oligonucleotide 2 for amplifying the fragment in which the Arg3480Trp polymorphism in the APOB gene may exist
cgttggtgaaaaagaggccctcta

SEQ ID NO: 295 - DNA, 29 bp, Artificial
  oligonucleotide 1 for amplifying the fragment in which the Arg3500Gln polymorphism in the APOB gene may exist
agcctcacctcttacttttccattgagtc

SEQ ID NO: 296 - DNA, 24 bp, Artificial
  oligonucleotide 2 for amplifying the fragment in which the Arg3500Gln polymorphism in the APOB gene may exist
cgttggtgaaaaagaggccctcta

SEQ ID NO: 297 - DNA, 29 bp, Artificial
  oligonucleotide 1 for amplifying the fragment in which the Arg3531Cys polymorphism in the APOB gene may exist
agcctcacctcttacttttccattgagtc

SEQ ID NO: 298 - DNA, 24 bp, Artificial
  oligonucleotide 2 for amplifying the fragment in which the Arg3531Cys polymorphism in the APOB gene may exist
cgttggtgaaaaagaggccctcta

SEQ ID NO: 299 - DNA, 18 bp, Artificial
  oligonucleotide 1 for amplifying the fragment in which the Cys112Arg polymorphism in the APOE gene may exist
ctgtccaaggagctgcag

SEQ ID NO: 300 - DNA, 18 bp, Artificial
  oligonucleotide 2 for amplifying the fragment in which the Cys112Arg polymorphism in the APOE gene may exist
ctgttccaccaggggccc

SEQ ID NO: 301 - DNA, 18 bp, Artificial
  oligonucleotide 1 for amplifying the fragment in which the Arg158Cys polymorphism in the APOE gene may exist
ctgtccaaggagctgcag

SEQ ID NO: 302 - DNA, 18 bp, Artificial
  oligonucleotide 2 for amplifying the fragment in which the Arg158Cys polymorphism in the APOE gene may exist
ctgttccaccaggggccc

SEQ ID NO: 303 - DNA, 18 bp, Artificial
  oligonucleotide 1 for amplifying the fragment in which the 833 T>C polymorphism in the CBS gene may exist
gcttttgctggccttgag

SEQ ID NO: 304 - DNA, 20 bp, Artificial
  oligonucleotide 2 for amplifying the fragment in which the 833 T>C polymorphism in the CBS gene may exist
gggtgagttacaggctgcac

SEQ ID NO: 305 - DNA, 18 bp, Artificial
  oligonucleotide 1 for amplifying the fragment in which the 844ins68 polymorphism in the CBS gene may exist
gcttttgctggccttgag

SEQ ID NO: 306 - DNA, 20 bp, Artificial
  oligonucleotide 2 for amplifying the fragment in which the 844ins68 polymorphism in the CBS gene may exist
gggtgagttacaggctgcac

SEQ ID NO: 307 - DNA, 20 bp, Artificial
  oligonucleotide 1 for amplifying the fragment in which the TaqIB polymorphism in the CETP gene may exist
gcaaacagccaggtataggg

SEQ ID NO: 308 - DNA, 20 bp, Artificial
  oligonucleotide 2 for amplifying the fragment in which the TaqIB polymorphism in the CETP gene may exist
aagagactgaggcccagaga

SEQ ID NO: 309 - DNA, 20 bp, Artificial
  oligonucleotide 1 for amplifying the fragment in which the Arg451Gln polymorphism in the CETP gene may exist
gcaaacagccaggtataggg

SEQ ID NO: 310 - DNA, 20 bp, Artificial
  oligonucleotide 2 for amplifying the fragment in which the Arg451Gln polymorphism in the CETP gene may exist
aagagactgaggcccagaga

SEQ ID NO: 311 - DNA, 20 bp, Artificial
  oligonucleotide 1 for amplifying the fragment in which the 1546 G>T polymorphism in the COL1A1 gene may exist
agccgctcccattctcttag

SEQ ID NO: 312 - DNA, 20 bp, Artificial
  oligonucleotide 2 for amplifying the fragment in which the 1546 G>T polymorphism in the COL1A1 gene may exist
gcgtggtagagacaggagga

SEQ ID NO: 313 - DNA, 20 bp, Artificial
  oligonucleotide 1 for amplifying the fragment in which the Val158Met (Allele*2) polymorphism in the COMT gene may exist
gggcctactgtggctactca

SEQ ID NO: 314 - DNA, 20 bp, Artificial
  oligonucleotide 2 for amplifying the fragment in which the Val158Met (Allele*2) polymorphism in the COMT gene may exist
ccctttttccaggtctgaca

SEQ ID NO: 315 - DNA, 20 bp, Artificial
  oligonucleotide 1 for amplifying the fragment in which the -34 A>G polymorphism in the CYP17A1 gene may exist
gggctccaggagaatctttc

SEQ ID NO: 316 - DNA, 20 bp, Artificial
  oligonucleotide 2 for amplifying the fragment in which the -34 A>G polymorphism in the CYP17A1 gene may exist
agggtaagcagcaagagagc

SEQ ID NO: 317 - DNA, 20 bp, Artificial
  oligonucleotide 1 for amplifying the fragment in which the 1558 C>T polymorphism in the CYP19A1 gene may exist
ccttgcacccagatgagact

SEQ ID NO: 318 - DNA, 20 bp, Artificial
  oligonucleotide 2 for amplifying the fragment in which the 1558 C>T polymorphism in the CYP19A1 gene may exist
ggcaaggatggatgatttgt

SEQ ID NO: 319 - DNA, 20 bp, Artificial
  oligonucleotide 1 for amplifying the fragment in which the Ile462Val polymorphism in the CYP1A1 gene may exist
tgatggtgctatcgacaagg

SEQ ID NO: 320 - DNA, 20 bp, Artificial
  oligonucleotide 2 for amplifying the fragment in which the Ile462Val polymorphism in the CYP1A1 gene may exist
tttggaagtgctcacagcag

SEQ ID NO: 321 - DNA, 20 bp, Artificial
  oligonucleotide 1 for amplifying the fragment in which the T3801C polymorphism in the CYP1A1 gene may exist
ccgctgcacttaagcagtct

SEQ ID NO: 322 - DNA, 19 bp, Artificial
  oligonucleotide 2 for amplifying the fragment in which the T3801C polymorphism in the CYP1A1 gene may exist
ggccccaactactcagagg

SEQ ID NO: 323 - DNA, 20 bp, Artificial
  oligonucleotide 1 for amplifying the fragment in which the Leu432Val polymorphism in the CYP1B1 gene may exist
acctctgtcttgggctacca

SEQ ID NO: 324 - DNA, 20 bp, Artificial
  oligonucleotide 2 for amplifying the fragment in which the Leu432Val polymorphism in the CYP1B1 gene may exist
gccaggatggagatgaagag

SEQ ID NO: 325 - DNA, 20 bp, Artificial
  oligonucleotide 1 for amplifying the fragment in which the Allele*4 (Asn453Ser) polymorphism in the CYP1B1 gene may exist
acctctgtcttgggctacca

SEQ ID NO: 326 - DNA, 20 bp, Artificial
  oligonucleotide 2 for amplifying the fragment in which the Allele*4 (Asn453Ser) polymorphism in the CYP1B1 gene may exist
gccaggatggagatgaagag

SEQ ID NO: 327 - DNA, 20 bp, Artificial
  oligonucleotide 1 for amplifying the fragment in which the Arg144Cys (allele*2) polymorphism in the CYP2C9 gene may exist
cctgggatctccctcctagt

SEQ ID NO: 328 - DNA, 20 bp, Artificial
  oligonucleotide 2 for amplifying the fragment in which the Arg144Cys (allele*2) polymorphism in the CYP2C9 gene may exist
ccacccttggtttttctcaa

SEQ ID NO: 329 - DNA, 20 bp, Artificial
  oligonucleotide 1 for amplifying the fragment in which the Ile359Leu (allele*3) polymorphism in the CYP2C9 gene may exist
ccacatgccctacacagatg

SEQ ID NO: 330 - DNA, 20 bp, Artificial
  oligonucleotide 2 for amplifying the fragment in which the p Ile359Leu (allele*3) olymorphism in the CYP2C9 gene may exist
tcgaaaacatggagttgcag

SEQ ID NO: 331 - DNA, 21 bp, Artificial
  oligonucleotide 1 for amplifying the fragment in which the 681 G>A (Pro227Pro) (allele*2) polymorphism in the CYP2C19 gene may exist
caaccagagcttggcatattg

SEQ ID NO: 332 - DNA, 20 bp, Artificial
  oligonucleotide 2 for amplifying the fragment in which the 681 G>A (Pro227Pro) (allele*2) polymorphism in the CYP2C19 gene may exist
taaagtcccgagggttgttg

SEQ ID NO: 333 - DNA, 20 bp, Artificial
  oligonucleotide 1 for amplifying the fragment in which the 2549 A>del (allele*3) polymorphism in the CYP2D6 gene may exist
gggcctgagacttgtccagg

SEQ ID NO: 334 - DNA, 20 bp, Artificial
  oligonucleotide 2 for amplifying the fragment in which the 2549 A>del (allele*3) polymorphism in the CYP2D6 gene may exist
gccgagagcatactcgggac

SEQ ID NO: 335 - DNA, 22 bp, Artificial
  oligonucleotide 1 for amplifying the fragment in which the 1846 G>A / 1847 G>A (allele*4) polymorphism in the CYP2D6 gene may exist
ccacgcgcacgtgcccgtccca

SEQ ID NO: 336 - DNA, 24 bp, Artificial
  oligonucleotide 2 for amplifying the fragment in which the 1846 G>A / 1847 G>A (allele*4) polymorphism in the CYP2D6 gene may exist
cctgcagagactcctcggtctctc

SEQ ID NO: 337 - DNA, 22 bp, Artificial
  oligonucleotide 1 for amplifying the fragment in which the 1707 del>T (allele*6) polymorphism in the CYP2D6 gene may exist
ccacgcgcacgtgcccgtccca

SEQ ID NO: 338 - DNA, 24 bp, Artificial
  oligonucleotide 2 for amplifying the fragment in which the 1707 del>T (allele*6) polymorphism in the CYP2D6 gene may exist
cctgcagagactcctcggtctctc

SEQ ID NO: 339 - DNA, 20 bp, Artificial
  oligonucleotide 1 for amplifying the fragment in which the Ala541Thr polymorphism in the ELAC2 gene may exist
ccgacacgtctctgctactg

SEQ ID NO: 340 - DNA, 20 bp, Artificial
  oligonucleotide 2 for amplifying the fragment in which the Ala541Thr polymorphism in the ELAC2 gene may exist
aacaaaagctctgggcaagt

SEQ ID NO: 341 - DNA, 20 bp, Artificial
  oligonucleotide 1 for amplifying the fragment in which the -397 T>C (PvuII) polymorphism in the ESR1 IVS1 gene may exist
agggttatgtggcaatgacg

SEQ ID NO: 342 - DNA, 20 bp, Artificial
  oligonucleotide 2 for amplifying the fragment in which the -397 T>C (PvuII) polymorphism in the ESR1 IVS1 gene may exist
accaatgctcatcccaactc

SEQ ID NO: 343 - DNA, 23 bp, Artificial
  oligonucleotide 1 for amplifying the fragment in which the Val34Leu polymorphism in the F13A1 gene may exist
catgccttttctgttgtcttctt

SEQ ID NO: 344 - DNA, 20 bp, Artificial
  oligonucleotide 2 for amplifying the fragment in which the Val34Leu polymorphism in the F13A1 gene may exist
cccagtggagacagaggatg

SEQ ID NO: 345 - DNA, 25 bp, Artificial
  oligonucleotide 1 for amplifying the fragment in which the -455 G>A polymorphism in the FGB gene may exist
gggtctttctgatgtgtatttttca

SEQ ID NO: 346 - DNA, 21 bp, Artificial
  oligonucleotide 2 for amplifying the fragment in which the -455 G>A polymorphism in the FGB gene may exist
gacctactcacaaggcaacca

SEQ ID NO: 347 - DNA, 20 bp, Artificial
  oligonucleotide 1 for amplifying the fragment in which the 20210 G>A polymorphism in the FII gene may exist
gagagtagggggccactcat

SEQ ID NO: 348 - DNA, 18 bp, Artificial
  oligonucleotide 2 for amplifying the fragment in which the 20210 G>A polymorphism in the FII gene may exist
cctgagcccagagagctg

SEQ ID NO: 349 - DNA, 20 bp, Artificial
  oligonucleotide 1 for amplifying the fragment in which the Arg506Gln polymorphism in the FV Leiden gene may exist
gcccagtgcttaacaagacc

SEQ ID NO: 350 - DNA, 22 bp, Artificial
  oligonucleotide 2 for amplifying the fragment in which the Arg506Gln polymorphism in the FV Leiden gene may exist
cccattatttagccaggagacc

SEQ ID NO: 351 - DNA, 20 bp, Artificial
  oligonucleotide 1 for amplifying the fragment in which the Pro319Ser polymorphism in the GJA4 gene may exist
cctcctcagacccttacacg

SEQ ID NO: 352 - DNA, 20 bp, Artificial
  oligonucleotide 2 for amplifying the fragment in which the Pro319Ser polymorphism in the GJA4 gene may exist
gcagccagacttctcaggac

SEQ ID NO: 353 - DNA, 20 bp, Artificial
  oligonucleotide 1 for amplifying the fragment in which the 393 T>C (Ile131Ile) polymorphism in the GNAS gene may exist
agtacgtgctggctccttgt

SEQ ID NO: 354 - DNA, 20 bp, Artificial
  oligonucleotide 2 for amplifying the fragment in which the 393 T>C (Ile131Ile) polymorphism in the GNAS gene may exist
cacaagtcggggtgtagctt

SEQ ID NO: 355 - DNA, 18 bp, Artificial
  oligonucleotide 1 for amplifying the fragment in which the 825 C>T (Ser275Ser) polymorphism in the GNB3 gene may exist
ctgccgcttgtttgacct

SEQ ID NO: 356 - DNA, 20 bp, Artificial
  oligonucleotide 2 for amplifying the fragment in which the 825 C>T (Ser275Ser) polymorphism in the GNB3 gene may exist
cacacgctcagacttcatgg

SEQ ID NO: 357 - DNA, 21 bp, Artificial
  oligonucleotide 1 for amplifying the fragment in which the GSTM1 polymorphism may exist
tgcttcacgtgttatggaggt

SEQ ID NO: 358 - DNA, 20 bp, Artificial
  oligonucleotide 2 for amplifying the fragment in which the GSTM1 polymorphism may exist
gggctcaaatatacggtgga

SEQ ID NO: 359 - DNA, 20 bp, Artificial
  oligonucleotide 1 for amplifying the fragment in which the Ile105Val polymorphism in the GSTP1 gene may exist
ctctatgggaaggaccagca

SEQ ID NO: 360 - DNA, 20 bp, Artificial
  oligonucleotide 2 for amplifying the fragment in which the Ile105Val polymorphism in the GSTP1 gene may exist
gaagcccctttctttgttca

SEQ ID NO: 361 - DNA, 20 bp, Artificial
  oligonucleotide 1 for amplifying the fragment in which the Ala114Val polymorphism in the GSTP1 gene may exist
gcaagcagaggagaatctgg

SEQ ID NO: 362 - DNA, 20 bp, Artificial
  oligonucleotide 2 for amplifying the fragment in which the Ala114Val polymorphism in the GSTP1 gene may exist
ctcacctggtctcccacaat

SEQ ID NO: 363 - DNA, 20 bp, Artificial
  oligonucleotide 1 for amplifying the fragment in which the GSTT1 polymorphism may exist
ggcagcataagcaggacttc

SEQ ID NO: 364 - DNA, 20 bp, Artificial
  oligonucleotide 2 for amplifying the fragment in which the GSTT1 polymorphism may exist
ctgcagttgctcgaggacaa

SEQ ID NO: 365 - DNA, 20 bp, Artificial
  oligonucleotide 1 for amplifying the fragment in which the -174 C>G polymorphism in the IL6 gene may exist
gcctcaatgacgacctaagc

SEQ ID NO: 366 - DNA, 20 bp, Artificial
  oligonucleotide 2 for amplifying the fragment in which the -174 C>G polymorphism in the IL6 gene may exist
tcatgggaaaatcccacatt

SEQ ID NO: 367 - DNA, 20 bp, Artificial
  oligonucleotide 1 for amplifying the fragment in which the -1082 G>A polymorphism in the IL10 gene may exist
tccccaggtagagcaacact

SEQ ID NO: 368 - DNA, 20 bp, Artificial
  oligonucleotide 2 for amplifying the fragment in which the -1082 G>A polymorphism in the IL10 gene may exist
atggaggctggataggaggt

SEQ ID NO: 369 - DNA, 20 bp, Artificial
  oligonucleotide 1 for amplifying the fragment in which the Leu33Pro polymorphism in the ITGB3 gene may exist
gctccaatgtacggggtaaa

SEQ ID NO: 370 - DNA, 20 bp, Artificial
  oligonucleotide 2 for amplifying the fragment in which the Leu33Pro polymorphism in the ITGB3 gene may exist
actcactgggaactcgatgg

SEQ ID NO: 371 - DNA, 20 bp, Artificial
  oligonucleotide 1 for amplifying the fragment in which the 5A>6A polymorphism in the MMP3 gene may exist
tcactgccaccactctgttc

SEQ ID NO: 372 - DNA, 20 bp, Artificial
  oligonucleotide 2 for amplifying the fragment in which the 5A>6A polymorphism in the MMP3 gene may exist
gcctcaacctctcaaagtgc

SEQ ID NO: 373 - DNA, 20 bp, Artificial
  oligonucleotide 1 for amplifying the fragment in which the Ala222Val polymorphism in the MTHFR gene may exist
gcctctcctgactgtcatcc

SEQ ID NO: 374 - DNA, 20 bp, Artificial
  oligonucleotide 2 for amplifying the fragment in which the Ala222Val polymorphism in the MTHFR gene may exist
caaagcggaagaatgtgtca

SEQ ID NO: 375 - DNA, 20 bp, Artificial
  oligonucleotide 1 for amplifying the fragment in which the R64Q polymorphism in the NAT2 gene may exist
ccatggagttgggcttagag

SEQ ID NO: 376 - DNA, 20 bp, Artificial
  oligonucleotide 2 for amplifying the fragment in which the R64Q polymorphism in the NAT2 gene may exist
ccatgccagtgctgtatttg

SEQ ID NO: 377 - DNA, 20 bp, Artificial
  oligonucleotide 1 for amplifying the fragment in which the 282 C>T (Y94Y) polymorphism in the NAT2 gene may exist
ccatggagttgggcttagag

SEQ ID NO: 378 - DNA, 20 bp, Artificial
  oligonucleotide 2 for amplifying the fragment in which the 282 C>T (Y94Y) polymorphism in the NAT2 gene may exist
ccatgccagtgctgtatttg

SEQ ID NO: 379 - DNA, 20 bp, Artificial
  oligonucleotide 1 for amplifying the fragment in which the I114T polymorphism in the NAT2 gene may exist
ccatggagttgggcttagag

SEQ ID NO: 380 - DNA, 20 bp, Artificial
  oligonucleotide 2 for amplifying the fragment in which the I114T polymorphism in the NAT2 gene may exist
ccatgccagtgctgtatttg

SEQ ID NO: 381 - DNA, 19 bp, Artificial
  oligonucleotide 1 for amplifying the fragment in which the 481C>T(L161L) polymorphism in the NAT2 gene may exist
caggtgccttgcattttct

SEQ ID NO: 382 - DNA, 20 bp, Artificial
  oligonucleotide 2 for amplifying the fragment in which the 481C>T (L161L) polymorphism in the NAT2 gene may exist
gatgaagcccaccaaacagt

SEQ ID NO: 383 - DNA, 19 bp, Artificial
  oligonucleotide 1 for amplifying the fragment in which the R197Q polymorphism in the NAT2 gene may exist
caggtgccttgcattttct

SEQ ID NO: 384 - DNA, 20 bp, Artificial
  oligonucleotide 2 for amplifying the fragment in which the R197Q polymorphism in the NAT2 gene may exist
gatgaagcccaccaaacagt

SEQ ID NO: 385 - DNA, 24 bp, Artificial
  oligonucleotide 1 for amplifying the fragment in which the K268R polymorphism in the NAT2 gene may exist
aaagacaatacagatctggtcgag

SEQ ID NO: 386 - DNA, 24 bp, Artificial
  oligonucleotide 2 for amplifying the fragment in which the K268R polymorphism in the NAT2 gene may exist
tcttcaaaataacgtgagggtaga

SEQ ID NO: 387 - DNA, 24 bp, Artificial
  oligonucleotide 1 for amplifying the fragment in which the G286E polymorphism in the NAT2 gene may exist
aaagacaatacagatctggtcgag

SEQ ID NO: 388 - DNA, 24 bp, Artificial
  oligonucleotide 2 for amplifying the fragment in which the G286E polymorphism in the NAT2 gene may exist
tcttcaaaataacgtgagggtaga

SEQ ID NO: 389 - DNA, 20 bp, Artificial
  oligonucleotide 1 for amplifying the fragment in which the -786 T>C polymorphism in the NOS3 gene may exist
gtgtaccccacctgcattct

SEQ ID NO: 390 - DNA, 19 bp, Artificial
  oligonucleotide 2 for amplifying the fragment in which the -786 T>C polymorphism in the NOS3 gene may exist
cccaccctgtcattcagtg

SEQ ID NO: 391 - DNA, 20 bp, Artificial
  oligonucleotide 1 for amplifying the fragment in which the Glu298Asp polymorphism in the NOS3 gene may exist
gaaggcaggagacagtggat

SEQ ID NO: 392 - DNA, 20 bp, Artificial
  oligonucleotide 2 for amplifying the fragment in which the Glu298Asp polymorphism in the NOS3 gene may exist
cagtcaatccctttggtgct

SEQ ID NO: 393 - DNA, 20 bp, Artificial
  oligonucleotide 1 for amplifying the fragment in which the Leu7Pro polymorphism in the NPY gene may exist
ctctgcctggtgatgaggtt

SEQ ID NO: 394 - DNA, 19 bp, Artificial
  oligonucleotide 2 for amplifying the fragment in which the Leu7Pro polymorphism in the NPY gene may exist
ggtgctctgaatccccaag

SEQ ID NO: 395 - DNA, 20 bp, Artificial
  oligonucleotide 1 for amplifying the fragment in which the Cys326Ser polymorphism in the OGG1 gene may exist
tagtctcaccagccctgacc

SEQ ID NO: 396 - DNA, 20 bp, Artificial
  oligonucleotide 2 for amplifying the fragment in which the Cys326Ser polymorphism in the OGG1 gene may exist
tggggaatttctttgtccag

SEQ ID NO: 397 - DNA, 20 bp, Artificial
  oligonucleotide 1 for amplifying the fragment in which the 4G>5G polymorphism in the PAI1 gene may exist
caacctcagccagacaaggt

SEQ ID NO: 398 - DNA, 20 bp, Artificial
  oligonucleotide 2 for amplifying the fragment in which the 4G>5G polymorphism in the PAI1 gene may exist
cagccacgtgattgtctagg

SEQ ID NO: 399 - DNA, 20 bp, Artificial
  oligonucleotide 1 for amplifying the fragment in which the 331 G>A polymorphism in the PGR gene may exist
gcttcacagcatgcacgagt

SEQ ID NO: 400 - DNA, 20 bp, Artificial
  oligonucleotide 2 for amplifying the fragment in which the 331 G>A polymorphism in the PGR gene may exist
tattgttgctgtgggacctg

SEQ ID NO: 401 - DNA, 20 bp, Artificial
  oligonucleotide 1 for amplifying the fragment in which the Gln192Arg polymorphism in the PON1 gene may exist
tattgttgctgtgggacctg

SEQ ID NO: 402 - DNA, 20 bp, Artificial
  oligonucleotide 2 for amplifying the fragment in which the Gln192Arg polymorphism in the PON1 gene may exist
caaatccttctgccaccact

SEQ ID NO: 403 - DNA, 20 bp, Artificial
  oligonucleotide 1 for amplifying the fragment in which the Ala16Val polymorphism in the SOD2 gene may exist
ggctgtgctttctcgtcttc

SEQ ID NO: 404 - DNA, 19 bp, Artificial
  oligonucleotide 2 for amplifying the fragment in which the Ala16Val polymorphism in the SOD2 gene may exist
ccgtagtcgtagggcaggt

SEQ ID NO: 405 - DNA, 18 bp, Artificial
  oligonucleotide 1 for amplifying the fragment in which the Ala49Thr polymorphism in the SRD5A2 gene may exist
agcacacggagagcctga

SEQ ID NO: 406 - DNA, 20 bp, Artificial
  oligonucleotide 2 for amplifying the fragment in which the Ala49Thr polymorphism in the SRD5A2 gene may exist
aggggaaaaacgctacctgt

SEQ ID NO: 407 - DNA, 18 bp, Artificial
  oligonucleotide 1 for amplifying the fragment in which the Val89Leu polymorphism in the SRD5A2 gene may exist
agcacacggagagcctga

SEQ ID NO: 408 - DNA, 20 bp, Artificial
  oligonucleotide 2 for amplifying the fragment in which the Val89Leu polymorphism in the SRD5A2 gene may exist
aggggaaaaacgctacctgt

SEQ ID NO: 409 - DNA, 20 bp, Artificial
  oligonucleotide 1 for amplifying the fragment in which the Gly595Ala polymorphism in the SREBF2 gene may exist
ggccagtgaccattaacacc

SEQ ID NO: 410 - DNA, 20 bp, Artificial
  oligonucleotide 2 for amplifying the fragment in which the Gly595Ala polymorphism in the SREBF2 gene may exist
tcttcaaagcctgcctcagt

SEQ ID NO: 411 - DNA, 20 bp, Artificial
  oligonucleotide 1 for amplifying the fragment in which the Arg213His polymorphism in the SULT1A1 gene may exist
gtaatccgagcctccactga

SEQ ID NO: 412 - DNA, 20 bp, Artificial
  oligonucleotide 2 for amplifying the fragment in which the Arg213His polymorphism in the SULT1A1 gene may exist
gctgtggtccatgaactcct

SEQ ID NO: 413 - DNA, 20 bp, Artificial
  oligonucleotide 1 for amplifying the fragment in which the b>B polymorphism in the VDR gene may exist
cctcactgcccttagctctg

SEQ ID NO: 414 - DNA, 20 bp, Artificial
  oligonucleotide 2 for amplifying the fragment in which the b>B polymorphism in the VDR gene may exist
cccgcaagaaacctcaaata

SEQ ID NO: 415 - DNA, 50 bp, Artificial
  nucleotide sequence of the external control CEH
gtcgtcaagatgctaccgttcaggagtcgtcaagatgctaccgttcagga

SEQ ID NO: 416 - DNA, 19 bp, Artificial
  oligonucleotide 1 for detecting the external control
cttgacgactcctgaacgg

SEQ ID NO: 417 - DNA, 19 bp, Artificial
  oligonucleotido 2 for detecting the external control
cttgacgacacctgaacgg